(19) United States
(12) Patent Application Publication
(10) Pub. No.: US 2008/0051420 A1
(43) Pub. Date: Feb. 28, 2008

(54) New Compounds 317

(75) Inventors: Stefan Berg, Sodertalje (SE); Katharina Hogdin, Sodertalje (SE); Jacob Kihlstrom, Sodertalje (SE); Niklas Plobeck, Sodertalje (SE); Fernando Sehgelmeble, Sodertalje (SE); Maria Wirstam, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignees: AstraZeneca AB, Sodertalje (SE); Astex Therapeutics Ltd, Cambridge (GB)

(21) Appl. No.: 11/761,130

(22) Filed: Jun. 11, 2007

Related U.S. Application Data
(60) Provisional application No. 60/813,728, filed on Jun. 14, 2006.
(60) Provisional application No. 60/896,994, filed on Mar. 26, 2007.

(51) Int. Cl. A61K 31/519; A61K 31/415; A61P 25/00; C07D 237/04; C07D 487/04; C12N 9/50

(52) U.S. Cl. 514/259.1; 435/219; 514/393; 544/281; 548/303.1

(57) ABSTRACT

This invention relates to novel compounds having the structural formula I below: 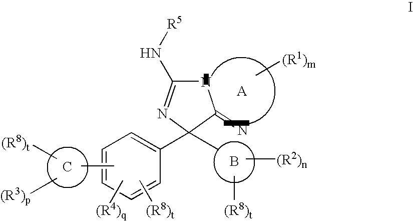 and to their pharmaceutically acceptable salt, compositions and methods of use. These novel compounds provide a treatment or prophylaxis of cognitive impairment, Alzheimer Disease, neurodegeneration and dementia.

The present invention relates to novel compounds, their pharmaceutical compositions. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”, Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with 7-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients. The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”, Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO05/097767, WO06/041404, WO06/041405, WO06/0065204, WO06/0065277, US2006287294, WO06/138265, US20050282826, US20050282825, US20060281729, WO06/138217, WO06/138230, WO06/138264, WO06/138265, WO06/138266, WO06/099379, WO06/076284, US20070004786, US20070004730, WO07/011,833, WO07/011,810, US20070099875, US20070099898, WO07/049,532).

The compounds of the present invention show beneficial properties compared to the potential inhibitors known in the art, e.g. improved hERG selectivity.

### DISCLOSURE OF THE INVENTION

Provided herein are novel compounds of structural formula J: 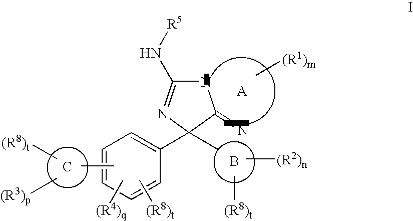
A is independently selected from a 5, 6 or 7 membered heterocyclic ring optionally substituted with one or more R¹;
B is independently selected from phenyl or from a 5 or 6 membered heteroaromatic ring optionally substituted with one or more R²;
C is independently selected from phenyl or a 5 or 6 membered heteroaromatic ring optionally substituted with one or more R³;
R¹ is independently selected from halogen, cyano, nitro, OR⁶, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl, NR⁶R⁷, CONR⁶R⁷, NR⁶(CO)R⁷, O(CO)R⁶, CO₂R⁶, COR⁶, (SO₂)NR⁶R⁷, NR⁶(SO₂)R⁷, SOR⁶, SO₂R⁶, OSO₂R⁶ and SO₃R⁶ wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl and C₀₋₆alkylC₃₋₆heterocyclyl is optionally substituted with one or more E; or two R¹ substituents together with the atom to which they are attached, form a cyclic or heterocyclic ring optionally substituted with one or more E;
R², R³ or R⁴ is selected from aryl, heteroaryl, C₃₋₆cycloalkenyl, C₃₋₆cycloalkynyl, C₃₋₆heterocyclyl, CONR⁶R⁷, NR⁶(CO)R⁷, O(CO)R⁶, CO₂R⁶, COR⁶, (SO₂)NR⁶R⁷, NR⁶(SO₂)R⁷, SOR⁶, SO₂R⁶, OSO₂R⁶ and SO₃R⁶ wherein said aryl, heteroaryl, C₃₋₆cycloalkenyl, C₃₋₆cycloalkynyl and C₃₋₆heterocyclyl may be optionally substituted with one or more E;
R⁵ is independently selected from hydrogen, cyano, OR⁶, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl, CONR⁶R⁷, CO₂R⁶, COR⁶, SO₂R⁶ and SO₃R⁶ wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl may be optionally substituted with one or more E;
E is independently selected from halogen, nitro, CN, OR⁶, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl, fluoromethyl, difluoromethyl, trifluoromethyl, fluoromethoxy, difluoromethoxy, trifluoromethoxy, NR⁶R₇, CONR⁶R⁷, NR⁶(CO)R⁷, O(CO)R⁶, CO₂R⁶, COR⁶, (SO₂)NR⁶R⁷, NR⁶SO₂R⁷, SO₂R⁶, SOR⁶, OSO₂R⁶ and SO₃R⁶, wherein said C₁₋₆alkyl, C₂₋₆alkenyl, C₂₋₆alkynyl, C₀₋₆-alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl or C₀₋₆alkylheterocyclyl may be optionally substituted with one or more substituents independently selected from halo, nitro, cyano, OR⁶, C₁₋₆alkyl, fluoromethyl, difluoromethyl, trifluoromethyl, fluoromethoxy, difluoromethoxy and trifluoromethoxy;
R⁶ and R⁷ are independently selected from hydrogen, C₁₋₆alkyl, fluoromethyl, difluoromethyl, trifluoromethyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl, or
R⁶ and R⁷ may together form a 5 or 6 membered heterocyclic ring containing one or more heteroatoms selected from N, O or S;
R⁸ is independently selected from halogen, cyano, nitro, OR⁹, C₁₋₆alkyl, C₂₋₆alkenyl, is C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl, NR⁹R¹⁰, CONR⁹R¹⁰, NR⁹(CO)R¹⁰, O(CO)R⁹, CO₂R⁹, COR⁹, (SO₂)NR⁹R¹⁰, NR⁹(SO₂)R¹⁰, SO₂R⁹, SOR⁹, OSO₂R⁹ and SO₃R⁹ wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl and C₀₋₆alkylC₃₋₆heterocyclyl may be optionally substituted with one or more E; or
two R⁸ may together with the atoms to which they are attached form a cyclic or heterocyclic ring optionally substituted with one or more E;
R⁹ and R¹⁰ are independently selected from hydrogen, C₁₋₆alkyl, fluoromethyl, difluoromethyl, trifluoromethyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl; or
R⁹ and R¹⁰ may together form a 5 or 6 membered heterocyclic ring containing one or more heteroatoms selected from N, O or S;
m=0, 1 or 2;
n=0, 1, 2 or 3;
p=0, 1, 2 or 3;
q=0, 1, 2 or 3;
t=0, 1, 2 or 3;
wherein one of n, p or q is at least 1;
as a free base or a pharmaceutically acceptable salt, solvate or solvate of a salt thereof.

The present invention further provides pharmaceutical compositions comprising as active ingredient a therapeutically effective amount of a compound of formula I in association with pharmaceutically acceptable excipients, carriers or diluents.

The present invention further provides methods of modulating activity of BACE comprising contacting the BACE enzyme with a compound of formula I.

The present invention further provides methods of treating or preventing an Aβ-related pathology in a patient, comprising administering to the patient a therapeutically effective amount of a compound of formula I.

The present invention further provides a compound described herein for use as a medicament.

In one aspect of the present invention, there is provided a compound according to formula I, wherein

A is independently selected from a 5 or 6 membered heterocyclic ring;

B is independently selected from phenyl or from a 5 or 6 membered heteroaromatic ring optionally substituted with one or more R²;

C is independently selected from phenyl or a 5 or 6 membered heteroaromatic ring optionally substituted with one or more R³;

R², R³ or R⁴ is independently selected from aryl, heteroaryl, C₃₋₆cycloalkenyl, C₃₋₆cycloalkynyl, C₃₋₆heterocyclyl, CONR⁶R⁷, NR⁶(CO)R⁷, O(CO)R⁶, CO₂R⁶, COR⁶, (SO₂)NR⁶R⁷, NR⁶(SO₂)R⁷, SOR⁶, SO₂R⁶, OSO₂R⁶ and SO₃R⁶ wherein said aryl, heteroaryl, C₃₋₆cycloalkenyl, C₃₋₆cycloalkynyl and C₃₋₆heterocyclyl may be optionally substituted with one or more E;

R⁸ is hydrogen;

E is independently selected from halogen, nitro, CN, OR⁶, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl, fluoromethyl, difluoromethyl, trifluoromethyl, fluoromethoxy, difluoromethoxy, trifluoromethoxy, NR⁶R⁷. CONR⁶R⁷ NR⁶(CO)R⁷, O(CO)R⁶, CO₂R⁶, COR⁶, (SO₂)NR⁶R⁷, NR⁶SO₂R⁷, SO₂R⁶, SOR⁶, OSO₂R⁶ and SO₃R⁶, wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl or C₀₋₆alkylheterocyclyl may be optionally substituted with one or more substituents independently selected from halo, nitro, cyano, OR⁶, C₁₋₆alkyl, fluoromethyl, difluoromethyl, trifluoromethyl, fluoromethoxy, difluoromethoxy and trifluoromethoxy;

R⁶ and R⁷ are independently selected from hydrogen, C₁₋₆alkyl, fluoromethyl, difluoromethyl, trifluoromethyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl, or

R⁶ and R⁷ may together form a 5 or 6 membered heterocyclic ring containing one or more heteroatoms selected from N, O or S;

R³ is independently selected from halogen, cyano, nitro, OR⁹, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl, NR⁹R¹⁰, CONR⁹R¹⁰, NR⁹(CO)R¹⁰, O(CO)R⁹, CO₂R⁹, COR⁹, (SO₂)NR⁹R¹⁰, NR⁹(SO₂)R¹⁰, SO₂R⁹, SOR⁹, OSO₂R⁹ and SO₃R⁹ wherein said C₁₋₆alkyl, C₂₋₆alkenyl, C₂₋₆alkynyl, C₀₋₆alkylaryl, C₀₋₆-alkylheteroaryl, C₀₋₆alkylC₃₋₆cycloalkyl, C₀₋₆alkylC₃₋₆cycloalkenyl, C₀₋₆alkylC₃₋₆cycloalkynyl and C₀₋₆alkylC₃₋₆heterocyclyl may be optionally substituted with one or more E; or

two R⁸ may together with the atoms to which they are attached form a cyclic or heterocyclic ring optionally substituted with one or more E;

R⁹ and R¹⁰ are independently selected from hydrogen, C₁₋₆alkyl, fluoromethyl, difluoromethyl, trifluoromethyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl; or

R⁹ and R¹⁰ may together form a 5 or 6 membered heterocyclic ring containing one or more heteroatoms selected from N, O or S;

m=0

n=0 or 1;

p=0 or 1;

q=0, 1, 2 or 3;

t=0, 1, 2 or 3;

wherein one of n, p or q is at least 1.

In another aspect of the present invention, there is provided a compound according to formula I, wherein B is independently selected from phenyl or a 6 membered heteroaromatic ring optionally substituted with one R².

In another aspect of the present invention, there is provided a compound according to formula I, wherein B is independently selected from phenyl and pyridyl optionally substituted with one R².

In another aspect of the present invention, there is provided a compound according to formula I, wherein C is independently selected from phenyl or a 6 membered heteroaromatic ring optionally substituted with one R³.

In another aspect of the present invention, there is provided a compound according to formula I, wherein n is 1 and R² is OSO₂R⁶.

In another aspect of the present invention, there is provided a compound according to formula I, wherein B is independently selected from phenyl and pyridyl; n is 1 and R² is OSO₂R⁶

In another aspect of the present invention, there is provided a compound according to formula I, wherein R³ is OSO₂R⁶.

In another aspect of the present invention, there is provided a compound according to formula I, wherein C is independently selected from phenyl or a 6 membered heteroaromatic ring; p is 1 and R³ is OSO₂R⁶.

In another aspect of the present invention, there is provided a compound according to formula I, wherein R⁶ is C₁₋₆alkyl.

In another aspect of the present invention, there is provided a compound according to formula I, wherein R⁶ is trifluoromethyl.

In another aspect of the present invention, there is provided a compound according to formula I, wherein m is 0; n is 0; p is 0; and q is 1.

In another aspect of the present invention, there is provided a compound according to formula I, wherein m is 0; n is 1; p is 0; and q is 0.

In another aspect of the present invention, there is provided a compound according to formula I, wherein m is 0; n is 0; p is 1; and q is 0.

In another aspect of the present invention, there is provided a compound according to formula I, wherein t is 1 or 2.

In one embodiment of this aspect, R⁸ is located on C, and is independently selected from halogen, cyano, nitro and OR⁹.

In another embodiment of this aspect, R⁸ is located on C, and is independently selected from halogen, cyano, nitro and OR⁹, wherein R⁹ is selected from C₁₋₆alkyl and trifluoromethyl.

In another embodiment of this aspect, R⁸ is located on C, and is C₁₋₆alkyl optionally substituted with one or more E.

In another embodiment of this aspect, R⁸ is located on C, and is C₁₋₆alkyl, optionally substituted with one or more E, wherein E is halogen.

In another aspect of the present invention, there is provided a compound according to is formula I, wherein

A is selected from a 5 or 6 membered heterocyclic ring;

B is selected from phenyl or a 6 membered heteroaromatic ring optionally substituted with one R²;

C is selected from phenyl or a 6 membered heteroaromatic ring optionally substituted with one or more R³;

R² or R³ is OSO₂R⁶;

R⁵ is hydrogen;

R⁶ is C₁₋₆alkyl;

R⁸ is selected from halogen, cyano, nitro, OR⁹;

R⁹ is selected from C₁₋₆alkyl and trifluoromethyl;

m=0;

n=0 or 1;

p=0;

q=0, 1 or 2;

t=0 or 1;

wherein one of n or q is at least 1.

In another aspect of the present invention, there is provided a compound according to formula I, wherein

A is independently selected from a 5 or 6 membered heterocyclic ring;

B is phenyl, optionally substituted with one R²;

C is independently selected from phenyl or a 6 membered heteroaromatic ring optionally substituted with one or more R³;

R² or R³ is OSO₂R⁶;

R⁵ is hydrogen;

E is halogen;

R⁶ is independently selected from C₁₋₆alkyl and trifluoromethyl;

R⁸ is independently selected from halogen, OR⁹ and C₁₋₆alkyl, wherein said C₁₋₆alkyl is optionally substituted with one or more E;

R⁹ is independently selected from hydrogen and C₁₋₆alkyl;

m=0;

n=0 or 1;

p=0 or 1;

q=0;

t=Q, 1 or 2;

wherein one of n or p is at least 1.

In another aspect of the present invention, there is provided a compound according to formula I, wherein

A is a 6 membered heterocyclic ring substituted with two R¹;

B is phenyl substituted with one R²;

C is a 6 membered heteroaromatic ring;

R¹ is halogen;

R² is CONR⁶R⁷;

R⁸ is hydrogen;

R⁶ and R⁷ are C₁₋₆alkyl;

R⁸ is halogen;

m=2;

n=1;

p=0;

q=0; and

t=0 or 1.

In another aspect of the present invention, there is provided a compound according to formula I, selected from:
4-[6-Amino-8-(3′-methoxybiphenyl-3-yl)-2,3,4,8-tetrahydroimidazo[1,5-a]pyrimidin-8-yl]phenyl methanesulfonate acetate;
4-[6-Amino-8-(3-pyrazin-2-ylphenyl)-2,3,4,8-tetrahydroimidazo[1,5-a]pyrimidin-8-yl]phenyl methanesulfonate acetate;
4-{6-Amino-8-[3-(5-fluoropyridin-3-yl)phenyl]-2,3,4,8-tetrahydroimidazo[1,5-a]pyrimidin-8-yl}phenyl methanesulfonate acetate;
4-{6-Amino-8-[3-(5-methoxypyridin-3-yl)phenyl]-2,3,4,8-tetrahydroimidazo[1,5-a]pyrimidin-8-yl}phenyl methanesulfonate acetate;
4-[6-Amino-8-(3′-cyanobiphenyl-3-yl)-2,3,4,8-tetrahydroimidazo[1,5-a]pyrimidin-8-yl]phenyl methanesulfonate acetate;
4-[6-Amino-8-(3′-chlorobiphenyl-3-yl)-2,3,4,8-tetrahydroimidazo[1,5-a]pyrimidin-8-yl]phenyl methanesulfonate 0.25 acetate;
4-{6-Amino-8-[3-(6-fluoropyridin-3-yl)phenyl]-2,3,4,8-tetrahydroimidazo[1,5-a]pyrimidin-8-yl}phenyl methanesulfonate 0.25 acetate;
4-{6-Amino-8-[3-(2,6-difluoropyridin-3-yl)phenyl]-2,3,4,8-tetrahydroimidazo[1,5-a]pyrimidin-8-yl}phenyl methanesulfonate 0.25 acetate;
4-[6-Amino-8-(3-pyridin-3-ylphenyl)-2,3,4,8-tetrahydroimidazo[1,5-a]pyrimidin-8-yl]phenyl methanesulfonate 0.75 acetate;
4-{6-Amino-8-[3-(2-fluoropyridin-3-yl)phenyl]-2,3,4,8-tetrahydroimidazo[1,5-a]pyrimidin-8-yl}phenyl methanesulfonate 0.25 acetate;
4-{6-Amino-8-[3′-(trifluoromethoxy)biphenyl-3-yl]-2,3,4,8-tetrahydroimidazo[1,5-a]pyrimidin-8-yl}phenyl methanesulfonate 0.5 acetate;
4-[6-Amino-8-(2′-fluoro-3′-methoxybiphenyl-3-yl)-2,3,4,8-tetrahydroimidazo[1,5-a]pyrimidin-8-yl]phenyl methanesulfonate 0.5 acetate;
4-[6-Amino-8-(2′-fluoro-5′-methoxybiphenyl-3-yl)-2,3,4,8-tetrahydroimidazo[1,5-a]pyrimidin-8-yl]phenyl methanesulfonate 0.25 acetate;
4-[6-Amino-8-(3′-ethoxybiphenyl-3-yl)-2,3,4,8-tetrahydroimidazo[1,5-a]pyrimidin-8-yl]phenyl methanesulfonate 0.5 acetate;
4-[6-Amino-8-(3′-nitrobiphenyl-3-yl)-2,3,4,8-tetrahydroimidazo[1,5-a]pyrimidin-8-yl]phenyl methanesulfonate 0.5 acetate;
4-[6-Amino-8-(2′,5′-dimethoxybiphenyl-3-yl)-2,3,4,8-tetrahydroimidazo[1,5-a]pyrimidin-8-yl]phenyl methanesulfonate 0.5 acetate;
4-[6-Amino-8-(3′-cyano-4′-fluorobiphenyl-3-yl)-2,3,4,8-tetrahydroimidazo[1,5-a]pyrimidin-8-yl]phenyl methanesulfonate 0.5 acetate;
4-[6-Amino-8-(5′-cyano-2′-fluorobiphenyl-3-yl)-2,3,4,8-tetrahydroimidazo[1,5-a]pyrimidin-8-yl]phenyl methanesulfonate 0.75 acetate;
4-[6-Amino-8-(3-pyrimidin-5-ylphenyl)-2,3,4,8-tetrahydroimidazo[1,5-a]pyrimidin-8-yl]phenyl methanesulfonate 0.5 acetate;
4-[6-Amino-8-(3′,5′-dichlorobiphenyl-3-yl)-2,3,4,8-tetrahydroimidazo[1,5-a]pyrimidin-8-yl]phenyl methanesulfonate acetate;
3-{6-Amino-8-[3-(5-chloro-2-fluoropyridin-3-yl)phenyl]-2,3,4,8-tetrahydroimidazo[1,5-a]pyrimidin-8-yl}phenyl methanesulfonate
3′-[6-Amino-8-(4-methoxyphenyl)-2,3,4,8-tetrahydroimidazo[1,5-a]pyrimidin-8-yl]-5-methoxybiphenyl-3-yl methanesulfonate acetate;
3′-[6-Amino-8-(4-methoxyphenyl)-2,3,4,8-tetrahydroimidazo[1,5-a]pyrimidin-8-yl]-5-chlorobiphenyl-3-yl methanesulfonate acetate;
4-[6-Amino-8-(3-pyrazin-2-ylphenyl)-2,3,4,8-tetrahydroimidazo[1,5-a]pyrimidin-8-yl]phenyl propane-1-sulfonate acetate;
4-[6-Amino-8-(3′-methoxybiphenyl-3-yl)-2,3,4,8-tetrahydroimidazo[1,5-a]pyrimidin-8-yl]phenyl propane-1-sulfonate 0.75 acetate;
4-[6-Amino-8-(3′,5′-dichlorobiphenyl-3-yl)-2,3,4,8-tetrahydroimidazo[1,5-a]pyrimidin-8-yl]phenyl propane-1-sulfonate 0.5 acetate;
4-[6-Amino-8-(3′-chlorobiphenyl-3-yl)-2,3,4,8-tetrahydroimidazo[1,5-a]pyrimidin-8-yl]phenyl propane-1-sulfonate 0.75 acetate;
4-[6-Amino-8-(3-pyridin-3-ylphenyl)-2,3,4,8-tetrahydroimidazo[1,5-a]pyrimidin-8-yl]phenyl propane-1-sulfonate 0.5 acetate;
4-{6-Amino-8-[3-(2-fluoropyridin-3-yl)phenyl]-2,3,4,8-tetrahydroimidazo[1,5-a]pyrimidin-8-yl}phenyl propane-1-sulfonate acetate;
4-{6-Amino-8-[3′-(trifluoromethyl)biphenyl-3-yl]-2,3,4,8-tetrahydroimidazo[1,5-a]pyrimidin-8-yl}phenyl propane-1-sulfonate 0.5 acetate;
4-[6-Amino-8-(4′-fluoro-3′-methoxybiphenyl-3-yl)-2,3,4,8-tetrahydroimidazo[1,5-a]pyrimidin-8-yl]phenyl propane-1-sulfonate 0.75 acetate;
4-[6-Amino-8-(3′-chloro-2′-fluorobiphenyl-3-yl)-2,3,4,8-tetrahydroimidazo[1,5-a]pyrimidin-8-yl]phenyl propane-1-sulfonate 0.75 acetate;
4-[6-Amino-8-(2′,5′-dichlorobiphenyl-3-yl)-2,3,4,8-tetrahydroimidazo[1,5-a]pyrimidin-8-yl]phenyl propane-1-sulfonate 0.75 acetate;
4-{6-Amino-8-[3-(5-methoxypyridin-3-yl)phenyl]-2,3,4,8-tetrahydroimidazo[1,5-a]pyrimidin-8-yl}phenyl propane-1-sulfonate 0.75 acetate;
4-[6-Amino-8-(3′-methoxybiphenyl-3-yl)-2,3,4,8-tetrahydroimidazo[1,5-a]pyrimidin-8-yl]phenyl cyclopropane sulfonate 0.75 acetate;
4-[6-Amino-8-(3′,5′-dichlorobiphenyl-3-yl)-2,3,4,8-tetrahydroimidazo[1,5-a]pyrimidin-8-yl]phenyl cyclopropanesulfonate 0.75 acetate;
4-[6-Amino-8-(3′-chlorobiphenyl-3-yl)-2,3,4,8-tetrahydroimidazo[1,5-a]pyrimidin-8-yl]phenyl cyclopropane sulfonate 0.75 acetate;
4-[6-Amino-8-(3-pyridin-3-ylphenyl)-2,3,4,8-tetrahydroimidazo[1,5-a]pyrimidin-8-yl]phenyl cyclopropane sulfonate 0.75 acetate;
4-{6-Amino-8-[3-(2-fluoropyridin-3-yl)phenyl]-2,3,4,8-tetrahydroimidazo[1,5-a]pyrimidin-8-yl}phenyl cyclopropane sulfonate 0.75 acetate;
4-{6-Amino-8-[3′-(trifluoromethyl)biphenyl-3-yl]-2,3,4,8-tetrahydroimidazo[1,5-a]pyrimidin-8-yl}phenyl cyclopropane sulfonate 0.75 acetate;
4-[6-Amino-8-(3′-chloro-2′-fluorobiphenyl-3-yl)-2,3,4,8-tetrahydroimidazo[1,5-a]pyrimidin-8-yl]phenyl cyclopropane sulfonate 0.75 acetate;
4-[6-Amino-8-(2′,5′-dichlorobiphenyl-3-yl)-2,3,4,8-tetrahydroimidazo[1,5-a]pyrimidin-8-yl]phenyl cyclopropane sulfonate 0.5 acetate;
3′-[5-Amino-7-(4-methoxyphenyl)-2,7-dihydro-3H-imidazo[1,5-a]imidazol-7-yl]-5-methoxybiphenyl-3-yl methanesulfonate acetate;
4-[5-Amino-7-(3′-methoxybiphenyl-3-yl)-2,7-dihydro-3H-imidazo[1,5-a]imidazol-7-yl]phenyl methanesulfonate 0.25 acetate;
4-[5-Amino-7-(3′,5′-dichlorobiphenyl-3-yl)-2,7-dihydro-3H-imidazo[1,5-a]imidazol-7-yl]phenyl methanesulfonate 0.25 acetate;
4-[5-Amino-7-(3′-chlorobiphenyl-3-yl)-2,7-dihydro-3H-imidazo[1,5-a]imidazol-7-yl]phenyl methanesulfonate 0.5 acetate;
4-[5-Amino-7-(3-pyridin-3-ylphenyl)-2,7-dihydro-3H-imidazo[1,5-a]imidazol-7-yl]phenyl methanesulfonate 0.5 acetate;
4-{5-Amino-7-[3-(2-fluoropyridin-3-yl)phenyl]-2,7-dihydro-3H-imidazo[1,5-a]imidazol-7-yl}phenyl methanesulfonate 0.5 acetate;
4-{5-Amino-7-[3-(5-chloro-2-fluoropyridin-3-yl)phenyl]-2,7-dihydro-3H-imidazo[1,5-a]imidazol-7-yl}phenyl methanesulfonate 0.5 acetate;
4-[5-Amino-7-(3′-methoxybiphenyl-3-yl)-2,7-dihydro-3H-imidazo[1,5-a]imidazol-7-yl]phenyl propane-2-sulfonate 0.5 acetate;
4-[5-Amino-7-(3′,5′-dichlorobiphenyl-3-yl)-2,7-dihydro-3H-imidazo[1,5-a]imidazol-7-yl]phenyl propane-2-sulfonate 0.5 acetate;
4-[5-Amino-7-(3′-chlorobiphenyl-3-yl)-2,7-dihydro-3H-imidazo[1,5-a]imidazol-7-yl]phenyl propane-2-sulfonate 0.5 acetate;
4-[5-Amino-7-(3-pyridin-3-ylphenyl)-2,7-dihydro-3H-imidazo[1,5-a]imidazol-7-yl]phenyl propane-2-sulfonate 0.75 acetate;
4-{5-Amino-7-[3-(2-fluoropyridin-3-yl)phenyl]-2,7-dihydro-3H-imidazo[1,5-a]imidazol-7-yl}phenyl propane-2-sulfonate 0.75 acetate;
4-{5-Amino-7-[3-(5-methoxypyridin-3-yl)phenyl]-2,7-dihydro-3H-imidazo[1,5-a]imidazol-7-yl}phenyl propane-2-sulfonate 0.5 acetate;
3′-(6-Amino-8-pyridin-4-yl-2,3,4,8-tetrahydroimidazo[1,5-a]pyrimidin-8-yl)-5-chlorobiphenyl-3-yl methanesulfonate 0.5 acetate; and
3′-(6-Amino-8-pyridin-4-yl-2,3,4,8-tetrahydroimidazo[1,5-a]pyrimidin-8-yl)-5-methoxybiphenyl-3-yl methanesulfonate 0.25 acetate.

In another aspect of the present invention, there is provided a compound according to formula I, selected from:
4-[5-Amino-7-(3′-chlorobiphenyl-3-yl)-2,7-dihydro-3H-imidazo[1,5-a]imidazol-7-yl]phenyl trifluoromethanesulfonate 0.75 acetate;
4-[6-Amino-8-(3′-chlorobiphenyl-3-yl)-2,3,4,8-tetrahydroimidazo[1,5-a]pyrimidin-8-yl]phenyl trifluoromethanesulfonate acetate;
3′-(6-Amino-8-phenyl-2,3,4,8-tetrahydroimidazo[1,5-a]pyrimidin-8-yl)-5-methoxybiphenyl-3-yl methanesulfonate hydrochloride;
3-{6-Amino-8-[3′,5′-bis(trifluoromethyl)biphenyl-3-yl]-2,3,4,8-tetrahydroimidazo[1,5-a]pyrimidin-8-yl}phenyl methanesulfonate;
3-[6-Amino-8-(3′-chlorobiphenyl-3-yl)-2,3,4,8-tetrahydroimidazo[1,5-a]pyrimidin-8-yl]phenyl trifluoromethanesulfonate;
3-[6-Amino-8-(3′,5′-dichlorobiphenyl-3-yl)-2,3,4,8-tetrahydroimidazo[1,5-a]pyrimidin-8-yl]phenyl methanesulfonate;
3-[6-Amino-8-(3′-chlorobiphenyl-3-yl)-2,3,4,8-tetrahydroimidazo[1,5-a]pyrimidin-8-yl]phenyl methanesulfonate;
3-[6-Amino-8-(3′-methoxybiphenyl-3-yl)-2,3,4,8-tetrahydroimidazo[1,5-a]pyrimidin-8-yl]phenyl methanesulfonate;
3-[6-Amino-8-(3-pyridin-3-ylphenyl)-2,3,4,8-tetrahydroimidazo[1,5-a]pyrimidin-8-yl]phenyl methanesulfonate;
3-[6-Amino-8-(3-pyrimidin-5-ylphenyl)-2,3,4,8-tetrahydroimidazo[1,5-a]pyrimidin-8-yl]phenyl methanesulfonate;
3-{6-Amino-8-[3-(5-chloro-2-fluoropyridin-3-yl)phenyl]-2,3,4,8-tetrahydroimidazo[1,5-a]pyrimidin-8-yl}phenyl methanesulfonate;
3-[6-Amino-8-(3′,5′-dimethylbiphenyl-3-yl)-2,3,4,8-tetrahydroimidazo[1,5-a]pyrimidin-8-yl]phenyl methanesulfonate;
3-[6-Amino-8-(3′,5′-dichlorobiphenyl-3-yl)-2,3,4,8-tetrahydroimidazo[1,5-a]pyrimidin-8-yl]phenyl propane-1-sulfonate;
3-[6-Amino-8-(3′-methoxybiphenyl-3-yl)-2,3,4,8-tetrahydroimidazo[1,5-a]pyrimidin-8-yl]phenyl propane-1-sulfonate;
3-[6-Amino-8-(3-pyrimidin-5-ylphenyl)-2,3,4,8-tetrahydroimidazo[1,5-a]pyrimidin-8-yl]phenyl propane-1-sulfonate;
3-[6-Amino-8-(3′,5′-dichlorobiphenyl-3-yl)-2,3,4,8-tetrahydroimidazo[1,5-a]pyrimidin-8-yl]phenyl cyclopropanesulfonate;
3-[6-Amino-8-(3′-methoxybiphenyl-3-yl)-2,3,4,8-tetrahydroimidazo[1,5-a]pyrimidin-8-yl]phenyl cyclopropanesulfonate;
3-[6-Amino-8-(3′,5′-dichlorobiphenyl-3-yl)-2,3,4,8-tetrahydroimidazo[1,5-a]pyrimidin-8-yl]phenyl trifluoromethanesulfonate;
3-[6-Amino-8-(3′-methoxybiphenyl-3-yl)-2,3,4,8-tetrahydroimidazo[1,5-a]pyrimidin-8-yl]phenyl trifluoromethanesulfonate;
3-[6-Amino-8-(3-pyridin-3-ylphenyl)-2,3,4,8-tetrahydroimidazo[1,5-a]pyrimidin-8-yl]phenyl trifluoromethanesulfonate;
3-{6-Amino-8-[3-(5-chloro-2-fluoropyridin-3-yl)phenyl]-2,3,4,8-tetrahydroimidazo[1,5-a]pyrimidin-8-yl}phenyl trifluoromethanesulfonate;
3-[6-Amino-8-(3-pyrimidin-5-ylphenyl)-2,3,4,8-tetrahydroimidazo[1,5-a]pyrimidin-8-yl]phenyl trifluoromethanesulfonate; and
3′-[6-Amino-8-(3-methoxyphenyl)-2,3,4,8-tetrahydroimidazo[1,5-a]pyrimidin-8-yl]-5-methoxybiphenyl-3-yl methanesulfonate.

In another aspect of the present invention, there is provided a compound according to formula I, selected from:
3-{6-Amino-3,3-difluoro-8-[3-(2-fluoropyridin-3-yl)phenyl]-2,3,4,8-tetrahydroimidazo[1,5-a]pyrimidin-8-yl}-N,N-dimethylbenzamide; and
4-{6-Amino-3,3-difluoro-8-[3-(2-fluoropyridin-3-yl)phenyl]-2,3,4,8-tetrahydroimidazo[1,5-a]pyrimidin-8-yl}-N,N-dimethylbenzamide.

Some compounds of formula I may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

Compounds of the invention can be used as medicaments. In some embodiments, the present invention provides compounds of formula I, or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides use of compounds of formula I or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula I, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula Ia or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors and a cognitive and/or memory enhancing agent. Cognitive enhancing agents, memory enhancing agents and choline esterase inhibitors includes, but not limited to, onepezil (Aricept), galantamine (Reminyl or Razadyne), rivastigmine (Exelon), tacrine (Cognex) and memantine (Namenda, Axura or Ebixa).

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the invention has been diagnosed with a particular disease or disorder, such as those described herein. In these cases, the mammal or human being treated is in need of such treatment. Diagnosis, however, need not be previously performed.

The present invention also includes pharmaceutical compositions which contain, as the active ingredient, one or more of the compounds of the invention herein together with at least one pharmaceutically acceptable carrier, diluent or excipient.

The definitions set forth in this application are intended to clarify terms used throughout this application. The term “herein” means the entire application.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used in this application, the term “optionally substituted,” means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is methyl (i.e., CH₃), then 3 hydrogens on the carbon atom can be replaced. Examples of such substituents include, but are not limited to: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, C₅₋₆aryl, OC₅₋₆aryl, C(═O)C₅₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, NC(═O)(C₅₋₆heterocyclyl)₂.

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₀₋₆ alkyl” denotes alkyl having 0, 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used alone or as a suffix or prefix is intended to include both branched and straight-chain alkyne containing aliphatic hydrocarbon groups having from 2 to 12 carbon atoms or if a specified number of carbon atoms is provided then that specific number would be intended. For example “C₂₋₆alkynyl” denotes alkynyl having 2, 3, 4, 5 or 6 carbon atoms. Examples of alkynyl include, but are not limited to, ethynyl, 1-propynyl, 2-propynyl, 3-butynyl, -pentynyl, hexynyl and 1-methylpent-2-ynyl.

As used herein, “aromatic” refers to hydrocarbonyl groups having one or more unsaturated carbon ring(s) having aromatic characters, (e.g. 4n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 4 to 12 carbons atoms.

As used herein, “cycloalkynyl” refers to ring-containing hydrocarbyl groups having at least one carbon-carbon triple bond in the ring, and having from 7 to 12 carbons atoms.

As used herein, “halo” or “halogen” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or is nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted by acetyl, formyl, methyl or mesyl; and a ring is optionally substituted by one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl.

As used herein, “heteroaryl” or “heteroaromatic” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms. In some embodiments, the heteroaryl or heteroaromatic group has 1 heteroatom.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “halo” or “halogen” refers to fluoro, chloro, bromo and iodo.

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric acid.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

As used herein, “tautomer” means other structural isomers that exist in equilibrium resulting from the migration of a hydrogen atom. For example, keto-enol tautomerism is where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to 2H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³J, ¹²⁴I, ¹²⁵J and ¹³¹I. The radionuclide that is incorporated in the instant radio-labeled compounds will depend on the specific application of that radio-labeled compound.

For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In some embodiments, the present invention provides a compound of formula I or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have beneficial effects in treatment of Alzheimer's Disease and other neurodegenerative diseases associated with elevated levels and/or deposition of Aβ peptide. Therefore, it is believed that the compounds of the present invention may be used for the treatment of Alzheimer disease and disease associated with dementia Hence, compounds of the present invention and their salts are expected to be active against age-related diseases such as Alzheimer, as well as other Aβ related pathologies such as Downs syndrome and β-amyloid angiopathy. It is expected that the compounds of the present invention would most likely be used as single agents but could also be used in combination with a broad range of cognition deficit enhancement agents.

Methods of Preparation

The present invention also relates to processes for preparing the compound of formula (I) as a free base or a pharmaceutically acceptable salt thereof. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in Protective Groups in Organic Synthesis by T. W. Greene, P. G. M Wutz, 3^{rd} Edition, Wiley-Interscience, New York, 1999. It is understood that microwaves can be used for the heating of reaction mixtures.

Preparation of Intermediates

The process, wherein A, B, C, D, E, R¹, R², R³, R⁴, R⁵R⁶, R⁷, R⁸, R⁹ and R¹⁰ unless otherwise specified, are as hereinbefore defined, comprises,

(i) diazotization of a compound of formula II, wherein C is aryl, to obtain a compound of formula III, wherein halo represents a halogen such as bromine or chloride.

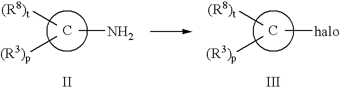

The reaction may be carried out by treating an appropriate amine with nitrous acid followed by treating the formed diazonium salt with an appropriate cuprous halide such as copper(I) bromide or copper(I) chloride, or with copper and hydrobromic acid or hydrochloric acid. The reactions may be preformed in a suitable solvent such as water at a temperature between −20° C. and reflux.

(ii) borylation of a compound of formula III, wherein halo represents halogen such as bromine or chlorine, to obtain a compound of formula IV, wherein R¹¹ represents hydrogen, alkyl, aryl or two R¹¹ may form a cyclic boronic ester.

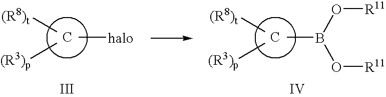

The reaction may be carried out by:

a) an alkyllithium such as butyllithium, or magnesium, and a suitable boron compound such as trimethyl borate or triisopropyl borate. The reaction may be performed in a suitable solvent such as tetrahydrofuran, hexane or dichloromethane in a temperature range between −78° C. and +20° C.; or,

b) a suitable boron species such as 4,4,4′,4′,5,5,5′,5′-octamethyl-2,2′-bi-1,3,2-dioxaborolane, biscatecholatodiboron, or pinacolborane in the presence of a suitable palladium catalyst such as tris(dibenzylideneaceton)dipalladium)(0), [1,1′-bis(diphenylphosphino)ferrocene]palladium(II) chloride, palladium(0) tetrakistriphenylphosphine, palladium diphenylphosphineferrocene dichloride or palladium acetate, with or without a suitable ligand such as tricyclohexylphosphine or 2-(dicyclohexylphosphino)biphenyl, and a suitable base, such as a tertiary amine, such as trietylamine or diisopropylethylamine, or potassium acetate may be used. The reaction may be performed in a solvent such as dioxane, toluene, acetonitrile, water, ethanol or 1,2-dimethoxyethane, or mixtures thereof, at temperatures between 20° C. and +160° C.

(iii) reaction of a compound of formula V wherein halo represents halogen e.g. bromide, R¹² is aryl or heteroaryl, and a compound of formula VI wherein R¹³ is aryl or heteroaryl, to obtain a compound of formula VII.

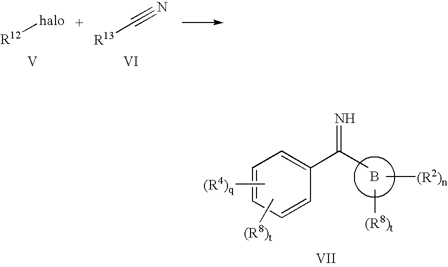

The reaction may be carried out by treating the compound of formula V with an alkyllithium, such as butyllithium, or magnesium followed by addition of a compound of formula VI. The reaction may be preformed in a suitable solvent such as diethyl ether or tetrahydrofuran at a temperature between −78° C. and reflux.

(iv) reaction of a compound of formula VII to obtain a compound of formula VIII.

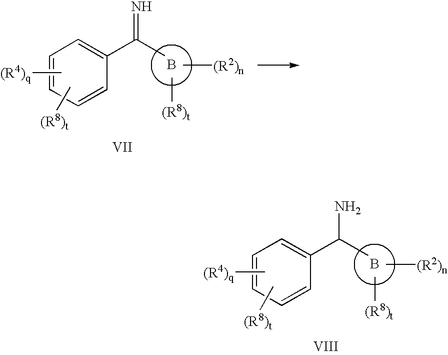

The reaction may be carried out by reduction using an appropriate reducing agent such as sodium borohydride, cyanoborohydride or lithium aluminium hydride. The reaction may be preformed in a suitable solvent such as methanol, ethanol, diethyl ether or tetrahydrofuran at a temperature between −78° C. and reflux.

(v) reaction of a compound of formula VIII to obtain a compound of formula IX.

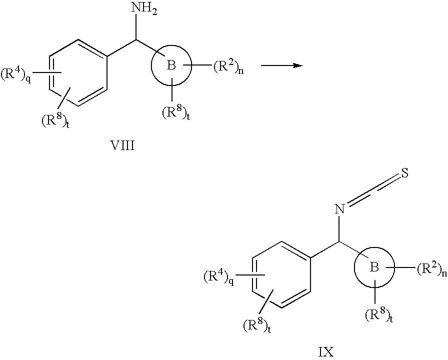

The reaction may be carried out by treating the compound of formula VIII with a suitable thiocarbonyl transfer reagent such as O,O-dipyridine-2-yl thiocarbonate or thiophosgene. The reaction may be preformed in a suitable solvent such as dichloromethane or chloroform at a temperature between −78° C. and reflux.

(vi) reaction of a compound of formula IX to obtain a compound of formula X.

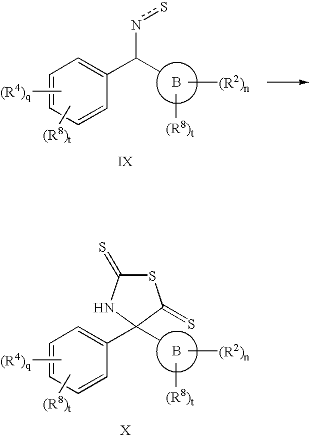

The reaction may be carried out by treating the appropriate isothiocyanate such as a compound of formula IX and carbon disulfide with a suitable base such as potassium tert-butoxide in a suitable solvent such as tetrahydrofuran or diethyl ether at a temperature between −78° C. and reflux.

(vii) reaction of a compound of formula X to obtain a compound of formula XI.

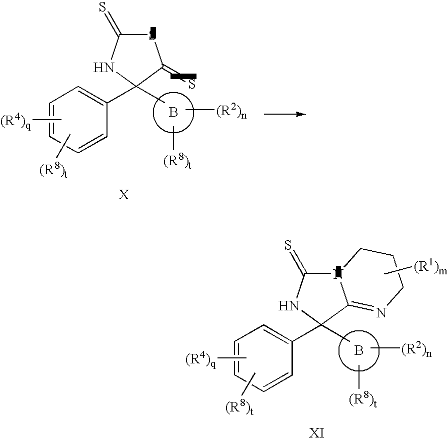

The reaction may be carried out by treating a compound of formula X with an appropriate diamine such as 1,3-diaminopropane or ethylenediamine. The reaction may be preformed in a suitable solvent such as ethanol or methanol at a temperature between 0° C. and reflux.

(viii) reaction of a compound of formula XII wherein F is ring B, C or phenyl in formula I, to a compound of formula XIII.

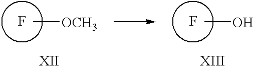

The reaction may be carried out by treating the methyl ether with a suitable Lewis acid such as boron tribromide in a suitable solvent such as dichloromethane at a temperature between −78° C. and reflux.

(ix) reaction of a compound of formula XIII, wherein F is ring B, C or phenyl in formula I, to a compound of formula XIV, wherein R¹⁴ is alkyl.

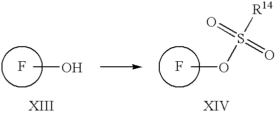

The reaction may be carried out by treating the appropriate alcohol with a suitable sulfonyl chloride or anhydride such as methanesulfonyl chloride, 1-propanesulfonyl chloride, cyclopropanesulfonyl chloride or methanesulfonic anhydride in the presence of a suitable base such as triethylamine. The reaction may be carried out in a suitable solvent such as dichloromethane at a temperature between 0° C. and reflux. Or the reaction may be carried out by treating the appropriate alcohol with 1,1,1-trifluoro-N-phenyl-N-[(trifluoromethyl)sulfonyl]methanesulfonamide in the presence of a suitable base such as potassium carbonate or N-ethyldiisopropylamine. The reaction may be carried out in a suitable solvent such as dichloromethane or tetrahydrofuran at a temperature between 0° C. and +160° C.

(x) reaction of a compound of formula XI to obtain a compound of formula XV.

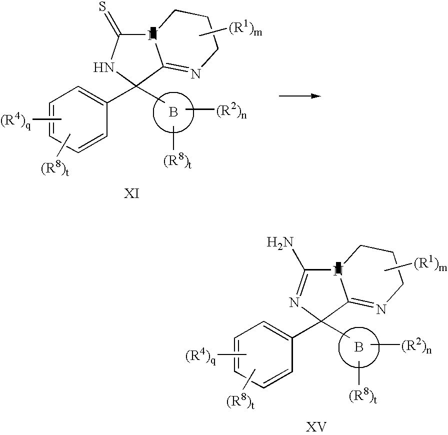

The reaction may be carried out by treating the appropriate thione such as a compound of formula XI with an appropriate oxidazing agent such as tert-butyl hydroperoxide and aqueous ammonia. The reaction may be performed in a suitable solvent such as methanol at a temperature between 0° C. and reflux.

Methods of Preparation of End Products

Another object of the invention is the processes a and b for the preparation of compounds of general Formula (a), wherein A, B, C, D, E, R¹, R², R³, R⁴, R⁵, R⁶, R⁷, R⁸, R⁹ and R¹⁰ unless otherwise specified, are defined as hereinbefore, and salts thereof. When it is desired to obtain the acid salt, the free base may be treated with an acid such as a hydrogen halide such as hydrogen chloride in a suitable solvent such as tetrahydrofuran, diethyl ether, methanol, ethanol, chloroform or dichloromethane or mixtures thereof and the reaction may occur between −30° C. to +50° C.

(a) reaction of a compound of formula XVI, wherein halo represents a halogen such as bromine, to obtain a compound of formula I.

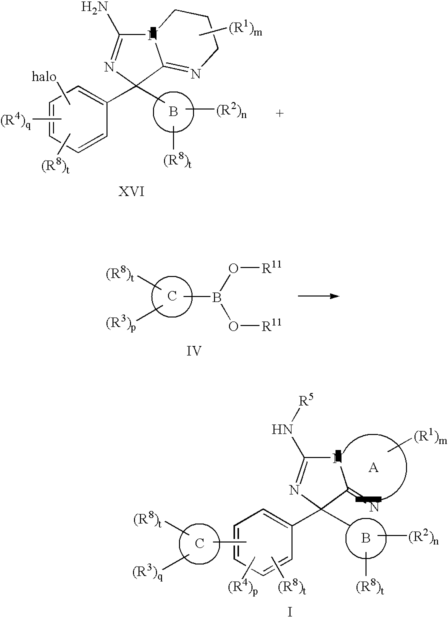

The reaction of process (a) may be carried out by coupling of a suitable compound such as a compound of formula XVI with an appropriate aryl boronic acid or ester of formula IV wherein R¹¹ represents hydrogen, alkyl, aryl or two R¹¹ may form a cyclic boronic ester. The reaction may be carried out using a suitable palladium catalyst such as, [1,1′-bis(diphenylphosphino)ferrocene]palladium(II) chloride, tetrakis(triphenylphosphine)palladium(0), palladium diphenylphosphineferrocene dichloride, palladium(II) acetate or bis(dibenzylideneacetone) palladium (0), together with, or without, a suitable ligand such as triphenylphosphine, tri-tert-butylphosphine or 2-(dicyclohexylphosphino)biphenyl, or using a nickel catalyst such as nickel on charcoal or 1,2-bis(diphenylphosphino)ethanenickel dichloride together with zinc and sodium triphenylphosphinetrimetasulfonate. A suitable base such as cesium fluoride, an alkyl amine such as triethyl amine, or an alkali metal or alkaline earth metal carbonate or hydroxide such as potassium carbonate, sodium carbonate, cesium carbonate, or sodium hydroxide may be used in the reaction, which may be performed in a suitable solvent such as toluene, tetrahydrofuran, dioxane, dimethoxyethane, water, ethanol or N,N-dimethylformamide, or mixtures thereof, at a temperature between +20° C. and +160° C.

(b) reaction of a compound of formula XVI, wherein halo represents a halogen such as bromine, to obtain a compound of formula I.

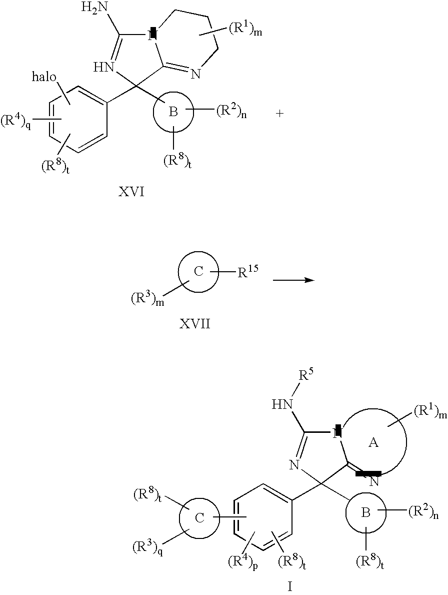

The reaction of process (b) may be carried out by coupling of a suitable compound such as a compound of formula XVI with an appropriate aryl stannane of formula XVII wherein R¹⁵ is trialkyltin. The reaction may be carried out using a suitable palladium catalyst such as, dichlorobis(triphenylphosphine)palladium(II), tetrakis(triphenylphosphine)palladium(0) or bis(dibenzylideneacetone) palladium (0), together with, or without, a suitable ligand such as triphenylarsine in a solvent such as N,N-dimethylformamide, tetrahydrofuran, toluene, 1-methyl-2-pyrrolidinone or dioxane at a temperature between +20° C. and reflux.

General Methods

Starting materials used were available from commercial sources, or prepared according to literature procedures.

Microwave heating was performed in a Creator, Initiator or Smith Synthesizer Single-mode microwave cavity producing continuous irradiation at 2450 MHz.

¹H NMR spectra were recorded in the indicated deuterated solvent at either 300 MHz, 400 MHz, 500 MHz, or 600 MHz. The 400 MHz spectra were obtained unless stated otherwise, using a Bruker av400 NMR spectrometer equipped with a 3 mm flow injection SEI ¹H/D-¹³C probe head with Z-gradients, using a BEST 215 liquid handler for sample injection, or using a Bruker DPX400 NMR spectrometer equipped with a 4-nucleus probehead with Z-gradients. 600 MHz ¹H NMR were recorded using a Bruker DRX 600 NMR spectrometer equipped with a 5 mm TXI probehead with Z-gradients. 500 MHz ¹H NMR were recorded using a Varian INOVA, (magnet: Oxford AS500) 500 NMR spectrometer. Chemical shifts are given in ppm down- and upfield from TMS. Resonance multiplicities are denoted s, d, t, q, m and br for singlet, doublet, triplet, quartet, multiplet, and broad respectively. LC-MS analyses were recorded on a Waters LCMS equipped with a Waters X-Terra MS, C8-column, (3.5 μm, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 2 mm ID×50 mm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm, or

LC-MS analyses were performed on an LC-MS system consisting of a Waters Alliance 2795 HPLC, a Waters PDA 2996 diode array detector, a Sedex 75 ELS detector and a ZMD single quadrupole mass spectrometer. The mass spectrometer was equipped with an electrospray ion source (ES) operated in positive or negative ion mode. The capillary voltage was set to 3.2 kV and the cone voltage to 30 V, respectively. The mass spectrometer was scanned between m/z 100-600 by a scan time of 0.7 s. The diode array detector was scanned from 200-400 nm. The temperature of the ELS detector was adjusted to 40° C. and the pressure was set to 1.9 bar. For separation a linear gradient was applied starting at 100% A (A: 10 mM ammonium acetate in 5% acetonitrile) and ending at 100% B (B: acetonitrile). The column used was an X-Terra MS C8, 3.0 mm×50 mm, 3.5 μm (Waters) run at a flow rate of 1.0 ml/min. The column oven temperature was set to 40° C. or

LC-MS analyses were performed on a LC-MS system consisting of a Waters Alliance 2795 HPLC, a Waters PDA 2996 diode array detector, a Sedex 75 ELS detector and a ZQ single quadrupole mass spectrometer. The mass spectrometer was equipped with an electrospray ion source (ES) operated in positive or negative ion mode. The capillary voltage was set to 3.2 kV and the cone voltage to 30 V, respectively. The mass spectrometer was scanned between m/z 100-700 with a scan time of 0.3 s. The diode array detector scanned from 200-400 nm. The temperature of the ELS detector was adjusted to 40° C. and the pressure was set to 1.9 bar. Separation was performed on an X-Terra MS C8, 3.0 mm×50 mm, 3.5 μm (Waters) run at a flow rate of 1 ml/min. A linear gradient was applied starting at 100% A (A: 10 mM ammonium acetate in 5% acetonitrile, or 8 mM formic acid in 5% acetonitrile) ending at 100% B (B: acetonitrile). The column oven temperature was set to 40° C. or

LC-MS analyses were performed on a LC-MS consisting of a Waters sample manager 2777C, a Waters 1525μ binary pump, a Waters 1500 column oven, a Waters ZQ single quadrupole mass spectrometer, a Waters PDA2996 diode array detector and a Sedex 85 ELS detector. The mass spectrometer was equipped with an electrospray ion source (ES) operated in positive or negative ion mode. The mass spectrometer scanned between m/z 100-700 with a scan time of 0.3 s. The capillary voltage was set to 3.4 kV and the cone voltage was set to 30 V, respectively. The diode array detector scanned from 200-400 nm. The temperature of the ELS detector was adjusted to 40° C. and the pressure was set to 1.9 bar. For separation a linear gradient was applied starting at 100% A (A: 10 mM ammonium acetate in 5% acetonitrile or 8 mM formic acid in 5% acetonitrile) and ending at 100% B (B: acetonitrile). The column used was a Gemini C18, 3.0 mm×50 mm, 3 μm, (Phenomenex) which was run at a flow rate of 1 ml/min. The column oven temperature was set to 40° C. or

LC-MS analyses were performed on a LC-MS system consisting of a Waters Alliance 2795 HPLC, a Waters PDA 2996 diode array detector, a Sedex 85 ELS detector and a ZQ single quadrupole mass spectrometer. The mass spectrometer was equipped with an electrospray ion source (ES) operated in positive or negative ion mode. The capillary voltage was set to 3.2 kV and the cone voltage to 30 V, respectively. The mass spectrometer scanned between m/z 100-700 with a scan time of 0.3 s. The diode array detector scanned from 200-400 nm. The temperature of the ELS detector was adjusted to 40° C. and the pressure was set to 1.9 bar. Separation was performed on an X-Terra MS C8, 3.0 mm×50 mm, 3.5 μm (Waters) run at a flow rate of 1 ml/min. A linear gradient was applied starting at 100% A (A: 10 mM ammonium acetate in 5% acetonitrile, or 8 mM formic acid in 5% acetonitrile) ending at 100% B (B: acetonitrile). The column oven temperature was set to 40° C. or LC-MS analyses were performed on a LC-MS consisting of a Waters sample manager 2777C, a Waters 1525μ binary pump, a Waters 1500 column oven, a Waters ZQ single quadrupole mass spectrometer, a Waters PDA2996 diode array detector and a Sedex 85 ELS detector. The mass spectrometer was configured with an atmospheric pressure chemical ionisation (APCI) ion source which was further equipped with atmospheric pressure photo ionisation (APPI) device. The mass spectrometer scanned in the positive mode, switching between APCI and APPI mode. The mass range was set to m/z 120-800 using a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode. Separation was performed using a Gemini column C18, 3.0 mm×50 mm, 3 μm, (Phenomenex) and run at a flow rate of 1 ml/min. A linear gradient was used starting at 100% A (A: 10 mM ammonium acetate in 5% methanol) and ending at 100% B (methanol). The column oven temperature was set to 40° C.

GC-MS: Compound identification was performed on a GC-MS system (GC 6890, 5973N MSD) supplied by Agilent Technologies. The column used was a VF-5 MS, ID 0.25 mm×15 m, 0.25 μm (Varian Inc.). A linear temperature gradient was applied starting at 40° C. (hold 1 min) and ending at 300° C. (hold 1 min), 25° C./minute. The mass spectrometer was equipped with a chemical ionisation (CI) ion source and the reactant gas was methane. The mass spectrometer was equipped with an electron impact (EI) ion source and the electron voltage was set to 70 eV. The mass spectrometer scanned between m/z 50-500 and the scan speed was set to 3.25 scan/s or

Compound identification was performed on a GC/DIP-MS system (GC 6890, 5973N MSD) supplied by Agilent Technologies. The mass spectrometer was equipped with a Direct Inlet Probe (DIP) interface manufactured by SIM GmbH. The mass spectrometer was configured with a chemical ionisation (CI) ion source and the reactant gas was methane. The mass spectrometer was equipped with an electron impact (EI) ion source and the electron voltage was set to 70 eV. The mass spectrometer scanned between m/z 50-500 and the scan speed was set to 3.25 scan/s. When introduced by direct inlet, 1 μl of the sample solution was added to the probe tip and a temperature gradient was applied to the probe starting at 40° C. and ending at 400° C., 2° C./s. When introduced by GC, a linear temperature gradient was applied starting at 40° C. (hold 1 min) and ending at 300° C. (hold 1 min), 25° C./minute. The column used was a VF-5 MS, ID 0.25 mm×30 m, 0.25 μm (Varian Inc.).

Preparative HPLC: Preparative chromatography was run on Waters auto purification HPLC with a diode array detector. Column: XTerra MS C8, 19×300 mm, 10 μm. Gradient with acetonitrile/0.1M ammonium acetate in 5% acetonitrile in MilliQ Water, run from 20% to 60% acetonitrile, in 13 min. Flow rate: 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Gradient with acetonitrile/0.1% trifluoroacetic acid in MilliQ Water, run from 35% to 60% acetonitrile in 20 min. Flow rate: 10 ml/min.

Alternatively, another column was used; Atlantis C18 19×100 mm, 5 μm column. Gradient with acetonitrile/0.1M ammonium acetate in 5% acetonitrile in MilliQ Water, run from 0% to 35-50% acetonitrile, in 15 min. Flow rate: 15 ml/min, or Preparative chromatography was run on a Waters FractionLynx system with a Autosampler combined Automated Fraction Collector (Waters 2767), Gradient Pump (Waters 2525), Regeneration Pump (Waters 600), Make Up Pump (Waters 515), Waters Active Splitter, Column Switch (Waters CFO), PDA (Waters 2996) and Waters ZQ mass spectrometer. Column; XBridge™ Prep C8 5 μm OBD™ 19×100 mm, with guard column; XTerra® Prep MS C8 10 μm 19×10 mm Cartridge. A gradient from 100% A (95% 0.1M ammonium acetate in MilliQ water and 5% acetonitrile) to 100% B (100% acetonitrile) was applied for LC-separation at flow rate 25 ml/min. The PDA was scanned from 210-350 nm. The ZQ mass spectrometer was run with ESI in positive mode. The Capillary Voltage was 3 kV and the Cone Voltage was 30V. Mixed triggering, UV and MS signal, determined the fraction collection.

Thin layer chromatography (TLC) was performed on Merch TLC-plates (Silica gel 60 F₂₅₄) and spots were UV visualized. Flash chromatography was performed using Merck Silica gel 60 (0.040-0.063 mm), or employing a Combi Flash® Companion™ system using RediSep™ normal-phase flash columns.

Compounds have been named using ACD/Name, version 8.0 or 9.0, software from Advanced Chemistry Development, Inc. (ACD/Labs), Toronto ON, Canada, www.acdlabs.com, 2004 and 2005.

### EXAMPLES

Below follows a number of non-limiting examples of compounds of the invention.

### Example 1

### 4-Bromo-1-fluoro-2-methoxybenzene

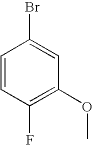

Aqueous hydrobromic acid (48%, 2.41 mL) was added to 4-fluoro-3-methoxyaniline (1.0 g, 7.1 mmol) in water (10 mL) and the resulting mixture was cooled to 0° C. in an ice bath. A solution of sodium nitrite (538 mg, 7.8 mmol) in water (5 mL) was added drop wise during 15 min while maintaining the temperature between 0-5° C. The resulting diazonium salt solution was added to a suspension of copper (J) bromide (1.12 g, 7.8 mmol) in water (5 mL) which had been pre-heated to 75° C. The mixture was shaken thoroughly, aqueous hydrobromic acid (48%, 12.07 mL) was added and the solution was stirred at ambient temperature for 16 h. Excess water was added and the product was extracted with diethyl ether and the combined organic extracts were washed with aqueous saturated sodium chloride, dried over magnesium sulfate, filtered and the solvent was evaporated in vacuo to give 1.02 g (70% yield) of the title compound: ¹H-NMR (DMSO-d₆): δ 7.36 (dd, J=7.78, 2.26 Hz, 1H), 7.23-7.17 (m, 1H), 7.14-7.09 (m, 1H), 3.86 (s, 3H); MS (EI) m/z 204, 206 [M+•]

### Example 2

### 2-(4-Fluoro-3-methoxyphenyl)-4,4,5,5-tetramethyl-1,3,2-dioxaborolane

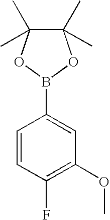

Anhydrous 1,2-dimethoxyethane (12 mL) was added to 4-bromo-1-fluoro-2-methoxybenzene (1.02 g, 5.0 mmol), tris(dibenzylideneaceton)dipalladium (0) (228 mg, 0.25 mmol), tricyclohexylphosphine (209 mg, 0.75 mmol), potassium acetate (732 mg, 7.5 mmol) and 4,4,4′,4′,5,5,5′,5′-octamethyl-2,2′-bi-1,3,2-dioxaborolane (1.14 g, 4.5 mmol) and the resulting mixture was irradiated in a microwave at 150° C. for 1 h. When cooled to ambient temperature the mixture was filtered and the solvent was evaporated in vacuo to give the crude product: MS (EI) m/z 252 [M+•]

### Example 3

### 3-Methoxy-5-(4,4,5,5-tetramethyl-1,3,2-dioxaborolan-2-yl)phenol

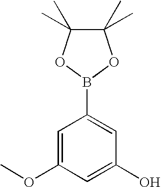

The title compound was synthesized as described for example 2 in 48% yield starting from 3-chloro-5-methoxyphenol. Purified by column chromatography, using a gradient of dichloromethane/acetonitrile (100/0 to 90/10) as the eluent: ¹H-NMR (DMSO-d₆): δ 9.36 (s, 1H), 6.69 (d, J=2.3 Hz, 1H), 6.61 (d, J=2.0 Hz, 1H), 6.41 (t, J=2.4 Hz, 1H), 3.69 (s, 3H), 1.27 (s, 12H); MS (ES) m/z 251 [M+1]⁺.

### Example 4

### 3-Methoxy-5-(4,4,5,5-tetramethyl-1,3,2-dioxaborolan-2-yl)phenyl methanesulfonate

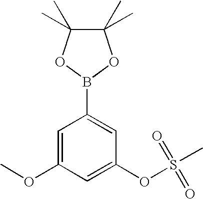

To a stirred solution of 3-methoxy-5-(4,4,5,5-tetramethyl-1,3,2-dioxaborolan-2-yl)phenol (0.12 g, 0.48 mmol) in dichloromethane (3 mL) was added triethylamine (0.058 g, 0.58 mmol) followed by methanesulfonyl chloride (0.071 g, 0.62 mmol) at 0° C. under an atmosphere of argon. The reaction mixture was allowed to reach ambient temperature and stirred for 18 h and the resulting mixture was concentrated to dryness in vacuo. Purified by column chromatography, using a gradient of dichloromethane/acetonitrile (100/0 to 90/10) as the eluent, to give 0.050 g (32% yield) of the title compound: ¹H-NMR (CDCl₃): δ 7.30 (d, J=2.3 Hz, 1H), 7.28 (d, J=2.0 Hz, 1H), 6.96 (t, J=2.4 Hz, 1H), 3.86 (s, 3H), 3.16 (s, 3H), 1.35 (s, 12H); MS (ES) m/z 329 [M+1]⁺.

### Example 5

### 3-Chloro-5-(4,4,5,5-tetramethyl-1,3,2-dioxaborolan-2-yl)phenol

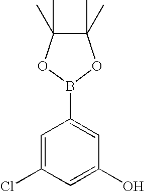

3-Bromo-5-chlorophenol (5 g, 19.9 mmol, described in: Maleczka R. E. et. al. J. Am. Chem. Soc. 2003, 125, 7792-7793), 4,4,4′,4′,5,5,5′,5′-octamethyl-2,2′-bi-1,3,2-dioxaborolane (6.06 g, 23.9 mmol), [1,1′-bis(diphenylphosphino)ferrocene]palladium(II) chloride dichloromethane adduct (487 mg, 0.6 mmol), potassium acetate (5.86 g, 59.7 mmol), 1,2-dimethoxyethane (60 mL) and water (4 mL) were divided into four microwave vials and irradiated in a microwave at 150° C. for 15 min each. When cooled to ambient temperature the mixtures were pooled, diluted with brine and extracted with diethyl ether. The combined organic phases were dried over sodium sulfate and concentrated in vacuo. Purified by column chromatography, using a gradient with 0-5% acetonitrile in dichloromethane as the eluent, to give 1.43 g (28% yield) of the title compound: ¹H NMR (DMSO-d₆) δ 9.89 (s, 1H), 7.02 (s, 2H), 6.91 (s, 1H), 1.28 (s, 12H); MS (ES) m/z 253 [M−1]⁻.

### Example 6

### 3-Chloro-5-(4,4,5,5-tetramethyl-1,3,2-dioxaborolan-2-yl)phenyl methanesulfonate

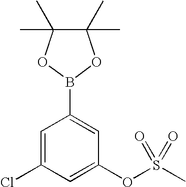

Methanesulfonyl chloride (122 μL, 0.79 mmol) was added dropwise at 0° C. to a mixture of 3-chloro-5-(4,4,5,5-tetramethyl-1,3,2-dioxaborolan-2-yl)phenol (200 mg, 0.79 mmol) and triethylamine (0.4 mL, 3.14 mmol) in dry dichloromethane (1.5 mL). The reaction mixture was stirred for 1 h at ambient temperature, diluted with dichloromethane (10 mL), washed with water, dried over sodium sulfate and concentrated in vacuo to give 0.200 g (86% yield) of the crude title compound: ¹H NMR (CDCl₃) δ 7.75 (d, J=1.52 Hz, 2H), 7.57 (d, J=1.77 Hz, 2H), 7.41 (t, J=2.15 Hz, 1H), 3.18 (s, 3H), 1.35 (s, 12H); GC-MS (EI) m/z 332 [M]⁺.

### Example 7

### 1-(3-Bromophenyl)-1-(4-methoxyphenyl)methanamine

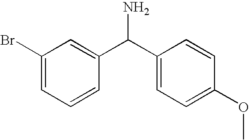

4-Bromoanisole (5.3 g, 28.4 mmol) in dry tetrahydrofuran (25 mL) was added dropwise to a mixture of magnesium (0.69 g, 28.4 mmol) and a crystal of iodide in dry tetrahydrofuran (25 mL) at 50° C. The mixture was stirred for 5 h and then cooled to room temperature. 3-Bromobenzonitrile (3.5 g, 19 mmol) in dry tetrahydrofuran (30 mL) was added dropwise over 30 min and the mixture was heated at 60° C. for 16 h. The mixture was cooled to room temperature and dry methanol (25 mL) was added and the mixture was stirred for another 45 min. The mixture was cooled to 0° C. and sodium borohydride (1.4 g, 38 mmol) was added in portions over 15 min, the mixture was then allowed to reach room temperature and stirred for 4 h. Saturated aqueous ammonium chloride was added and most of the organic solvents were removed in vacuo. The residue was extracted with dichloromethane. The organics were dried over sodium sulfate, filtrated and evaporated. Purification by column chromatography, using ethyl acetate from 10-35% in n-heptane as the eluent, afforded 4.5 g (81% yield) of the title compound: ¹H NMR (DMSO-d₆) δ 7.59-7.57 (m, 1H), 7.37-7.33 (m, 2H), 7.30-7.26 (m, 2H), 7.25-7.20 (m, 1H), 6.86-6.82 (m, 2H), 5.03 (s, 1H), 3.70 (s, 3H), 2.31 (br s, 2H); MS m/z (CI) 291, 293 [M+1]⁺.

### Example 8

### 1-(3-Bromophenyl)-1-pyridin-4-ylmethanamine

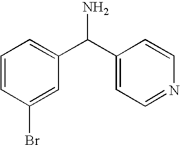

Butyllithium (2.5 M in hexanes, 10.20 mL, 25.40 mmol) was added to a cooled (−78° C.) solution of 1,3-dibromo-benzene (6 g, 25.40 mmol) in dry diethyl ether (60 mL), under an atmosphere of argon. The obtained mixture was stirred for 1 h at −78° C. 4-Cyanopyridine (2.64 g, 25.4 mmol) in dry diethyl ether (45 mL) was added and the stirring was continued for 20 min at −78° C. The reaction mixture was allowed to attain ambient temperature and dry methanol (30 mL) was added and the resulting mixture was stirred for another 45 min. The solution was cooled to 0° C., sodium borohydride (1.3 g, 34.0 mmol) was added and the reaction stirred overnight at ambient temperature. Saturated aqueous ammonium chloride (40 mL) was carefully added and the mixture was concentrated. The aqueous phase was extracted twice with dichloromethane (40 mL), the organic layer was dried over sodium sulfate, concentrated in vacuo, and the product was purified by column chromatography, using chloroform:methanol gradient elution 0-10%, to give 4.22 g (63% yield) of the title compound: ¹H NMR (CDCl₃) δ 8.56 (add, J=4.55, 1.52 Hz, 2H), 7.54 (t, J=1.77 Hz, 1H), 7.40 (dt, J=7.83, 1.52 Hz, 1H), 7.33-7.24 (m, 3H), 7.20 (t, J=7.83 Hz, 1H), 5.15 (s, 1H), 1.78 (br s, 2H); MS (ESI) m/z 264, 266 [M+1]⁺.

### Example 9

### 1-Bromo-3-[isothiocyanato(4-methoxyphenyl)methyl]benzene

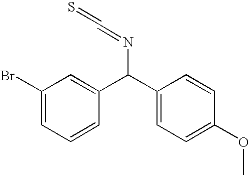

Thiophosgene (1.3 mL, 17 mmol) was added in portions to a stirred solution of 1-(3-bromophenyl)-1-(4-methoxyphenyl)methanamine (4.5 g, 15.4 mmol) in dichloromethane (70 mL) and saturated aqueous sodium bicarbonate (40 mL) at 0° C., and the mixture was stirred at 0° C. for 2 h. The organics were collected and the aqueous phase was extracted with dichloromethane. The combined organic extracts were washed with brine, dried over sodium sulfate, filtrated and concentrated in vacuo to give 5.02 g (98% yield) of the title compound: ¹H NMR (DMSO-d₆) δ 7.57-7.52 (m, 2H), 7.41-7.37 (m, 2H), 7.34-7.30 (m, 2H), 6.99-6.95 (m, 2H), 6.48 (s, 1H), 3.75 (s, 3H).

### Example 10

### 4-[(3-Bromophenyl)(isothiocyanato)methyl]pyridine

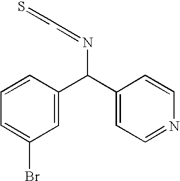

O,O-Dipyridin-2-yl thiocarbonate (183 mg, 0.79 mmol; described in: Kim S. et al. Tetrahedron Lett. 1985, 26(13), 1661-1664) was added, in one portion, to a solution of 1-(3-bromophenyl)-1-pyridin-4-ylmethanamine (100 mg, 0.38 mmol) in dichloromethane (2 mL). The mixture was stirred for 30 min and was then diluted with dichloromethane (15 mL), washed with brine, dried over sodium sulfate and concentrated in vacuo to give 0.100 g (86% yield) of the crude product: MS (ESI) m/z 305, 307 [M+1]⁺.

### Example 11

### 4-(3-Bromophenyl)-4-(4-methoxyphenyl)-1,3-thiazolidine-2,5-dithione

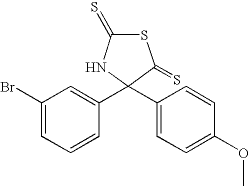

1-Bromo-3-[isothiocyanato(4-methoxyphenyl)methyl]benzene (8.7 g, 26 mmol) and carbon disulfide (3.1 mL, 52 mmol) in dry tetrahydrofuran (30 mL) was added dropwise to a stirred mixture of potassium tert-butoxide (4.2 g, 37 mmol) in dry tetrahydrofuran (80 mL) at −78° C. After the addition the mixture was allowed to reach room temperature overnight. Water, brine and ethyl acetate was added and the organic phase was collected. The aqueous phase was extracted with ethyl acetate and the combined organic extracts were washed with brine, dried over sodium sulfate and evaporated to give 10.5 g (98% yield) of the title product: ¹H NMR (DMSO-d₆) δ 7.48-7.43 (m, 1H), 7.41-7.39 (m, 1H), 7.31-7.24 (m, 2H), 7.22-7.18 (m, 2H), 6.89-6.85 (m, 2H), 3.74 (s, 3H).

### Example 12

### 4-(3-Bromo-phenyl)-4-pyridin-4-yl-thiazolidine-2,5-dithione

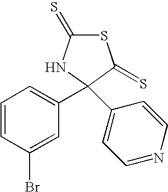

4-[(3-Bromophenyl)(isothiocyanato)methyl]pyridine (4.63 g, 15.19 mmol) and carbon disulfide (1.82 mL, 30.38 mmol) in dry tetrahydrofuran (30 mL) was added dropwise, at −78° C., to a stirred solution of potassium tert-butoxide (2.56 g, 22.79 mmol) in dry tetrahydrofuran (60 mL). The mixture was allowed to attain ambient temperature while stirring overnight. The solvent was evaporated and the residue dissolved in ethyl acetate (100 mL), washed with brine, dried over sodium sulfate and concentrated in vacuo. Purification by column chromatography, using chloroform:methanol 0-10% gradient elution, gave 4.95 g (85% yield) of the title compound: MS (ES) m/z 382, 383 [M+1]⁺.

### Example 13

### 8-(3-Bromophenyl)-8-(4-methoxyphenyl)-3,4,7,8-tetrahydroimidazo[1,5-a]pyrimidine-6(2H)-thione

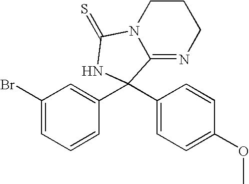

A solution of 4-(3-bromophenyl)-4-(4-methoxyphenyl)-1,3-thiazolidine-2,5-dithione (7 g, 15 mmol) and 1,3-diaminopropane (3.3 g, 44 mmol) in ethanol (70 mL) was heated at 70° C. for 1.5 h. The mixture was cooled to room temperature and concentrated, the residue was diluted with ethyl acetate, washed with saturated aqueous sodium bicarbonate and brine, dried over sodium sulfate and evaporated. Purification by column chromatography, using ethyl acetate from 5-25% in n-heptane as the eluent, afforded 5.1 g (83% yield) of the title compound: ¹H NMR (DMSO-d₆) δ 10.82 (s, 1H), 7.54-7.51 (m, 2H), 7.44-7.41 (m, 1H), 7.37-7.33 (m, 1H), 7.27-7.23 (m, 2H), 6.96-6.92 (m, 2H), 3.74 (s, 3H), 3.74-3.70 (m, 2H), 3.49-3.44 (m, 2H), 1.80-1.73 (m, 2H).

### Example 14

### 8-(3-Bromophenyl)-8-(4-hydroxyphenyl)-3,4,7,8-tetrahydroimidazo[1,5-a]pyrimidine-6(2H)-thione

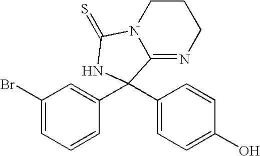

8-(3-Bromophenyl)-8-(4-methoxyphenyl)-3,4,7,8-tetrahydroimidazo[1,5-a]pyrimidine-6(2H)-thione (4.5 g, 10.9 mmol) was dissolved in dichloromethane (100 mL) and cooled to 0° C. Boron tribromide (1.5 mL, 16 mmol) was added and the mixture was warmed to room temperature and stirred for 2 h. The mixture was cooled to 0° C. and additional boron tribromide (1.5 mL, 16 mmol) was added, and the mixture was allowed to reach room temperature and stirred for 1 h. Water, brine and ethyl acetate were added and the organics were collected. The aqueous phase was treated with concentrated ammonium hydroxide and extracted with ethyl acetate. The combined organic phases were washed with brine, dried over sodium sulfate and evaporated to give 3.6 g (82% yield) of the title compound: ¹H NMR (DMSO-d₆) δ 10.77 (s, 1H), 9.58 (s, 1H), 7.55-7.51 (m, 2H), 7.44-7.40 (m, 1H), 7.38-7.32 (m, 1H), 7.13-7.09 (m, 2H), 6.76-6.72 (m, 2H), 3.74-3.69 (m, 2H), 3.48-3.43 (m, 2H), 1.80-1.73 (m, 2H).

### Example 15

### 4-[8-(3-Bromophenyl)-6-thioxo-2,3,4,6,7,8-hexahydroimidazo[1,5-a]pyrimidin-8-yl]phenyl methanesulfonate

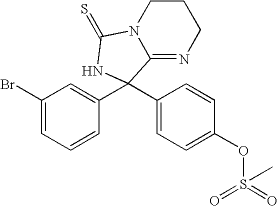

To a mixture of 8-(3-bromophenyl)-8-(4-hydroxyphenyl)-3,4,7,8-tetrahydroimidazo[1,5-a]pyrimidine-6(2H)-thione (3.6 g, 8.9 mmol) and triethylamine (1.7 mL, 12 mmol) in dichloromethane (50 mL) was added methanesulfonyl chloride (0.78 mL, 10 mmol) and the mixture was stirred overnight. Additional triethylamine (0.85 mL, 6 mmol) was added and the mixture was heated at 30° C. for 4 h. The solvent was evaporated and the residue was partitioned between ethyl acetate and saturated aqueous sodium bicarbonate. The organics were washed with brine, dried over sodium sulfate, and concentrated. Purification by column chromatography, using ethyl acetate in n-heptane from 10-50% as the eluent, afforded 2.4 g (56% yield) of the title compound: ¹H NMR (DMSO-d₆) δ 10.93 (br s, 1H), 7.59-7.51 (m, 2H), 7.48-7.43 (m, 3H), 7.40-7.35 (m, 3H), 3.76-3.71 (m, 2H), 3.51-3.47 (m, 2H), 3.40 (s, 3H), 1.82-1.75 (m, 2H).

### Example 16

### 4-[8-(3-Bromophenyl)-6-thioxo-2,3,4,6,7,8-hexahydroimidazo[1,5-a]pyrimidin-8-yl]phenyl propane-1-sulfonate

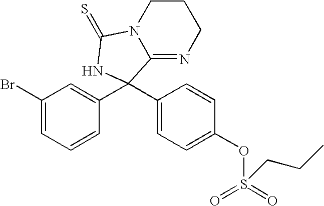

The title compound was prepared as described for example 15 in 68% yield starting from 8-(3-bromophenyl)-8-(4-hydroxyphenyl)-3,4,7,8-tetrahydroimidazo[1,5-a]pyrimidine-6(2H)-thione and 1-propanesulfonylchloride: ¹H NMR (DMSO-d₆) δ 10.93 (br s, 1H), 7.58-7.54 (m, 2H), 7.46-7.43 (m, 3H), 7.40-7.33 (m, 3H), 3.73 (t, J=5.90 Hz, 2H), 3.53-3.47 (m, 4H), 1.87-1.81 (m, 2H), 1.81-1.75 (m, 2H), 1.03 (t, J=7.53 Hz, 3H).

### Example 17

### 4-[8-(3-Bromophenyl)-6-thioxo-2,3,4,6,7,8-hexahydroimidazo[1,5-a]pyrimidin-8-yl]phenyl cyclopropanesulfonate

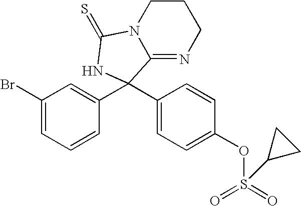

The title compound was prepared as described for example 15 in 65% yield starting from 8-(3-bromophenyl)-8-(4-hydroxyphenyl)-3,4,7,8-tetrahydroimidazo[1,5-a]pyrimidine-6(2H)-thione and cyclopropanesulfonyl chloride: ¹H NMR (DMSO-d₆) δ 10.93 (br s, 1H), 7.57-7.54 (m, 2H), 7.48-7.44 (m, 2H), 7.43-7.36 (m, 4H), 3.74 (t, J=6.15 Hz, 2H), 3.49 (t, J=5.27 Hz, 2H), 3.09-3.02 (m, 1H), 1.82-1.76 (m, 2H), 1.20-1.16 (m, 2H), 1.06-1.01 (m, 2H).

### Example 18

### 7-(3-Bromophenyl)-7-(4-methoxyphenyl)-2,3,6,7-tetrahydro-5H-imidazo[1,5-a]imidazole-5-thione

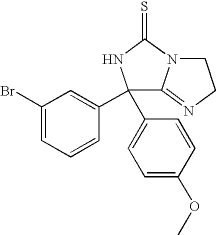

The title compound was prepared as described for example 13 in 55% yield starting from 4-(3-bromophenyl)-4-(4-methoxyphenyl)-1,3-thiazolidine-2,5-dithione and ethylenediamine (but heated for 36 h): ¹H NMR (DMSO-d₆) δ 10.62 (s, 1H), 7.62 (t, J=1.88 Hz, 1H), 7.57-7.54 (m, 1H), 7.49-7.46 (m, 1H), 7.38 (t, J=7.91 Hz, 1H), 7.33-7.29 (m, 2H), 6.99-6.95 (m, 2H), 4.31 (t, J=8.78 Hz, 2H), 3.74 (s, 3H), 3.71 (t, J=8.78 Hz, 2H).

### Example 19

### 7-(3-Bromophenyl)-7-(4-hydroxyphenyl)-2,3,6,7-tetrahydro-5H-imidazo[1,5-a]imidazole-5-thione

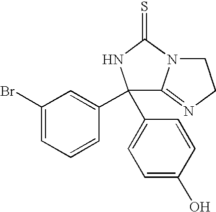

The compound was prepared as described for example 14 in 98% yield starting from 7-(3-bromophenyl)-7-(4-methoxyphenyl)-2,3,6,7-tetrahydro-5H-imidazo[1,5-a]imidazole-5-thione: ¹H NMR (DMSO-d₆) δ 10.56 (s, 1H), 9.64 (s, 1H), 7.63-7.61 (m, 1H), 7.56-7.53 (m, 1H), 7.49-7.45 (m, 1H), 7.38 (t, J=7.91 Hz, 1H), 7.20-7.16 (m, 2H), 6.79-6.75 (m, 2H), 4.30 (t, J=8.78 Hz, 2H), 3.70 (t, J=8.78 Hz, 2H).

### Example 20

### 4-[7-(3-Bromophenyl)-5-thioxo-2,5,6,7-tetrahydro-3H-imidazo[1,5-a]imidazol-7-yl]phenyl methanesulfonate

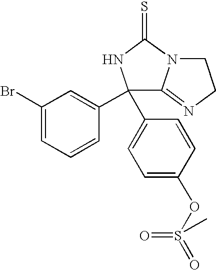

The compound was prepared as described for example 15 in 58% yield starting from 7-(3-bromophenyl)-7-(4-hydroxyphenyl)-2,3,6,7-tetrahydro-5H-imidazo[1,5-a]imidazole-5-thione: ¹H NMR (DMSO-d₆) δ 10.74 (s, 1H), 7.68 (t, J=1.88 Hz, 1H), 7.60-7.57 (m, 1H), 7.56-7.49 (m, 3H), 7.44-7.38 (m, 3H), 4.33 (t, J=9.03 Hz, 2H), 3.72 (t, J=8.91 Hz, 2H), 3.40 (s, 3H); MS (ES) m/z 464, 466 [M−1]⁻.

### Example 21

### 4-[7-(3-Bromophenyl)-5-thioxo-2,5,6,7-tetrahydro-3H-imidazo[1,5-a]imidazol-7-yl]phenyl propane-2-sulfonate

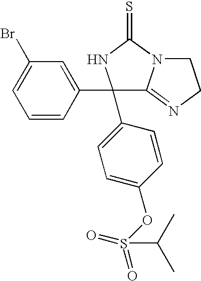

The compound was prepared as described for example 15 in 40% yield starting from 7-(3-bromophenyl)-7-(4-hydroxyphenyl)-2,3,6,7-tetrahydro-5H-imidazo[1,5-a]imidazole-5-thione and isopropylsulfonyl chloride: ¹H NMR (DMSO-d₆) δ 10.73 (s, 1H), 7.67 (t, J=1.76 Hz, 1H), 7.56-7.67 (m, 1H), 7.54-7.49 (m, 3H), 7.43-7.37 (m, 3H), 4.36-4.29 (m, 2H), 3.77-3.69 (m, 3H), 1.42 (s, 3H), 1.41 (s, 3H).

### Example 22

### 8-(3-Bromophenyl)-8-pyridin-4-yl-3,4,7,8-tetrahydroimidazo[1,5-a]pyrimidine-6(2H)-thione

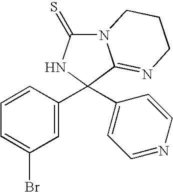

A mixture of 4-(3-bromo-phenyl)-4-pyridin-4-yl-thiazolidine-2,5-dithione (1.99 g, 5.22 mmol) and 1,3-diaminopropane (1.31 mL, 15.66 mmol) in ethanol (40 mL) was heated overnight at 70° C. The mixture was cooled to ambient temperature and concentrated in vacuo. The residue was dissolved in dichloromethane (40 mL), washed with water and brine, dried over sodium sulfate and concentrated in vacuo. Purification by column chromatography, using chloroform:methanol 0-10% gradient elution as the eluent, gave 1.59 g (79% yield) of the title compound: ¹H NMR (CDCl₃) δ 8.89 (s, 1H), 8.64 (dd, J=4.55, 1.77 Hz, 2H), 7.58-7.50 (m, 2H), 7.38-7.31 (m, 3H), 7.29-7.25 (m, 1H), 3.92 (t, J=6.06 Hz, 2H), 3.65 (q, J=5.64 Hz, 2H), 2.01-1.90 (m, 2H); MS (ES) m/z 387,389 [M+1]⁺.

### Example 23

### 4-[6-Amino-8-(3-bromophenyl)-2,3,4,8-tetrahydroimidazo[1,5-a]pyrimidin-8-yl]phenyl methanesulfonate

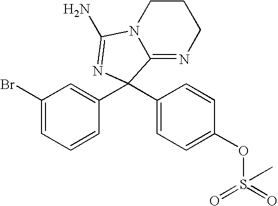

4-[8-(3-Bromophenyl)-6-thioxo-2,3,4,6,7,8-hexahydroimidazo[1,5-a]pyrimidin-8-yl]phenyl methanesulfonate (2.4 g, 5 mmol) was dissolved in methanol (70 mL) and concentrated ammonium hydroxide (40 mL). tert-Butyl hydroperoxide (13.7 mL, 70% in water, 100 mmol) was added and the mixture was stirred at room temperature overnight, and then heated at 30° C. for 3 h. Most of the methanol was evaporated, water and saturated aqueous sodium carbonate was added and the mixture was extracted with ethyl acetate. The combined organic extracts were washed with water and brine, dried over sodium sulfate and evaporated. Drying in a vacuum oven afforded 2.1 g (90% yield) of the title product: ¹H NMR (DMSO-d₆) δ 7.73-7.72 (m, 1H), 7.63-7.57 (m, 3H), 7.39-7.36 (m, 1H), 7.26-7.22 (m, 3H), 6.35 (br s, 2H), 3.55-3.51 (m, 2H), 3.43-3.39 (m, 2H), 3.34 (s, 3H), 1.71-1.65 (m, 2H).

### Example 24

### 4-[6-Amino-8-(3-bromophenyl)-2,3,4,8-tetrahydroimidazo[1,5-a]pyrimidin-8-yl]phenyl propane-1-sulfonate

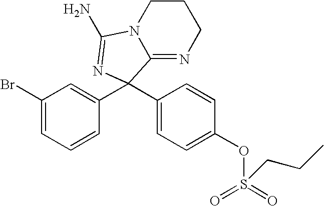

The title compound was prepared as described for example 23 in 102% yield starting from 4-[8-(3-bromophenyl)-6-thioxo-2,3,4,6,7,8-hexahydroimidazo[1,5-a]pyrimidin-8-yl]phenyl propane-1-sulfonate: ¹H NMR (DMSO-d₆) δ 7.72 (t, J=1.88 Hz, 1H), 7.63-7.56 (m, 3H), 7.40-7.36 (m, 1H), 7.27-7.19 (m, 3H), 3.53 (t, J=5.65 Hz, 2H), 3.49-3.44 (m, 2H), 3.42 (t, J=5.52 Hz, 2H), 1.86-1.77 (m, 2H), 1.72-1.65 (m, 2H), 1.02 (t, J=7.40 Hz, 3H); MS (ES) m/z 489, 491 [M−1]⁻.

### Example 25

### 4-[6-Amino-8-(3-bromophenyl)-2,3,4,8-tetrahydroimidazo[1,5-a]pyrimidin-8-yl]phenyl cyclopropanesulfonate

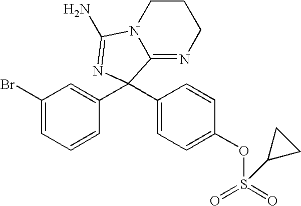

The title compound was prepared as described for example 23 in 97% yield starting from 4-[8-(3-bromophenyl)-6-thioxo-2,3,4,6,7,8-hexahydroimidazo[1,5-a]pyrimidin-8-yl]phenyl cyclopropanesulfonate: ¹H NMR (DMSO-d₆) δ 7.69 (t, J=1.88 Hz, 1H), 7.63-7.58 (m, 2H), 7.57-7.53 (m, 1H), 7.40-7.36 (m, 1H), 7.26-7.23 (m, 3H), 3.54 (t, J=5.77 Hz, 2H), 3.41 (t, J=5.40 Hz, 2H), 3.04-2.97 (m, 1H), 1.73-1.65 (m, 2H), 1.19-1.15 (m, 2H), 1.05-0.98 (m, 2H); MS (ES) m/z 487, 489 [M−1]⁻.

### Example 26

### 8-(3-Bromophenyl)-8-(4-methoxyphenyl)-2,3,4,8-tetrahadroimidazo[1,5-a]pyrimidin-6-amine

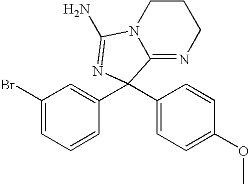

The title compound was prepared as described for example 23 in 99% yield starting from 8-(3-bromophenyl)-8-(4-methoxyphenyl)-3,4,7,8-tetrahydroimidazo[1,5-a]pyrimidine-6(2H)-thione: ¹H NMR (DMSO-d₆) δ 7.67-7.65 (m, 1H), 7.55-7.51 (m, 1H), 7.42-7.38 (m, 2H), 7.37-7.33 (m, 1H), 7.23-7.19 (m, 1H), 6.83-6.79 (m, 2H), 3.70 (s, 3H), 3.54-3.50 (m, 2H), 3.41-3.37 (m, 2H), 1.70-1.64 (m, 2H).

### Example 27

### 7-(3-Bromophenyl)-7-(4-methoxyphenyl)-2,7-dihydro-3H-imidazo[1,5-a]imidazol-5-amine

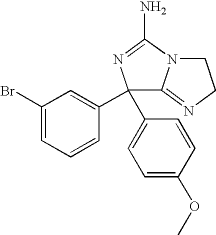

The title compound was prepared as described for example 23 in 97% yield starting from 7-(3-bromophenyl)-7-(4-methoxyphenyl)-2,3,6,7-tetrahydro-5H-imidazo[1,5-a]imidazole-5-thione: ¹H NMR (DMSO-d₆) δ 10.72 (br s, 2H), 7.70 (t, J=1.76 Hz, 1H), 7.56-7.52 (m, 1H), 7.45-7.40 (m, 2H), 7.39-7.36 (m, 1H), 7.24 (t, J=7.91 Hz, 1H), 6.86-6.82 (m, 2H), 4.29-4.22 (m, 2H), 3.70 (s, 3H), 3.53-3.47 (m, 2H).

### Example 28

### 4-[5-Amino-7-(3-bromophenyl)-2,7-dihydro-3H-imidazo[1,5-a]imidazol-7-yl]phenyl methanesulfonate

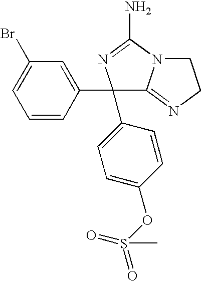

The title compound was prepared as described for example 23 in 99% yield starting from 4-[7-(3-bromophenyl)-5-thioxo-2,5,6,7-tetrahydro-3H-imidazo[1,5-a]imidazol-7-yl]phenyl methanesulfonate: ¹H NMR (DMSO-d₆) δ 10.73 (s, 1H), 7.76 (t, J=1.76 Hz, 1H), 7.67-7.63 (m, 2H), 7.60-7.57 (m, 1H), 7.42-7.39 (m, 1H), 7.29-7.27 (m, 3H), 4.29 (t, J=8.91 Hz, 2H), 3.52 (t, J=8.78 Hz, 2H), 3.35 (s, 3H). MS (ES) m/z 447, 449 [M−1]⁻.

### Example 29

### 4-[5-Amino-7-(3-bromophenyl)-2,7-dihydro-3H-imidazo[1,5-a]imidazol-7-yl]phenyl propane-2-sulfonate

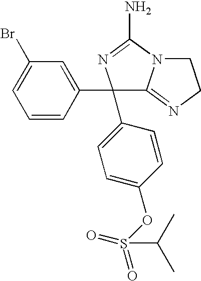

The title compound was prepared as described for example 23 in 106% yield starting from 4-[7-(3-bromophenyl)-5-thioxo-2,5,6,7-tetrahydro-3H-imidazo[1,5-a]imidazol-7-yl]phenyl propane-2-sulfonate: ¹H NMR (DMSO-d₆) δ 7.75 (t, J=1.88 Hz, 1H), 7.65-7.61 (m, 2 H), 7.59-7.56 (m, 1H), 7.42-7.39 (m, 1H), 7.29-7.23 (m, 3H), 6.47 (br s, 2H), 4.29 (t, J=8.78 Hz, 2H), 3.74-3.64 (m, 1H), 3.52 (t, J=8.78 Hz, 2H), 1.41 (s, 3H), 1.39 (s, 3H).

### Example 30

### 8-(3-Bromo-phenyl)-8-pyridin-4-yl-2,3,48-tetrahydro-imidazo[1,5-a]pyrimidin-6-ylamine

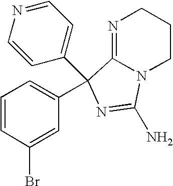

8-(3-Bromophenyl)-8-pyridin-4-yl-3,4,7,8-tetrahydroimidazo[1,5-a]pyrimidine-6(2H)-thione (2.60 g, 6.7 mmol) was dissolved in methanol (90 mL). Aqueous tert-butyl hydroperoxide (70%, 15 mL, 100.5 mmol) and aqueous ammonia (30%, 30 mL) were added and the resulting mixture was stirred overnight at ambient temperature. The mixture was concentrated and the residue was dissolved in dichloromethane (90 mL), washed with brine, dried over sodium sulfate and concentrated in vacuo. Purification by column chromatography, using chloroform (0.5% 7 M ammonia in methanol): methanol 0-10% gradient elution, gave 1.97 g (80% yield) of the title compound: ¹H NMR (CDCl₃) δ 8.52 (d, J=6.06 Hz, 2H), 7.66 (t, J=1.77 Hz, 1H), 7.46-7.36 (m, 4H), 7.16 (t, J=7.96 Hz, 1H), 3.72 (t, J=5.94 Hz, 2H), 3.61 (ddd, J=5.43, 2.65, 2.53 Hz, 2H), 1.92-1.82 (m, 2H); MS (ES) m/z 370, 372 [M+1]⁺.

Method A:

### Example 31

### 4-[6-Amino-8-(3′-methoxybiphenyl-3-yl)-2,3,4,8-tetrahydroimidazo[1,5-a]pyrimidin-8-yl]phenyl methanesulfonate acetate

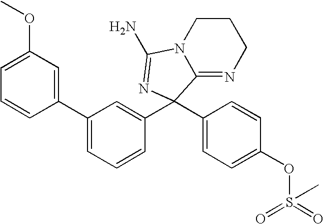

4-[6-Amino-8-(3-bromophenyl)-2,3,4,8-tetrahydroimidazo[1,5-a]pyrimidin-8-yl]phenyl methanesulfonate (70 mg, 0.15 mmol), [1,1′-bis(diphenylphosphino)ferrocene]palladium(II) chloride dichloromethane adduct (12 mg, 0.015 mmol), potassium carbonate (125 mg, 0.9 mmol), and 3-methoxyphenylboronic acid (29 mg, 0.19 mmol) in dry tetrahydrofuran (3 mL) was irradiated in a microwave at 130° C. for 2 h. When cooled to ambient temperature the mixture was filtered and dimethyl sulfoxide (800 μL) was added. The solution was concentrated in vacuo to remove the tetrahydrofuran and purified by preparative HPLC to give 19 mg (23% yield) of the title compound: ¹H NMR (CDCl₃) δ 7.85-7.82 (m, 1H), 7.70-7.66 (m, 2H), 7.58-7.54 (m, 1H), 7.49-7.45 (m, 1H), 7.39-7.32 (m, 2H), 7.26-7.22 (m, 2H), 7.11-7.08 (m, 1H), 7.05-7.03 (m, 1H), 6.95-6.91 (m, 1H), 3.80 (s, 3H), 3.57-3.52 (m, 2H), 3.45-3.41 (m, 2H), 3.34 (s, 3H), 1.91 (s, 3H), 1.73-1.66 (m, 2H); MS (ES) m/z 491 [M+1]⁺.

Method B:

### Example 32

### 4-[6-Amino-8-(3-pyrazin-2-ylphenyl)-2,3,4,8-tetrahydroimidazo[1,5-a]pyrimidin-8-yl]phenyl methanesulfonate acetate

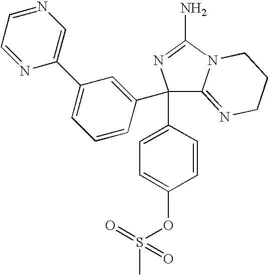

4-[6-Amino-8-(3-bromophenyl)-2,3,4,8-tetrahydroimidazo[1,5-a]pyrimidin-8-yl]phenyl methanesulfonate (60 mg, 0.13 mmol), dichlorobis(triphenylphosphine)palladium(II) (5 mg, 0.0065 mmol) and 2-tributylstannylpyranazine (67 mg, 0.18 mmol) in dry tetrahydrofuran (2 mL) was irradiated in a microwave at 130° C. for 1 h. When cooled to ambient temperature the mixture was filtered and dimethyl sulfoxide (800 μL) was added. The solution was concentrated in vacuo to remove tetrahydrofuran and purified by preparative HPLC to give 14 mg (23% yield) of the title compound. ¹H NMR (CDCl₃) δ 9.12 (d, J=1.51 Hz, 1H), 8.72-8.69 (m, 1H), 8.59 (d, J=2.51 Hz, 1H), 8.35 (t, J=1.63 Hz, 1H), 7.94-7.91 (m, 1H), 7.73 (d, J=8.03 Hz, 1H), 7.69-7.65 (m, 2H), 7.43 (t, J=7.78 Hz, 1H), 7.26-7.21 (m, 2H), 3.56-3.53 (m, 2H), 3.43-3.42 (m, 2H), 3.34 (s, 3H), 1.91 (s, 3H), 1.73-1.66 (m, 2H); MS (ES) m/z 463 [M+1]⁺.

### Examples 33-72

Examples 33-72 were synthesised as described for Method A (Example 31) or Method B (Example 32) in similar yields as exemplified in the Table below.

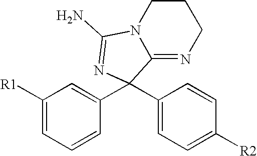

| | | | | | m/z | ¹H-NMR (DMSO-d₆) δ |
|---|---|---|---|---|---|---|
| Ex | Chemical name | R¹ | R² | Method | [M + 1]⁺ | ppm |
| 33 | 4-{6-Amino-8-[3- (5-fluoropyridin- 3-yl)phenyl]- 2,3,4,8- tetrahydroimidazo [1,5-a]pyrimidin- 8-yl}phenyl methanesulfonate acatete | 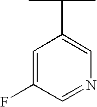 | CH₃SO₃ | A | 480 | |
| # 8.64 (t, J = 1.76 Hz, 1 H), 8.57 (d, J = 2.76 Hz, 1 H), 7.93 (t, J = 1.76 Hz, 1 H), 7.97-7.87 (m, 1 H), 7.69- 7.65 (m, 3 H), 7.60-7.57 (m, 1 H), 7.42 (t, J = 7.78 Hz, 1 H), 7.26-7.22 (m, 2 H), 3.57-3.53 (m, 2 H), 3.45-3.43 (m, 2 H), 3.34 (s, 3 H), 1.91 (s, 3H), 1.73- 1.67 (m, 2 H). | | | | | | |
| 34 | 4-{6-Amino-8-[3- (5- methoxypyridin-3- yl)phenyl]-2,3,4,8- tetrahydroimidazo [1,5-a]pyrimidin- 8-yl}phenyl methanesulfonate acetate | 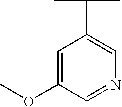 | CH₃SO₃ | A | 492 | |
| # 8.35-8.33 (m, 1 H), 8.28 (m, 1 H), 7.90-7.88 (m, 1 H), 7.70-7.66 (m, 2 H), 7.64-7.60 (m, 1 H), 7.57- 7.53 (m, 1 H), 7.47-7.45 (m, 1 H), 7.40 (t, 1 H), 7.26- 7.22 (m, 2 H), 3.89 (s, 3 H), 3.58-3.53 (m, 2 H), 3.45-3.42 (m, 2 H), 3.34 (s, 3 H), 1.91 (s, 3 H), 1.74 1.67 (m, 2 H). | | | | | | |
| 35 | 4-[6-Amino-8-(3′- cyanobiphenyl-3- yl)-2,3,4,8- tetrahydroimidazo [1,5-a]pyrimidin- 8-yl]phenyl methanesulfonate acetate | 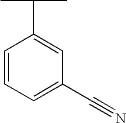 | CH₃SO₃ | A | 486 | |
| # 7.99-7.98 (m, 1 H), 7.91- 7.89 (m, 1 H), 7.88-7.85 (m, 1 H), 7.84-7.81 (m, 1H), 7.69-7.65 (m, 4 H), 7.57-7.54 (m, 1 H), 7.42- 7.38 (m, 1 H), 7.25-7.21 (m, 2 H), 3.57-3.52 (m, 2 H), 3.45-3.41 (m, 2 H), 3.34 (s, 3 H), 1.91 (s, 3 H), 1.73-1.67 (m, 2 H). | | | | | | |
| 36 | 4-[6-Amino-8-(3′- chlorobiphenyl-3- yl)-2,3,4,8- tetrahydroimidazo [1,5-a]pyrimidin- 8-yl]phenyl methanesulfonate 0.25 acetate | 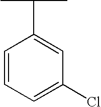 | CH₃SO₃ | A | 495/497 | |
| # 7.86 (t, J = 1.76 Hz, 1 H), 7.69-7.65 (m, 2 H), 7.64- 7.61 (m, 1 H), 7.57-7.55 (m, 1 H), 7.52-7.49 (m, 3 H), 7.44-7.41 (m, 1 H), 7.38 (t, J = 7.78 Hz, 1 H), 7.26-7.22 (m, 2 H), 3.57- 3.52 (m, 2 H), 3.45 -3.41 (m, 2 H), 3.34 (s, 3 H), 1.91 (s, 1.1 H), 1.72-1.67 (m, 2 H). | | | | | | |
| 37 | 4-{6-Amino-8-[3- (6-fluoropyridin- 3-yl)phenyl]- 2,3,4,8- tetrahydroimidazo [1,5-a]pyrimidin- 8-yl}phenyl methanesulfonate 0.25 acteate | 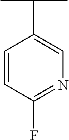 | CH₃SO₃ | A | 480 | |
| # 8.40 (d, J = 2.26 Hz, 1 H), 8.12 (td, J = 8.22, 2.64 Hz, 1 H), 7.86 (t, J = 1.76 Hz, 1 H), 7.69-7.65 (m, 2 H), 7.65-7.61 (m, 1 H), 7.54- 7.50 (m, 1 H), 7.40 (t, J = 7.65 Hz, 1 H), 7.28 (dd, J =8.53, 2.51 Hz, 1 H), 7.25- 7.22 (m, 2 H), 3.57-3.52 (m, 2 H), 3.45-3.41 (m, 2 H), 3.34 (s, 3 H), 1.91 (s, 0.6 H), 1.73-1.67 (m, 2 H). | | | | | | |
| 38 | 4-{6-Amino-8-[3- (2,6- difluoropyridin-3- yl)phenyl]-2,3,4,8- tetrahydroimidazo [1,5-a]pyrimidin- 8-yl}phenyl methanesulfonate 0.25 acetate | 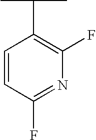 | CH₃SO₃ | A | 498 | |
| # 8.24-8.17 (m, 1 H), 7.82- 7.80 (m, 1 H), 7.69-7.62 (m, 3 H), 7.43-7.39 (m, 2 H), 7.28 (dd,J 8.28, 2.51 Hz, 1 H), 7.26-7.22 (m, 2 H), 3.56-3.52 (m, 2 H), 3.44-3.39 (m, 2 H), 3.34 (s, 3 H), 1.91 (s, 0.9 H), 1.72-1.66 (m, 2 H). | | | | | | |
| 39 | 4-[6-Amino-8-(3- pyridin-3- ylphenyl)-2,3,4,8- tetrahydroimidazo [1,5-a]pyrimidin- 8-yl]phenyl methanesulfonate 0.75 acetate | 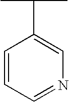 | CH₃SO₃ | A | 462 | |
| # 8.76-8.75 (m, 1 H), 8.56 (dd, J = 4.77, 1.51 Hz, 1 H), 7.94-7.91 (m, 1 H), 7.89 (t, J = 1.76 Hz, 1 H), 7.70-7.66 (m, 2 H), 7.64- 7.61 (m, 1 H), 7.55-7.52 (m, 1 H), 7.50-7.46 (m, 1 H), 7.40 (t, J = 7.65 Hz, 1 H), 7.26-7.22 (m, 2 H), | | | | | | |
| 40 | 4-{6-Amino-8-[3- (2-fluoropyridin- 3-yl)phenyl]- 2,3,4,8- tetrahydroimidazo [1,5-a]pyrimidin- 8-yl}phenyl methanesulfonate 0.25 acetate | 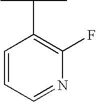 | CH₃SO₃ | A | 480 | |
| # 8.24-8.22 (m, 1 H), 8.03- 7.98 (m, 1 H), 7.85-7.82 (m, 1 H), 7.69-7.65 (m, 2 H), 7.65-7.62 (m, 1 H), 7.48-7.44 (m, 1 H), 7.43- 7.39 (m, 2 H), 7.26-7.22 (m, 2 H), 3.57-3.51 (m, 2 H), 3.44-3.39 (m, 2 H), 3.34 (s, 3 H), 1.91 (s, 0.8 H), 1.72-1.66 (m, 2 H). | | | | | | |
| 41 | 4-{6-Amino-8-[3′- (trifluoromethoxy) biphenyl-3-yl]- 2,3,4,8- tetrahydroimidazo [1,5-a]pyrimidin- 8-yl}phenyl methanesulfonate 0.5 acetate | 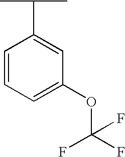 | CH₃SO₃ | A | 545 | |
| # 7.87 (t, J = 1.63 Hz, 1 H), 7.69-7.65 (m, 2 H), 7.63- 7.58 (m, 3 H), 7.54-7.50 (m, 1 H), 7.48-7.46 (m, 1 H), 7.41-7.34 (m, 2 H), 7.26-7.22 (m, 2 H), 3.57- 3.53 (m, 2 H), 3.45-3.41 (m, 2 H), 3.34 (s, 3 H), 1.91 (s, 1.5 H), 1.73-1.67 (m, 2 H). | | | | | | |
| 42 | 4-[6-Amino-8-(2′- fluoro-3′- methoxybiphenyl- tetrahydroimidazo [1,5-a]pyrimidin- 8-yl]phenyl methanesulfonate 0.5 acetate | 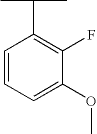 | CH₃SO₃ | A | 509 | |
| # 7.78-7.76 (m, 1 H), 7.71- 7.67 (m, 2 H), 7.60 (dt, J =7.09, 1.98 Hz, 1 H), 7.40- 7.33 (m, 2 H), 7.27-7.23 (m, 2 H), 7.22-7.15 (m, 2 H), 6.97-6.92 (m, 1 H), 3.88 (s, 3 H), 3.57-3.53 (m, 2 H), 3.44-3.40 (m, 2 H), 3.35 (s, 3 H), 1.91 (s, 1.5 H), 1.74-1.67 (m, 2 H). | | | | | | |
| 43 | 4-[6-Amino-8-(2′- fluoro-5′- methoxybiphenyl- tetrahydroimidazo [1,5-a]pyrimidin- 8-yl]phenyl methanesulfonate 0.25 acetate | 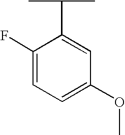 | CH₃SO₃ | A | 509 | |
| # 7.79-7.76 (m, 1 H), 7.70- 7.66 (m, 2 H), 7.60-7.56 (m, 1 H), 7.38-7.35 (m, 2 H), 7.26-7.18 (m, 3 H), 6.96-6.89 (m, 2 H), 3.77 (s, 3 H), 3.56-3.52 (m, 2 H), 3.43-3.39 (m, 2 H), 3.34 (s, 3 H), 1.91 (s, 1 H), 1.71-1.67 (m, 2 H). | | | | | | |
| 44 | 4-[6-Amino-8-(3′- ethoxybiphenyl-3- tetrahydroimidazo [1,5-a]pyrimidin- 8-yl]phenyl methanesulfonate 0.5 acetate | 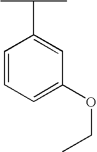 | CH₃SO₃ | A | 505 | |
| # 7.84-7.82 (m, 1 H), 7.70- 7.66 (m, 2 H), 7.58-7.54 (m, 1 H), 7.48-7.44 (m, 1 H), 7.37-7.31 (m, 2 H), 7.25-7.22 (m, 2 H), 7.09- 7.06 (m, 1 H), 7.03-7.01 (m, 1 H), 6.91 (dd, J = 8.41, 2.13 Hz, 1 H), 4.07 (q, J =7.03 Hz, 2 H), 3.57-3.52 (m, 2 H), 3.44-3.40 (m, 2 H), 3.34 (s, 3 H), 1.91 (s, 1.2 H), 1.72-1.67 (m, 2 H), 1.34 (t, J = | | | | | | |
| # 7.03 Hz, 3 H). | | | | | | |
| 45 | 4-[6-Amino-8-(3′- nitrobiphenyl-3- tetrahydroimidazo [1,5-a]pyrimidin- 8-yl]phenyl methanesulfonate 0.5 acetate | 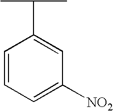 | CH₃SO₃ | A | 506 | |
| # 8.30 (t, J = 1.88 Hz, 1 H) 8.23-8.20 (m, 1 H), 8.03- 8.00 (m, 1 H), 7.94 (t, J =1.76 Hz, 1 H), 7.76 (t, J =8.03 Hz, 1 H), 7.71-7.65 (m, 3 H), 7.62-7.59 (m, 1 H), 7.43 (t, J = 7.78 Hz, 1 H), 7.26-7.22 (m, 2 H), 3.57-3.53 (m, 2 H) 3.46- 3.42 (m, 2 H) 3.34 (s, 3 H), 1.91 (s, 1.5 H), 1.73-1.68 (m, 2 H). | | | | | | |
| 46 | 4-[6-Amino-8- (2′,5′- dimethoxybiphenyl- 3-yl)-2,3,4,8- tetrahydroimidazo [1,5-a]pyrimidin- 8-yl]phenyl methanesulfonate 0.5 acetate | 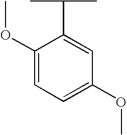 | CH₃SO₃ | A | 521 | |
| # 7.72-7.68 (m, 3 H), 7.50- 7.46 (m, 1 H), 7.30-7.26 (m, 2 H), 7.26-7.22 (m, 2 H), 7.00 (d, J = 8.78 Hz, 1 H), 6.88 (dd, J 9.03, 3.26 Hz, 1 H), 6.76-6.74 (m, 1 H), 3.72 (s, 3 H), 3.63 (s, 3 H), 3.56-3.52 (m, 2 H), 3.42-3.39 (m, 2 H), 3.34 (s, 3 H), 1.91 (s, 1.5 H), 1.72-1.66 (m, 2 H). | | | | | | |
| 47 | 4-[6-Amino-8-(3′- cyano-4′- fluorobiphenyl-3- tetrahydroimidazo [1,5-a]pyrimidin- 8-yl]phenyl methanesulfonate 0.5 acetate | 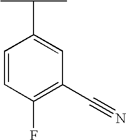 | CH₃SO₃ | A | 504 | |
| # 8.08 (dd, J = 6.02, 2.51 Hz, 1 H), 7.93-7.89 (m, 1 H), 7.87 (t, J = 1.76 Hz, 1 H), 7.68-7.59 (m, 4 H), 7.55- 7.51 (m, 1 H), 7.39 (t, J =7.78 Hz, 1 H), 7.25-7.21 (m, 2 H), 3.56-3.52 (m, 2 H), 3.45-3.41 (m, 2 H), 3.34 (s, 3 H), 1.91 (s, 1.5 H), 1.72-1.67 (m, 2 H). | | | | | | |
| 48 | 4-[6-Amino-8-(5′- cyano-2′- fluorobiphenyl-3- tetrahydroimidazo [1,5-a]pyrimidin- 8-yl]phenyl methanesulfonate 0.75 acetate | 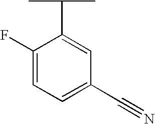 | CH₃SO₃ | A | 504 | |
| # 7.98 (dd, J = 7.28, 1 H), 7.95-7.91 (m, 1 H), 7.83- 7.81 (m, 1 H), 7.69-7.65 (m, 3 H), 7.57-7.51 (m, 1 H), 7.43-7.39 (m, 2 H), 7.25-7.21 (m, 2 H), 3.56- 3.52 (m, 2 H), 3.43-3.40 (m, 2 H), 3.34 (s, 3 H), 1.91 (s, 2.3 H), 1.72-1.67 (m, 2 H). | | | | | | |
| 49 | 4-[6-Amino-8-(3- pyrimidin-5- ylphenyl)-2,3,4,8- tetrahydroimidazo [1,5-a]pyrimidin- 8-yl]phenyl methanesulfonate 0.5 acetate | 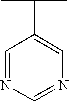 | CH₃SO₃ | A | 463 | |
| # 9.18 (s, 1 H), 9.00 (s, 2 H), 7.94 (t, J = 1.63 Hz, 1 H), 7.70-7.66 (m, 2 H), 7.63- 7.60 (m, 1 H), 7.44 (t, J =7.65 Hz, 1 H), 7.26-7.22 (m, 3 H), 3.57-3.53 (m, 2 H), 3.45-3.41 (m, 2 H), 3.34 (s, 3 H), 1.91 (s, 1.4 H), 1.72-1.67 (m, 2 H). | | | | | | |
| 50 | 4-[6-Amino-8- (3′,5′- dichlorobiphenyl- tetrahydroimidazo [1,5-a]pyrimidin- 8-yl]phenyl methanesulfonate acetate | 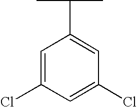 | CH₃SO₃ | A | 529/531 | |
| # 7.89-7.86 (m, 1 H), 7.70- 7.64 (m, 3 H), 7.62-7.603 H), 7.41-7.36 (m, 1 H), 7.25-7.22 (m, 2 H), 3.56- 3.51 (m, 2 H), 3.45-3.40 (m, 2 H), 3.34 (s, 3 H), 1.91 (s, 3 H), 1.72-1.66 (m, 2 H). | | | | | | |
| 51 | 3-{6-Amino-8-[3- (5-chloro-2- fluoropyridin-3- yl)phenyl]-2,3,4,8- tetrahydroimidazo [1,5-a]pyrimidin- 8-yl}phenyl methanesulfonate acetate | 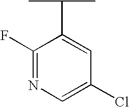 | CH₃SO₃ | A | 514/516 | |
| # 8.32-8.29 (m, 1 H), 8.18- 8.14 (m, 1 H), 7.88-7.85 (m, 1 H), 7.69-7.65 (m, 3 H), 7.48-7.39 (m, 2 H), 7.26-7.21 (m, 2 H), 3.54 (t, J = 5.65 Hz, 2 H), 3.43- 3.41 (m, 2 H), 3.34 (s, 3 H), 1.91 (s, 3 H), 1.72-1.66 (m, 2 H). | | | | | | |
| 52 | 3′-[6-Amino-8-(4- methoxyphenyl)- 2,3,4,8- tetrahydroimidazo [1,5-a]pyrimidin- methoxybiphenyl- 3-yl methanesulfonate acetate | 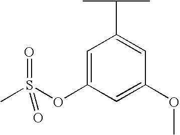 | CH₃O | A | 521 | |
| # 7.81-7.79 (m, 1 H), 7.58- 7.54 (m, 1 H), 7.51-7.47 (m, 1 H), 7.45-7.41 (m, 2 H), 7.38-7.33 (m, 1 H), 7.08-7.04 (m, 2 H), 6.95- 6.93 (m, 1 H), 6.83-6.79 (m, 2 H), 3.84 (s, 3 H), 3.69 (s, 3 H), 3.57-3.52 (m, 2 H), 3.42 (s, 3 H), 3.30-3.40 (m, 2 H), 1.91 (s, 3 H), 1.72-1.66 (m, 2 H). | | | | | | |
| 53 | 3′-[6-Amino-8-(4- methoxyphenyl)- 2,3,4,8- tetrahydroimidazo [1,5-a]pyrimidin- chlorobiphenyl-3- yl methanesulfonate acetate | 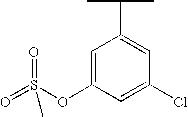 | CH₃O | A | 525/527 | |
| # 7.83 (t, J = 1.63 Hz, 1 H), 7.65-7.59 (m, 2 H), 7.55- 7.50 (m, 2 H), 7.47-7.36 (m, 4 H), 6.84-6.79 (m, 2 H), 3.69 (s, 3 H), 3.54 (t, J =5.90 Hz, 2 H), 3.47 (s, 3 H), 3.42-3.39 (m, 2 H), 1.91 (s, 3 H), 1.74- 1.65 (m, 2 H). | | | | | | |
| 54 | 4-[6-Amino-8-(3- pyrazin-2- ylphenyl)-2,3,4,8- tetrahydroimidazo [1,5-a]pyrimidin- 8-yl]phenyl propane-1- sulfonate acetate | 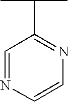 | CH₃CH₂CH₂SO₃ | B | 491 | |
| # 9.12 (d, J = 1.51 Hz, 1 H), 8.72-8.69 (m, 1 H), 8.59 (d, J = 2.51 Hz, 1 H), 8.35 (t, J = 1.76 Hz, 1 H), 7.95- 7.91 (m, 1 H), 7.75-7.71 (m, 1 H), 7.68-7.64 (m, 2 H), 7.43 (t, J = 7.78 Hz, 1 H), 7.23-7.19 (m, 2 H), 3.55 (t, J = 5.90 Hz, 2 H), 3.46-3.42 (m, 4 H), 1.91 (s, 3 H), 1.87- 1.77 (m, 2 H), 1.73-1.66 (m, 2 H), | | | | | | |
| # 1.01 (t, J = 7.40 Hz, 3 H). | | | | | | |
| 55 | 4-[6-Amino-8-(3′- methoxybiphenyl- 3-yl)-2,3,4,8- tetrahydroimidazo [1,5-a]pyrimidin- 8-yl]phenyl propane-1- sulfonate 0.75 acetate | 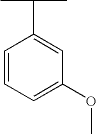 | CH₃CH₂CH₂SO₃ | A | 519 | |
| # 7.84-7.82 (m, 1 H), 7.69- 7.65 (m, 2 H), 7.57-7.54 (m, 1 H), 7.49-7.45 (m, 1 H), 7.39-7.32 (m, 2 H), 7.23-7.19 (m, 2 H), 7.11- 7.07 (m, 1 H), 7.05-7.03 (m, 1 H), 6.94-6.91 (m, 1 H), 3.80 (s, 3 H), 3.55 (t, J =5.90 Hz, 2 H), 3.47-3.43 (m, 4 H), 1.90 (s, 2.3 H), 1.85-1.78 (m, 2 H), 1.73- 1.67 (m, 2 H), 1.01 (t, J =7.40 Hz, 3 H). | | | | | | |
| 56 | 4-[6-Amino-8- (3,5′- dichiorobiphenyl- 3-yl)-2,3,4,8- tetrahydroimidazo [1,5-a]pyrimidin- 8-yl]phenyl propane-1- sulfonate 0.5 acetate | 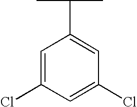 | CH₃CH₂CH₂SO₃ | A | 557/559 | |
| # 7.88-7.86 (m, 1 H), 7.69- 7.63 (m, 3 H), 7.60 (t, J =1.76 Hz, 1 H), 7.57-7.53 (m, 3 H), 7.39 (t, J = 7.65 Hz, 1 H), 7.23 -7.19 (m, 2 H), 3.54 (t, J = 5.77 Hz, 2 H), 3.47-3.42 (m, 4 H), 1.90 (s, 1.4 H), 1.85-1.78 (m, 2 H), 1.72-1.66 (m, 2 H), 1.01 (t, J = 7.53 Hz, 3 H). | | | | | | |
| 57 | 4-[6-Amino-8-(3′- chlorobiphenyl-3- yl)-2,3,4,8- tetrahydroimidazo [1,5-a]pyrimidin- 8-yl]phenyl propane-1- sulfonate 0.75 acetate | 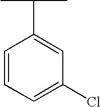 | CH₃CH₂CH₂SO₃ | A | 523/525 | |
| # 7.86-7.84 (m, 1 H), 7.68- 7.64 (m, 2 H), 7.64-7.61 (m, 1 H), 7.56-7.55 (m, 1 H), 7.52-7.48 (m, 3 H), 7.44-7.40 (m, 1 H), 7.37 (t, J = 7.78 Hz, 1 H), 7.23- 7.19 (m, 2 H), 3.55 (t, J =5.65 Hz, 2 H), 3.46-3.41 (m, 4 H), 1.90 (s, 2 H), 1.85- 1.78 (m, 2 H), 1.73-1.67 (m, 2 H), 1.01 (t, J = 7.53 Hz, 3 H). | | | | | | |
| 58 | 4-[6-Amino-8-(3- pyridin-3- ylphenyl)-2,3,4,8- tetrahydroimidazo [1,5-a]pyrimidin- 8-yl]phenyl propane-1- sulfonate 0.5 acetate | 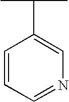 | CH₃CH₂CH₂SO₃ | A | 490 | 8.77 (d, J = 1.76 Hz, 1 H), 8.58 (dd, J = 4.64, 1.63 Hz, 1 H), 7.96-7.92 (m, 1 H), |
| # 7.90 (t, J = 1.63 Hz, 1 H), 7.71-7.67 (m, 2 H), 7.66- 7.62 (m, 1 H), 7.57-7.53 (m, 1 H), 7.52 -7.48 (m, 1 H), 7.42 (t, J = 7.65 Hz, 1 H), 7.24-7.20 (m, 2 H), 3.56 (t, J = 6.02 Hz, 2 H), 3.49-3.44 (m, 4 H), 1.91 (s, 1.5 H), 1.87-1.80 (m, 2 H), 1.74-1.68 (m, 2 H), 1.03 (t, J = 7.53 Hz, 3 H). | | | | | | |
| 59 | 4-{6-Amino-8-[3- (2-fluoropyridin- 3-yl)phenyl]- 2,3,4,8- tetrahydroimidazo [1,5-a]pyrimidin- 8-yl}phenyl propane-1- sulfonate acetate | 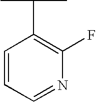 | CH₃CH₂CH₂SO₃ | A | 508 | |
| # 8.24-8.21 (m, 1 H), 8.03- 7.97 (m, 1 H), 7.84-7.81 (m, 1 H), 7.69-7.62 (m, 3 H), 7.48-7.38 (m, 3 H), 7.23-7.19 (m, 2 H), 3.54 (t, J = 5.77 Hz, 2 H), 3.46- 3.42 (m, 4 H), 1.90 (s, 2.7 H), 1.87-1.77 (m, 2 H), 1.72-1.66 (m, 2 H), 1.01 (t, J = 7.53 Hz, 3 H), | | | | | | |
| 60 | 4-{6-Amino-8-[3′- (trifluoroinethyl) biphenyl-3-yl]- 2,3,4,8- tetrahydroimidazo [1,5-a]pyrimidin- 8-yl}phenyl propane-1- sulfonate 0.5 acetate | 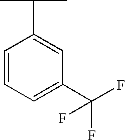 | CH₃CH₂CH₂SO₃ | A | 557 | |
| # 7.92-7.90 (m, 1 H), 7.88- 7.84 (m, 1 H), 7.83-7.81 (m, 1 H), 7.76-7.71 (m, 2 H), 7.70-7.65 (m, 3 H), 7.59-7.55 (m, 1 H), 7.42 (t, J = 7.65 Hz, 1 H), 7.24- 7.20 (m, 2 H), 3.56 (t, J =5.65 Hz, 2 H), 3.48-3.44 (m, 4 H), 1.92 (s, 1.5 H), 1.86-1.80 (m, 2 H), 1.75- 1.68 (m, 2 H), 1.02 (t, J =7.53 Hz, 3 H). | | | | | | |
| 61 | 4-[6-Amino-8-(4′- fluoro-3′- methoxybiphenyl- 3-yl)-2,3,4,8- [1,5-a]pyrimidin- 8-yl]phenyl propane-1- sulfonate 0.75 acetate | 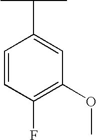 | CH₃CH₂CH₂SO₃ | A | 537 | |
| # 7.83-7.81 (m, 1 H), 7.70- 7.66 (m, 2 H), 7.58-7.55 (m, 1 H), 7.51-7.47 (m, 1 H), 7.36 (t, J = 7.78 Hz, 1 H), 7.32-7.24 (m, 2 H), 7.24-7.20 (m, 2 H), 7.08- 7.03 (m, 1 H), 3.91 (s, 3 H), 3.56 (t, J = 5.90 Hz, 2 H), 3.49-3.44 (m, 4 H), 1.91 (s, 2.1 H), 1.87-1.80 (m, 2 H), 1.75-1.68 (m, 2 H), 1.03 (t, J = 7.40 Hz, 3 H). | | | | | | |
| 62 | 4-[6-Amino-8-(3′- chloro-2′- fluorobiphenyl-3- yl)-2,3,4,8- tetrahydroimidazo [1,5-a]pyrimidin- 8-yl]phenyl propane-1- sulfonate 0.75 acetate | 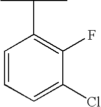 | CH₃CH₂CH₂SO₃ | A | 541 | |
| # 7.80-7.78 (m, 1 H), 7.70- 7.64 (m, 3 H), 7.62-7.57 (m, 1 H), 7.43-7.37 (m, 3 H), 7.33 (t, J = 7.65 Hz, 1 H), 7.25-7.20 (m, 2 H), 3.57-3.53 (m, 2 H), 3.47- 3.42 (m, 4 H), 1.92 (s, 2.2 H), 1.87-1.80 (m, 2 H), 1.74-1.67 (m, 2 H), 1.03 (t, J = 7.40 Hz, 3 H). | | | | | | |
| 63 | 4-[6-Amino-8- (2,5′- dichlorobiphenyl- 3-yl)-2,3,4,8- tetrahydroimidazo [1,5-a]pyrimidin- 8-yl]phenyl propane-1- sulfonate 0.75 acetate | 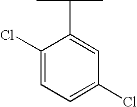 | CH₃CH₂CH₂SO₃ | A | 557/559 | |
| # 7.70 (s, 1 H), 7.67-7.63 (m, 2 H), 7.63-7.60 (m, 1 H), 7.59-7.56 (m, 1 H), 7.48-7.44 (m, 1 H), 7.41 (d, J = 2.51 Hz, 1 H), 7.37 (t, J = 7.65 Hz, 1 H), 7.28- 7.25 (m, 1 H), 7.22-7.19 (m, 2 H), 3.53 (t, J = 5.90 Hz, 2 H), 3.47-3.40 (m, 4 H), 1.90 (s, 2.3 H), 1.85- 1.79 (m, 2 H), 1.72- 1.65 (m, 2 H), 1.01 (t, J = 7.40 Hz, 3 H). | | | | | | |
| 64 | 4-{6-Amino-8-[3- (5- methoxypyridin-3- yl)phenyl]-2,3,4,8- tetrahydroimidazo [1,5-a]pyrimidin- 8-yl}phenyl propane-1- sulfonate 0.75 acetate | 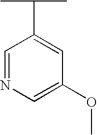 | CH₃CH₂CH₂SO₃ | A | 518 [M − 1]⁻ | |
| # 8.33 (d, J = 2.01 Hz, 1 H), 8.28 (d, J = 2.76 Hz, 1 H), 7.88 (t, J = 1.63 Hz, 1 H), 7.70-7.65 (m, 2 H), 7.64- 7.60 (m, 1 H), 7.57-7.53 (m, 1 H), 7.47-7.45 (m, 1 H), 7.40 (t, J = 7.65 Hz, 1 H), 7.23-7.19 (m, 2 H), 3.89 (s, 3 H), 3.57-3.53 (m, 2 H), 3.47-3.43 (m, 4 H), 1.90 (s, 2 H), 1.86- 1.77 (m, 2 H), 1.73-1.67 | | | | | | |
| # (m, 2 H), 1.01 (t, J = 7.40 Hz, 3 H). | | | | | | |
| 65 | 4-[6-Amino-8-(3′- methoxybiphenyl- 3-yl)-2,3,4,8- tetrahydroimidazo [1,5-a]pyrimidin- 8-yl]phenyl cyclopropane sulfonate 0.75 acetate | 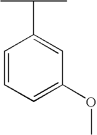 | cyclopropanSO₃ | A | 517 | |
| # 7.80-7.79 (m, 1 H), 7.69- 7.65 (m, 2 H), 7.55-7.52 (m, 1 H), 7.48-7.45 (m, 1 H), 7.39-7.32 (m, 2 H), 7.26-7.22 (m, 2 H), 7.10- 7.06 (m, 1 H), 7.04-7.02 (m, 1 H), 6.94-6.91 (m, 1 H), 3.80 (s, 3 H), 3.55 (t, J =6.02 Hz, 2 H), 3.44-3.40 (m, 2 H), 3.02-2.97 (m, 1 H), 1.90 (s, 2.5 H), 1.73- 1.66 (m, 2 H), 1.18-1.13 (m, 2 H), 1.03 -0.98 (m, 2 H), | | | | | | |
| 66 | 4-[6-Amino-8- (3,5′- dichlorobiphenyl- 3-yl)-2,3,4,8- tetrahydroimidazo [1,5-a]pyrimidin- 8-yl]phenyl cyclopropane- sulfonate 0.75 acetate | 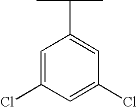 | cyclopropanSO₃ | A | 555/557 | |
| # 7.82 (t, J = 1.76 Hz, 1 H), 7.68-7.63 (m, 3 H), 7.60 (t, J = 1.88 Hz, 1 H), 7.56- 7.53 (m, 3 H), 7.39 (t, J =7.78 Hz, 1 H), 7.26-7.22 (m, 2 H), 3.55 (t, J = 5.90 Hz, 2 H), 3.45-3.39 (m, 4 H), 3.02-2.97 (m, 1 H), 1.90 (s, 2.4 H), 1.73-1.66 (m, 2 H), 1.18- 1.12 (m, 2 H), 1.03-0.98 (m, 2 H). | | | | | | |
| 67 | 4-[6-Amino-8-(3′- chlorobiphenyl-3- yl)-2,3,4,8- tetrahydroimidazo [1,5-a]pyrimidin- 8-yl]phenyl cyclopropane sulfonate 0.75 acetate | 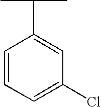 | cyclopropanSO₃ | A | 521 | |
| # 7.82 (t, J = 1.76 Hz, 1 H), 7.69-7.64 (m, 2 H), 7.63- 7.59 (m, 1 H), 7.56-7.53 (m, 1 H), 7.52-7.47 (m, 3 H), 7.44-7.40 (m, 1 H), 7.37 (t, J = 7.65 Hz, 1 H), 7.27-7.22 (m, 2 H), 3.55 (t, J = 5.90 Hz, 2 H), 3.43- 3.41 (m, 2 H) 3.02 -2.97 (m, 1 H), 1.89 (s, 2.5 H), 1.73-1.66 (m, 2 H), 1.18- 1.12 (m, 2 H), 1.03-0.98 (m, 2 H). | | | | | | |
| 68 | 4-[6-Amino-8-(3- pyridin-3- ylphenyl)-2,3,4,8- tetrahydroimidazo [1,5-a]pyrimidin- 8-yl]phenyl cyclopropane sulfonate 0.75 acetate | 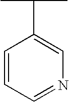 | cyclopropanSO₃ | A | 488 | |
| # 8.76-8.74 (m, 1 H), 8.58- 8.55 (m, 1 H), 7.94-7.91 (m, 1 H), 7.87-7.85 (m, 1 H), 7.70-7.66 (m, 2 H), 7.64-7.59 (m, 1 H), 7.55- 7.46 (m, 2 H), 7.40 (t, J =7.78 Hz, 1 H), 7.27-7.22 (m, 2 H), 3.55 (t, J = 5.52 Hz, 2 H), 3.45-3.42 (m, 2 H), 3.02-2.97 (m, 1 H), 1.90 (s, 2.5 H), 1.74-1.67 (m, 2 H), 1.18- 1.13 (m, 2 H), 1.04-0.99 (m, 2 H). | | | | | | |
| 69 | 4-{6-Amino-8-[3- (2-fluoropyridin- 3-yl)phenyl]- 2,3,4,8- tetrahydroimidazo [1,5-a]pyrimidin- 8-yl}phenyl cyclopropane sulfonate 0.75 acetate | 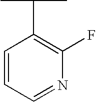 | cyclopropanSO₃ | A | 506 | |
| # 8.24-8.21 (m, 1 H), 8.02- 7.97 (m, 1 H), 7.81-7.79 (m, 1 H), 7.69-7.65 (m, 2 H), 7.63-7.60 (m, 1 H), 7.48-7.44 (m, 1 H), 7.44- 7.38 (m, 2 H), 7.26-7.22 (m, 2 H), 3.54 (t, J = 5.90 Hz, 2 H), 3.43-3.39 (m, 2 H), 3.02-2.97 (m, 1 H), 1.90 (s, 2 H), 1.72-1.66 (m, 2 H), 1.17-1.12 (m, 2 H), 1.03-0.99 (m, 2 H). | | | | | | |
| 70 | 4-{6-Amino-8-[3′- (trifluoroinethyl) biphenyl-3-yl]- 2,3,4,8- tetrahydroimidazo [1,5-a]pyrimidin- 8-yl}phenyl cyclopropane sulfonate 0.75 acetate | 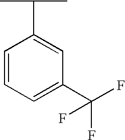 | cyclopropanSO₃ | A | 555 | |
| # 7.87-7.82 (m, 2 H), 7.79 (brs, 1 H), 7.74-7.70 (m, 2 H), 7.68-7.65 (m, 2 H), 7.64-7.61 (m, 1 H), 7.57- 7.54 (m, 1 H), 7.40 (t, J =7.78 Hz, 1 H), 7.26-7.23 (m, 2 H), 3.55 (t, J = 5.52 Hz, 2 H), 3.44-3.41 (m, 2 H), 3.02-2.97 (m, 1 H), 1.90 (s, 2.5 H), 1.73-1.67 (m, 2 H), 1.18-1.12 (m, 2 H), 1.03-0.98 (m, 2 H). | | | | | | |
| 71 | 4-[6-Amino-8-(3′- chloro-2′- fluorobiphenyl-3- yl)-2,3,4,8- tetrahydroimidazo [1,5-a]pyrimidin- 8-yl]phenyl cyclopropane sulfonate 0.75 acetate | 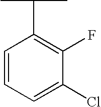 | cyclopropanSO₃ | A | 539/541 | |
| # 7.75-7.73 (m, 1 H), 7.68- 7.65 (m, 2 H), 7.64-7.56 (m, 2 H), 7.42-7.35 (m, 3 H), 7.31 (t, J = 7.65 Hz, 1 H), 7.26-7.23 (m, 2 H), 3.54 (t, J = 5.90 Hz, 2 H), 3.42-3.40 (m, 2 H), 3.01- 2.97 (m, 1 H), 1.90 (s, 2.6 H), 1.72-1.66 (m, 2 H), 1.17-1.12 (m, 2 H), 1.03- 0.98 (m, 2 H). | | | | | | |
| 72 | 4-[6-Amino-8- (2′,5′- dichlorobiphenyl- 3-yl)-2,3,4,8- tetrahydroimidazo [1,5-a]pyrimidin- 8-yl]phenyl cyclopropane sulfonate 0.5 acetate | 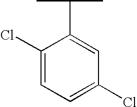 | cyclopropanSO₃ | A | 555/557 | |
| # 7.68-7.63 (m, 3 H), 7.62- 7.56 (m, 2 H), 7.48-7.44 (m, 1 H), 7.41-7.35 (m, 2 H), 7.29-7.22 (m, 3 H), 3.54 (t, J = 5.40 Hz, 2 H), 3.42-3.39 (m, 2 H), 3.01- 2.97 (m, 1 H), 1.90 (s, 1.3 H), 1.72-1.65 (m, 2 H), 1.18-1.12 (m, 2 H), 1.03- 0.98 (m, 2 H). | | | | | | |

### Examples 73-85

Examples 73-85 were synthesised as described for Method A (Example 31) in similar yields as exemplified in the Table below.

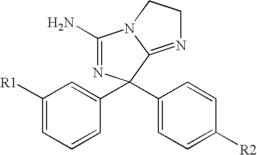

| | | | | | m/z | ¹H-NMR (DMSO-d₆) δ |
|---|---|---|---|---|---|---|
| Ex | Chemical name | R¹ | R² | Method | [M + 1]⁺ | ppm |
| 73 | 3′-[5-Amino-7-(4- methoxyphenyl)-2,7- dihydro-3 H- imiidazo[1,5- a]imidazol-7-yl]-5- methoxybiphenyl-3- yl methanesulfonate acetate | 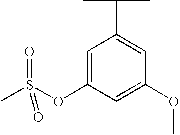 | OCH₃ | A | 507 | 7.84-7.81 (m, 1 H), 7.61- 7.57 (m, 1 H), 7.53-7.49 (m, 1 H), 7.49-7.45 (m, 2 H), 7.39 (t, J = 7.65 Hz, 1 H), 7.09-7.05 (m, 2 H), |
| # 6.95 (t, J = 2.26 Hz, 1 H), 6.86-6.82 (m, 2 H), 4.30- 4.24 (m, 2 H), 3.85 (s, 3 H), 3.69 (s, 3 H), 3.55-3.50 (m, 2 H), 3.42 (s, 3 H), 1.91 (s,3 H). | | | | | | |
| 74 | 4-[5-Amino-7-(3′- methoxybiphenyl-3- yl)-2,7-dihydro-3H- imidazo[1,5- a]imidazol-7- yl]phenyl methanesulfonate 0.25 acetate | 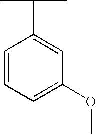 | CH₃SO₃ | A | 477 | 7.88-7.86 (m, 1 H), 7.73- 7.68 (m, 2 H), 7.60-7.56 (m, 1 H), 7.52-7.48 (m, 1 H), 7.41-7.36 (m, 2 H), 7.30-7.25 (m, 2 H) 7.11 (d, J = |
| # 8.28 Hz, 1 H), 7.07- 7.05 (m, 1 H), 6.96-6.92 (m, 1 H), 4.30 (t, J =8.66 Hz, 2 H), 3.80 (s, 3 H), 3.54, (t, J = 8.78 Hz, 2 H), 3.34 (s, 3 H), 1.91 (s, 1.4 H). | | | | | | |
| 75 | 4-[5-Amino-7-(3′,5′- dichlorobiphenyl-3- yl)-2,7-dihydro-3H- imidazo[1,5- a]imidazol-7- yl]phenyl methanesulfonate 0.25 acetate | 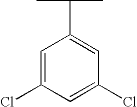 | CH₃SO₃ | A | 516 | 7.93-7.90 (m, 1 H), 7.72- 7.66 (m, 3 H), 7.62-7.56 (m, 4 H), 7.42 (t, J = 7.78 Hz, 1 H), 7.29-7.25 (m, 2 H), 4.30 (t, J = 8.78 Hz, 2 H), |
| # 3.53 (t, J = 8.91 Hz, 2 H), 3.34 (s, 3 H), 1.91 (s, 1.1 H). | | | | | | |
| 76 | 4-[5-Amino-7-(3′- chlorobiphenyl-3-yl)- 2,7-dihydro-3H- imidazo[1,5- a]imidazol-7- yl]phenyl methanesulfonate 0.5 acetate | 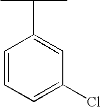 | CH₃SO₃ | A | 481 | 7.91-7.89 (m, 1 H), 7.73- 7.69 (m, 2 H), 7.66-7.62 (m, 1 H), 7.59-7.57 (m, 1 H), 7.54-7.48 (m, 3 H), 7.45-7.39 (m, 2 H), 7.29- 7.25 (m, 2 H), |
| # 4.30 (t, J =8.91 Hz, 2 H), 3.53 (t, J =8.66 Hz, 2 H), 3.34 (s, 3 H), 1.90 (s, 1.2 H). | | | | | | |
| 77 | 4-[5-Amino-7-(3- pyridin-3-ylphenyl)- 2,7-dihydro-3H- imidazo[1,5- a]imidazol-7- yl]phenyl methanesulfonate 0.5 acetate | 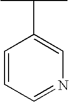 | CH₃SO₃ | A | 448 | 8.77 (d, J = 2.51 Hz, 1 H) 8.59-8.56 (m, 1 H), 7.97- 7.92 (m, 2 H), 7.74-7.70 (m, 2 H), 7.67-7.64 (m, 1 H), 7.58-7.54 (m, 1 H), 7.51-7.47 |
| # (m, 1 H), 7.44 (t, J = 7.65 Hz, 1 H), 7.29- 7.25 (m, 2 H), 4.31 (t, J =8.78 Hz, 2 H), 3.53 (t, J =8.78 Hz, 2 H), 3.34 (s, 3 H), 1.90 (s, 1.7 H). | | | | | | |
| 78 | 4-{5-Amino-7-[3-(2- fluoropyridin-3- yl)phenyl]-2,7- dihydro-3H- imidazo[1,5- a]imidazol-7- yl}phenyl methanesulfonate 0.5 acetate | 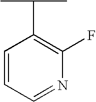 | CH₃SO₃ | A | 466 | 8.25-8.23 (m, 1 H), 8.05- 7.99 (m, 1 H), 7.88-7.86 (m, 1 H), 7.72-7.65 (m, 3 H), 7.49-7.43 (m, 3 H), 7.29-7.25 (m, 2 H), 4.29 (t, |
| # J = 8.78 Hz, 2 H), 3.52 (t, J = 8.91 Hz,2 H), 3.34 (s, 3 H), 1.83 (s, 1.3 H). | | | | | | |
| 79 | 4-{5-Amino-7-[3-(5- chloro-2- fluoropyridin-3- yl)phenyl]-2,7- dihydro-3 H- imidazo[1,5- a]imidazol-7- yl}phenyl methanesulfonate 0.5 acetate | 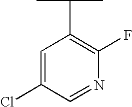 | CH₃SO₃ | A | 500 | 8.33-8.31 (m, 1 H), 8.21- 8.17 (m, 1 H), 7.90-7.87 (m, 1 H), 7.72-7.67 (m, 3 H), 7.54-7.45 (m, 2 H), 7.32-7.27 |
| # (m, 2 H), 4.30 (t, J = 8.66 Hz, 2 H), 3.60- 3.651 (m, 2 H), 3.35 (s, 3 H), 1.91 (s, 1.2 H). | | | | | | |
| 80 | 4-[5-Amino-7-(3′- methoxybiphenyl-3- yl)-2,7-dihydro-3H- imidazo[1,5- a]imidazol-7- yl]phenyl propane-2- sulfonate 0.5 acetate | 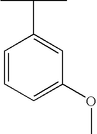 | isopropanSO₃ | A | 505 | 7.88-7.86 (m, 1 H), 7.72- 7.68 (m, 2 H), 7.60-7.57 (m, 1 H), 7.51-7.47 (m, 1 H), 7.41-7.36 (m, 2 H), 7.26-7.22 (m, 2 H), 7.12- 7.09 (m, |
| # 1 H), 7.07-7.05 (m, 1 H), 6.96-6.93 (m, 1 H), 4.30 (t, J = 8.91 Hz, 2 H), 3.80 (s, 3 H), 3.74- 3.64 (m, 1 H), 3.56-3.50 (m, 2 H), 1.90 (s, 1.3 H), 1.40 (s, 3 H), 1.38 (s, 3 H). | | | | | | |
| 81 | 4-[5-Amino-7-(3′,5′- dichlorobiphenyl-3- yl)-2,7-dihydro-3H- imidazo[1,5- a]imidazol-7- yl]phenyl propane-2- sulfonate 0.5 acetate | 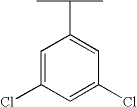 | isopropanSO₃ | A | 543/545 | 7.90 (t, J = 1.63 Hz, 1 H), 7.71-7.66 (m, 3 H), 7.62 (t, J = 1.88 Hz, 1 H), 7.59- 7.56 (m, 3 H), 7.42 (t, J =7.78 Hz, 1 H), 7.25-7.21 |
| # (m, 2 H), 4.30 (t, J = 8.78 Hz, 2 H), 3.71-3.64 (m, 1 H), 3.53 (t, J = 8.91 Hz, 2 H), 1.90 (s, 1.2 H), 1.40 (s, 3 H), 1.38 (s, 3 H). | | | | | | |
| 82 | 4-[5-Amino-7-(3′- chlorobiphenyl-3-yl)- 2,7-dihydro-3H- imidazo[1,5- a]imidazol-7- yl]phenyl propane-2- sulfonate 0.5 acetate | 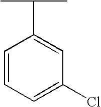 | isopropanSO₃ | A | 509/511 | 7.90 (t, J = 1.76 Hz, 1 H), 7.73-7.69 (m, 2 H), 7.67- 7.63 (m, 1 H), 7.61-7.58 (m, 1 H), 7.55-7.51 (m, 3 H), 7.46-7.40 (m, 2 H), 7.27-7.23 (m, |
| # 2 H), 4.32 (t, J = 8.66 Hz, 2 H), 3.72- 3.65 (m, 1 H), 3.55 (t, J =8.78 Hz, 2 H), 1.92 (s, 1.3 H), 1.42 (s, 3 H), 1.40 (s, 3 H). | | | | | | |
| 83 | 4-[5-Amino-7-(3- pyridin-3-ylphenyl)- 2,7-dihydro-3H- imidazo[1,5- a]imidazol-7- yl]phenyl propane-2- sulfonate 0.75 acetate | 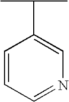 | isopropanSO₃ | A | 476 | 8.77 (d, J = 2.51 Hz, 1 H), 8.59-8.56 (m, 1 H), 7.97- 7.93 (m, 1 H), 7.92 (t, J =1.76 Hz, 1 H), 7.73-7.69 (m, 2 H), 7.66-7.63 (m, 1 H), |
| # 7.57-7.54 (m, 1 H), 7.51-7.47 (m, 1 H), 7.44 (t, J = 7.78 Hz, 1 H), 7.26- 7.22 (m, 2 H), 4.30 (t, J =8.78 Hz, 2 H), 3.71-3.64 (m, 1 H), 3.53 (t, J = 8.78 Hz, 2 H), 1.90 (s, 1.9 H), 1.40 (s, 3 H), 1.39 (s, 3 H). | | | | | | |
| 84 | 4-{5-Amino-7-[3-(2- fluoropyridin-3- yl)phenyl]-2,7- dihydro-3H- imidazo[1,5- a]imidazol-7- yl}phenyl propane-2- sulfonate 0.75 acetate | 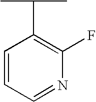 | isopropanSO₃ | A | 494 | 8.26-8.22 (m, 1 H), 8.05- 7.99 (m, 1 H), 7.85 (brs, 1 H), 7.71-7.64 (m, 3 H), 7.49-7.43 (m, 3 H), 7.26- 7.22 (m, 2 H), 4.29 (t, J =8.78 Hz, |
| # 2 H), 3.71-3.64 (m, 1 H), 3.52 (t, J = 8.91 Hz, 2 H), 1.90 (s, 2.5 H), 1.40 (s, 3 H), 1.39 (s, 3 H). | | | | | | |
| 85 | 4-{5-Amino-7-[3-(5- methoxypyridin-3- yl)phenyl]-2,7- dihydro-3H- imidazo[1,5- a]imidazol-7- yl}phenyl propane-2- sulfonate 0.5 acetate | 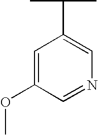 | isopropanSO₃ | A | 506 | 8.37-8.34 (m, 1 H), 8.31- 8.28 (m, 1 H), 7.91-7.89 (m, 1 H), 7.72-7.68 (m, 2 H), 7.65-7.61 (m, 1 H), 7.60-7.56 (m, 1 H), 7.49- 7.47 (m, 1 H), |
| # 7.44 (t, J =7.65 Hz, 1 H), 7.26-7.22 (m, 2 H),4.31 (t, J = 8.91 Hz, 2 H), 3.90 (s, 3 H), 3.71- 3.64 (m, 1 H), 3.55 (t, J =8.78 Hz, 2 H), 1.91 (s, 1.8 H), H), 1.40 (s, 3 H), 1.39 (s, 3 H). | | | | | | |

### Examples 86-87

Examples 86-87 were synthesised as described for Method A (Example 31) in similar yields as exemplified in the Table below.

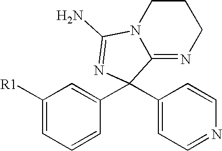

| | | | | [M + 1]⁺ | ¹H-NMR (DMSO-d₆) δ |
|---|---|---|---|---|---|
| Ex | Chemical name | R¹ | Method | m/z | ppm |
| 86 | 3′-(6-Amino-8-pyridin-4-yl- 2,3,4,8- tetrahydroimidazo[1,5- a]pyrimidin-8-yl)-5- chlorobiphenyl-3-yl methanesulfonate 0.5 acetate | 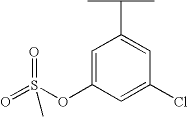 | A | 496 | 8.45 (dd, J = 4.52, 1.51 Hz, 2 H), 7.88 (s, 1 H), 7.81-7.66 (m, 1 H), 7.66-7.64 (m, 1 H), 7.61 (t, J = 1.63 Hz, 1 H), 7.57 (d, J = |
| # 8.28 Hz, 1 H), 7.54-7.50 (m, 2 H), 7.49-7.46 (m, 1 H), 7.41 (t, J = 7.78 Hz, 1 H), 3.54 (t, J =5.90 Hz, 2 H), 3.47 (s, 3 H), 3.45-3.39 (m, 2 H), 1.90 (s, 1.3 H), 1.73-1.66 (m, 2 H). | | | | | |
| 87 | 3′-(6-Amino-8-pyridin-4-yl- 2,3,4,8- tetrahydroimidazo[1,5- a]pyrimidin-8-yl)-5- methoxybiphenyl-3-yl methanesulfonate 0.25 acetate | 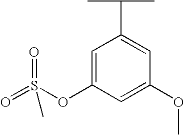 | A | 492 | 8.43 (s, 2 H), 7.87-7.83 (m, 1 H), 7.63-7.57 (m, 1 H), 7.52 (s, 2 H), 7.42-7.35 (m, 1 H), 7.07 (s, 1 H), 7.07-7.04 (m, 1 H), 6.98-6.91 (m, 1 H), 6.41- |
| # 6.29 (m, 1 H), 3.84 (s, 3 H), 3.54 (s, 2 H), 3.47-3.38 (m, 5 H), 1.94- 1.88 (m, 0.8 H), 1.70 (s, 2 H). | | | | | |

### Example 88

### 4-[5-Amino-7-(3-bromophenyl)-2,7-dihydro-3H-imidazo[1,5-a]imidazol-7-yl]phenol

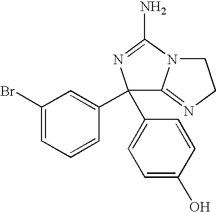

7-(3-Bromophenyl)-7-(4-hydroxyphenyl)-2,3,6,7-tetrahydro-5H-imidazo[1,5-a]imidazole-5-thione (0.6 g, 1.55 mmol) was dissolved in methanol (15 mL) and ammonium hydroxide (30%, 3 mL). tert-Butyl hydroperoxide (4.1 mL, 30 mmol, 70% in water) was added. The mixture was stirred over night and most of the methanol was evaporated, water and saturated aqueous sodium carbonate was added and the mixture was extracted with ethyl acetate. The organic extracts were pooled, washed with water, brine, dried over magnesium sulfate and evaporated, drying in vacuo oven afforded 0.4 g (71% yield) of the title product: ¹H NMR (DMSO-d₆) δ 10.73 (s, 2H), 7.70-7.68 (m, 1H), 7.55-7.52 (m, 1H), 7.38-7.34 (m, 1H), 7.31-7.27 (m, 2H), 7.23 (t, J=7.91 Hz, 1H), 6.68-6.64 (m, 2H), 4.28-4.22 (m, 2H), 3.53-3.46 (m, 2H).

### Example 89

### 4-[5-Amino-7-(3′-chlorobiphenyl-3-yl)-2,7-dihydro-3H-imidazo[1,5-a]imidazol-7-yl]phenol

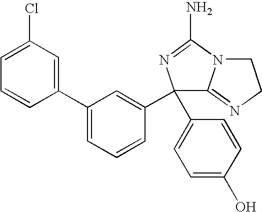

4-[5-Amino-7-(3-bromophenyl)-2,7-dihydro-3H-imidazo[1,5-a]imidazol-7-yl]phenol (0.27 g, 0.73 mmol), cesium carbonate (0.71 g, 2.2 mmol), 3-chlorobenzeneboronic acid (0.16 g, 1.02 mmol) and [1,1′-bis(diphenylphosphino)ferrocene]palladium(II) chloride dichloromethane adduct (30 mg, 0.04 mmol) was dissolved in dimethoxyethane:ethanol:water (6:3:1) and heated to 130° C. for 20 min in a microwave. The mixture was filtered through celite, diluted with ethyl acetate and washed with water and brine, dried over magnesium sulfate and concentrated. Column chromatography, gradient elution with 0-10% ammonia (7 N in methanol) in dichloromethane as solvent gave 0.115 g (39% yield) of the title compound: ¹H NMR (DMSO-d₆) δ 7.85-7.82 (m, 1H), 7.61-7.55 (m, 2H), 7.52-7.47 (m, 3H), 7.45-7.41 (m, 1H), 7.40-7.33 (m, 3H), 6.68-6.63 (m, 2H), 4.29-4.22 (m, 2H), 3.53-3.46 (m, 2H); MS (ES) m/z 401 [M−1]⁻.

### Example 90

### 4-[5-Amino-7-(3′-chlorobiphenyl-3-yl)-2,7-dihydro-3H-imidazo[1,5-a]imidazol-7-yl]phenyl trifluoromethanesulfonate 0.75 acetate

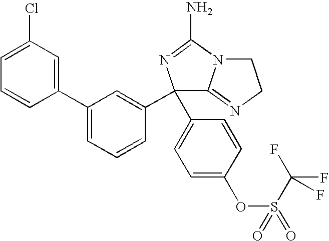

4-[5-Amino-7-(3′-chlorobiphenyl-3-yl)-2,7-dihydro-3H-imidazo[1,5-a]imidazol-7-yl]phenol (115 mg, 0.285 mmol), 1,1,1-trifluoro-N-phenyl-N-[(trifluoromethyl)sulfonyl]methanesulfonamide (98 mg, 0.3 mmol) and potassium carbonate (0.24 g, 1.7 mmol) was dissolved in dry tetrahydrofuran (5 mL) and heated to 120° C. for 12 min in the microwave. After cooling ethyl acetate and water was added. The organic phase was filtered and dimethyl sulfoxide (2 mL) was added. The solution was concentrated in vacuo to remove ethyl acetate and purified by preparative HPLC to give 36 mg (21% yield) of the title compound: ¹H NMR (DMSO-d₆) δ 7.89-7.86 (m, 1H), 7.81-7.77 (m, 2H), 7.64-7.61 (m, 1H), 7.58-7.57 (m, 1H), 7.55-7.49 (m, 3H), 7.48-7.40 (m, 4H), 4.31 (t, J=8.78 Hz, 2H), 3.54 (t, J=8.91 Hz, 2H), 1.90 (s, 2H); MS (ES) m/z 533 [M−1]⁻.

### Example 91

### 4-[6-Amino-8-(3-bromophenyl)-2,3,4,8-tetrahydroimidazo[1,5-a]pyrimidin-8-yl]phenol

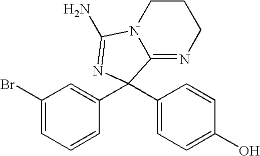

The title compound was prepared as described in example 88 starting with 8-(3-bromophenyl)-8-(4-hydroxyphenyl)-3,4,7,8-tetrahydroimidazo[1,5-a]pyrimidine-6(2H)-thione in 98% yield: ¹H NMR (DMSO-d₆) δ 10.73 (s, 2H), 7.65 (t, J=1.88 Hz, 1H), 7.54-7.50 (m, 1H), 7.36-7.32 (m, 1H), 7.28-7.24 (m, 2H), 7.20 (t, J=7.91 Hz, 1H), 6.65-6.61 (m, 2H), 3.54-3.49 (m, 2H), 3.40-3.36 (m, 2H), 1.70-1.64 (m, 2H).

### Example 92

### 4-[6-Amino-8-(3′-chlorobiphenyl-3-yl)-2,3,4,8-tetrahydroimidazo[1,5-a]pyrimidin-8-yl]phenol

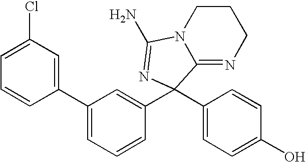

The title compound was prepared as described in example 89 starting with 4-[6-amino-8-(3-bromophenyl)-2,3,4,8-tetrahydroimidazo[1,5-a]pyrimidin-8-yl]phenol in 18% yield: ¹H NMR (DMSO-d₆) δ 7.81-7.76 (m, 1H), 7.59-7.53 (m, 2H), 7.50-7.45 (m, 3H), 7.43-7.39 (m, 1H), 7.36-7.27 (m, 3H), 6.65-6.60 (m, 2H), 3.54-3.50 (m, 2H), 3.41-3.36 (m, 2H), 1.71-1.64 (m, 2H); MS (ES) m/z 415 [M−1]⁻.

### Example 93

### 4-[6-Amino-8-(3′-chlorobiphenyl-3-yl)-2,3,4,8-tetrahydroimidazo[1,5-a]pyrimidin-8-yl]phenyl trifluoromethanesulfonate acetate

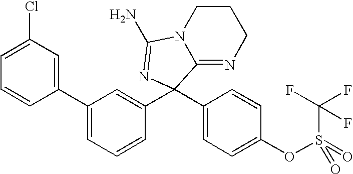

The title compound was prepared as described in example 90 starting with 4-[6-amino-8-(3′-chlorobiphenyl-3-yl)-2,3,4,8-tetrahydroimidazo[1,5-a]pyrimidin-8-yl]phenol in 28% yield: ¹H NMR (DMSO-d₆) δ 7.82 (t, J=1.76 Hz, 1H), 7.78-7.74 (m, 2H), 7.63-7.59 (m, 1H), 7.56-7.53 (m, 1H), 7.52-7.48 (m, 3H), 7.44-7.36 (m, 4H), 3.55 (t, J=5.77 Hz, 2H), 3.44-3.42 (m, 2H), 1.89 (s, 3H), 1.72-1.67 (m, 2H); MS (ES) m/z 547 [M−1]⁻.

### Example 94

### 1-(3-Bromophenyl)-1-phenylmethanamine

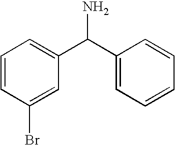

3-Bromobenzonitrile (10.92 g, 60 mmol) was added to a solution of bromo(phenyl)magnesium (24 mL, 72 mmol) in dry tetrahydrofuran (25 mL) at ambient temperature under an atmosphere of argon. The resulting mixture was stirred at 60° C. for 4 h, then cooled to 0° C. and dry methanol (60 mL) was added. Sodium borohydride (5.68 g, 150 mmol) was added in three portions at 0° C. under an atmosphere of argon and the resulting mixture was allowed to reach ambient temperature and stirred for 1.5 h. The reaction was quenched by addition of a saturated aqueous solution of ammonium chloride. The mixture was diluted with dichloromethane and the organic phase separated. The aqueous phase was extracted with dichloromethane and the combined organic phases were concentrated to give 17.1 g (quantative yield) of the title compound: MS (EI) m/z 261, 263 [M+1]⁺.

### Example 95

### 1-Bromo-3-[isothiocyanato(phenyl)methyl]benzene

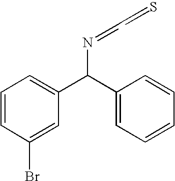

The title compound was prepared as described for example 9 in quantitative yield starting from 1-(3-bromophenyl)-1-phenylmethanamine: MS (ESI) m/z 302, 304 [M−1]⁻.

### Example 96

### 4-(3-Bromophenyl)-4-phenyl-1,3-thiazolidine-2,5-dithione

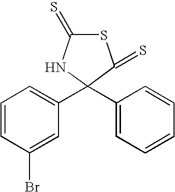

The title compound was prepared as described for example 11 in quantitative yield starting from 1-bromo-3-[isothiocyanato(phenyl)methyl]benzene: MS (ES) m/z 380, 382 [M+1]⁺.

### Example 97

### 8-(3-Bromophenyl)-8-phenyl-3,4,7,8-tetrahydroimidazo[1,5-a]pyrimidine-6(2H)-thione

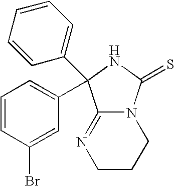

The title compound prepared as described for example 13 in 90% yield starting from 4-(3-bromophenyl)-4-phenyl-1,3-thiazolidine-2,5-dithione: MS (ES) m/z 386, 388 [M+1]⁺.

### Example 98

### 8-(3-Bromophenyl)-8-phenyl-2,3,4,8-tetrahydroimidazo[1,5-a]pyrimidin-6-amine

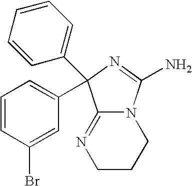

The title compound was prepared as described for example 23 in 19% yield starting from 8-(3-bromophenyl)-8-phenyl-3,4,7,8-tetrahydroimidazo[1,5-a]pyrimidine-6(2H)-thione: MS (ES) m/z 369, 371 [M+1]⁺.

### Example 99

### 3′-(6-Amino-8-phenyl-2,3,4,8-tetrahydroimidazo[1,5-a]pyrimidin-8-yl)-5-methoxybiphenyl-3-yl methanesulfonate hydrochloride

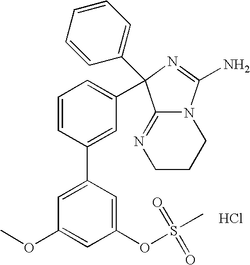

8-(3-Bromophenyl)-8-phenyl-2,3,4,8-tetrahydroimidazo[1,5-a]pyrimidin-6-amine (81 mg, 0.22 mmol), potassium carbonate (0.18 g, 1.32 mmol), 3-methoxy-5-(4,4,5,5-tetramethyl-1,3,2-dioxaborolan-2-yl)phenyl methanesulfonate (100 mg, 0.31 mmol) and [1,1′-bis(diphenylphosphino)ferrocene]palladium(II) chloride dichloromethane adduct (18 mg, 0.02 mmol) was dissolved in tetrahydrofuran and heated to 130° C. for 4 h in a microwave. The reaction mixture was diluted with water and extracted with diethyl ether. The organic layer was concentrated in vacuo and the crude was purified by preparative HPLC. The residue was diluted with 1 M sodium hydroxide and extracted with dichloromethane. Hydrochloric acid (1 M in diethyl ether, 0.5 mL) was added and the solvent was evaporated to give 28 mg (24% yield) of the title compound: ¹H NMR (DMSO-d₆) δ 9.13 (br s, 2H), 7.76-7.70 (m, 2H), 7.55-7.47 (m, 2H), 7.43-7.36 (m, 5H), 7.23-7.17 (m, 2H), 7.00-6.94 (m, 1H), 3.86 (s, 3H), 3.83-3.77 (m, 2H), 3.43 (s, 3H), 1.91-1.83 (m, 2H); MS (ES) m/z 491 [M+1]⁺.

### Example 100

### 1-(3-Bromophenyl)-1-(3-methoxyphenyl)methanamine

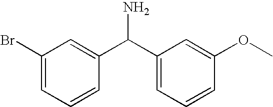

The title compound was prepared in 89% yield as described in example 7 starting with 3-bromoanisole: ¹H NMR (DMSO-d₆) δ 7.63 (m, 1H), 7.38 (m, 2H), 7.28-7.16 (m, 2H), 7.01 (m, 1H), 6.94 (m, 1H), 6.76 (m, 1H), 5.05 (s, 1H), 3.73 (s, 3H), 2.33 (br s, 2H); MS (ES) m/z 293 [M+1]⁺.

### Example 101

### 1-Bromo-3-[isothiocyanato(3-methoxyphenyl)methyl]benzene

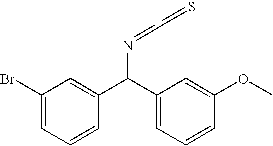

The title compound was prepared in 93% yield as described in example 9 starting with 1-(3-bromophenyl)-1-(3-methoxyphenyl)methanamine: ¹H NMR (CDCl₃) δ 7.46-7.42 (m, 2H), 7.32-7.19 (m, 3H), 6.90-6.81 (m, 3H), 5.91 (s, 1H), 3.80 (s, 3H).

### Example 102

### 4-(3-Bromophenyl)-4-(3-methoxyphenyl)-1,3-thiazolidine-2,5-dithione

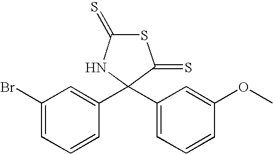

The title compound was prepared in quantitative yield as described in example 11 starting with 1-bromo-3-[isothiocyanato(3-methoxyphenyl)methyl]benzene: MS (ES) m/z 411 [M+1]⁺.

### Example 103

### 8-(3-Bromophenyl)-8-(3-methoxyphenyl)-3,4,7,8-tetrahydroimidazo[1,5-a]pyrimidine-6(2H)-thione

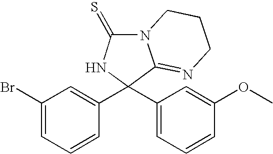

The title compound was prepared in 68% yield as described in example 13 starting with 4-(3-bromophenyl)-4-(3-methoxyphenyl)-1,3-thiazolidine-2,5-dithione: MS (ES) m/z 417 [M+1]⁺.

### Example 104

### 8-(3-Bromophenyl)-8-(3-hydroxyphenyl)-3,4,7,8-tetrahydroimidazo[1,5-a]pyrimidine-6(2H)-thione

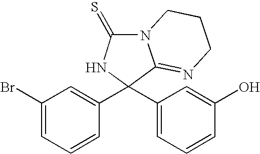

The title compound was prepared in quantitative yield as described in example 14 starting with 8-(3-bromophenyl)-8-(3-methoxyphenyl)-3,4,7,8-tetrahydroimidazo[1,5-a]pyrimidine-6(2H)-thione: MS (ES) m/z 403 [M+1]⁺.

### Example 105

### 3-[8-(3-Bromophenyl)-6-thioxo-2,3,4,6,7,8-hexahydroimidazo[1,5-a]pyrimidin-8-yl]phenyl methanesulfonate

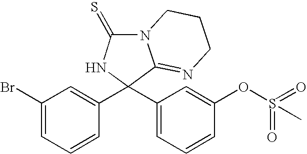

The title compound was prepared in 59% yield as described in example 15 starting with 8-(3-bromophenyl)-8-(3-hydroxyphenyl)-3,4,7,8-tetrahydroimidazo[1,5-a]pyrimidine-6(2H)-thione and methanesulfonyl chloride: MS (ES) m/z 481 [M+1]⁺.

### Example 106

### 3-[8-(3-Bromophenyl)-6-thioxo-2,3,4,6,7,8-hexahydroimidazo[1,5-a]pyrimidin-8-yl]phenyl propane-1-sulfonate

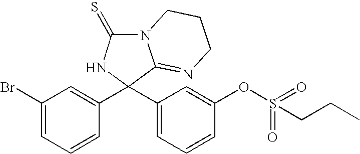

The title compound was prepared in 34% yield as described in example 15 starting with 8-(3-bromophenyl)-8-(3-hydroxyphenyl)-3,4,7,8-tetrahydroimidazo[1,5-a]pyrimidine-6(2H)-thione and 1-propanesulfonylchloride: MS (ES) m/z 509 [M+1]⁺.

### Example 107

### 3-[8-(3-Bromophenyl)-6-thioxo-2,3,4,6,7,8-hexahydroimidazo[1,5-a]pyrimidin-8-yl]phenyl cyclopropanesulfonate

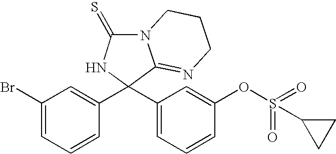

The title compound was prepared in 38% yield as described in example 15 starting with 8-(3-bromophenyl)-8-(3-hydroxyphenyl)-3,4,7,8-tetrahydroimidazo[1,5-a]pyrimidine-6(2H)-thione and cyclopropanesulfonyl chloride: MS (ES) m/z 507 [M+1]⁺.

### Example 108

### 8-(3-Bromophenyl)-8-(3-methoxyphenyl)-2,3,48-tetrahydroimidazo[1,5-a]pyrimidin-6-amine

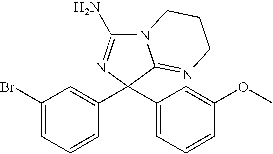

The title compound was prepared in quantitative yield as described in example 23 starting with 8-(3-bromophenyl)-8-(3-methoxyphenyl)-3,4,7,8-tetrahydroimidazo[1,5-a]pyrimidine-6(2H)-thione: MS (ES) m/z 400 [M+1]⁺.

### Example 109

### 3-[6-Amino-8-(3-bromophenyl)-2,3,4,8-tetrahydroimidazo[1,5-a]pyrimidin-8-yl]phenol

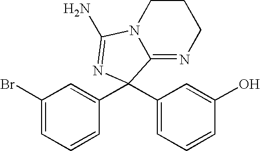

The title compound was prepared in quantitative yield as described in example 14 starting with 8-(3-bromophenyl)-8-(3-methoxyphenyl)-2,3,4,8-tetrahydroimidazo[1,5-a]pyrimidin-6-amine: MS (ES) m/z 386 [M+1]⁺.

### Example 110

### 3-[6-Amino-8-(3-bromophenyl)-2,3,4,8-tetrahydroimidazo[1,5-a]pyrimidin-8-yl]phenyl methanesulfonate

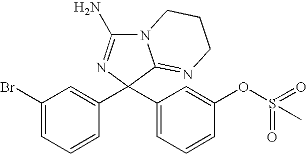

The title compound was prepared in quantitative yield as described in example 23 starting with 3-[8-(3-bromophenyl)-6-thioxo-2,3,4,6,7,8-hexahydroimidazo[1,5-a]pyrimidin-8-yl]phenyl methanesulfonate: MS (ES) m/z 464 [M+1]⁺.

### Example 111

### 3-[6-Amino-8-(3-bromophenyl)-2,3,4,8-tetrahydroimidazo[1,5-a]pyrimidin-8-yl]phenyl propane-1-sulfonate

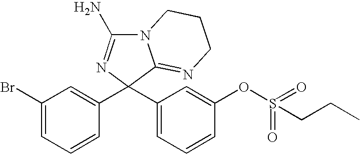

The title compound was prepared in 81% yield as described in example 23 starting with 3-[8-(3-Bromophenyl)-6-thioxo-2,3,4,6,7,8-hexahydroimidazo[1,5-a]pyrimidin-8-yl]phenyl propane-1-sulfonate: MS (ES) m/z 492 [M+1]⁺.

### Example 112

### 3-[6-Amino-8-(3-bromophenyl)-2,3,4,8-tetrahydroimidazo[1,5-a]pyrimidin-8-yl]phenyl cyclopropanesulfonate

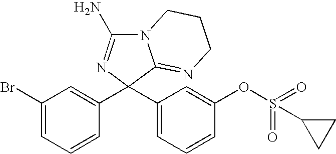

The title compound was prepared in 80% yield as described in example 23 starting with 3-[8-(3-bromophenyl)-6-thioxo-2,3,4,6,7,8-hexahydroimidazo[1,5-a]pyrimidin-8-yl]phenyl cyclopropanesulfonate: MS (ES) m/z 490 [M+1]⁺.

### Example 113

### 3-[6-Amino-8-(3-bromophenyl)-2,3,4,8-tetrahydroimidazo[1,5-a]pyrimidin-8-yl]phenyl trifluoromethanesulfonate

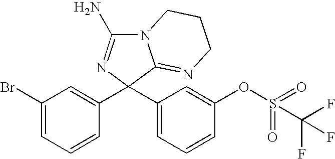

3-[6-Amino-8-(3-bromophenyl)-2,3,4,8-tetrahydroimidazo[1,5-a]pyrimidin-8-yl]phenol (0.83 g, 2.1 mmol) was mixed with 1,1,1-trifluoro-N-phenyl-N-[(trifluoromethyl)sulfonyl]methanesulfonamide (0.77 g, 2.1 mmol) in dichloromethane at 0° C. Triethylamine (0.30 mL, 2.1 mmol) was added and the mixture was stirred at 25° C. for 12 h. Aqueous potassium carbonate and ethyl acetate was added and the organic phase was collected, dried over sodium sulfate and evaporation of the solvent in vacuo gave 1.5 g (138% yield) of the title compound: MS (ES) m/z 518 [M+1]⁺.

Method C:

### Example 114

### 3-{6-Amino-8-[3′,5′-bis(trifluoromethyl)biphenyl-3-yl]-2,3,4,8-tetrahydroimidazo[1,5-a]pyrimidin-8-yl}phenyl methanesulfonate

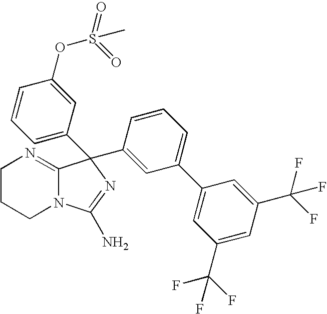

3-[6-Amino-8-(3-bromophenyl)-2,3,4,8-tetrahydroimidazo[1,5-a]pyrimidin-8-yl]phenyl methanesulfonate (93 mg, 0.20 mmol) was dissolved in dry dioxane (3 mL), and potassium carbonate (150 mg, 1.1 mmol) was added. Nitrogen was bubbled through the solution for 5 min, [1,1′-bis(diphenylphosphino)ferrocene]palladium(II) chloride dichloromethane adduct (30 mg, 0.04 mmol) was added and the vial sealed. The reaction was heated at 100° C. for 12 h. Ethyl acetate and water was added and the aqueous phase was dried over sodium sulfate. Evaporation of the solvent in vacuo was followed by purification by preparative HPLC to give (6 mg, 5%) the title product: ¹H NMR (MeOH-d₄) δ 8.17 (m, 2H), 7.94 (m, 1H), 7.71 (m, 1H), 7.66 (m, 1H), 7.59-7.53 (m, 2H), 7.48 (m, 1H), 7.43-7.26 (m, 3H), 3.76 (m, 2H), 3.57 (m, 2H), 3.20 (s, 3H), 1.96-1.88 (m, 2H); MS (ES) m/z 597 [M+1]⁺.

Method D:

### Example 115

### 3-[6-Amino-8-(3′-chlorobiphenyl-3-yl)-2,3,4,8-tetrahydroimidazo[1,5-a]pyrimidin-8-yl]phenyl trifluoromethanesulfonate

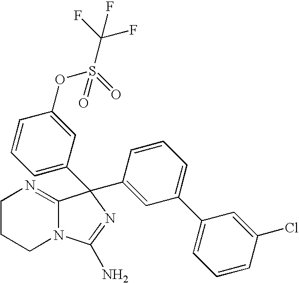

3-[6-Amino-8-(3-bromophenyl)-2,3,4,8-tetrahydroimidazo[1,5-a]pyrimidin-8-yl]phenyl trifluoromethanesulfonate (103 mg, 0.20 mmol) was dissolved in dry dioxane (3 mL) and (3-chlorophenyl)boronic acid (63 mg, 0.40 mmol) and potassium carbonate (150 mg, 1.1 mmol) was added. Nitrogen was bubbled through the solution for 5 min, [1,1′-bis(diphenylphosphino)ferrocene]palladium(II) chloride dichloromethane adduct (30 mg, 0.04 mmol) was added and the vial sealed. The reaction was started at room temperature and then warmed slowly up to 100° C. where the heating was continued for 2 h. Ethyl acetate and water was added and the aqueous phase was dried over sodium sulfate. Evaporation of the solvent in vacuo was followed by purification by preparative HPLC to give (3 mg, 3%) the title product: ¹H NMR (MeOH-d₄) δ 7.60 (m, 1H), 7.58-7.52 (m, 4 H), 7.51-7.45 (m, 3H), 7.42-7.32 (m, 4H), 3.76 (m, 2H), 3.56 (m, 2H), 1.92 (m, 2H). MS (ES) m/z 549 [M+1]⁺.

### Examples 116-133

Examples 116-133 were synthesised as described in example 114 (method C) or example 115 (method D) in similar yields as seen in the table below.

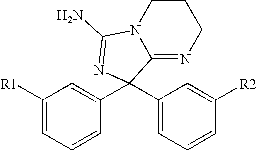

| | | | | | | | | ¹H-NMR |
|---|---|---|---|---|---|---|---|---|
| | | | | | Reaction | | [M + 1 ⁺ | (MeOH- |
| Ex | Chemical name | R1 | R2 | Method | Time | | m/z | (MeOH-d₄) δ ppm |
| 116 | 3-[6-Amino-8-(3′,5′- dichlorobiphenyl-3-yl)- 2,3,4,8- tetrahydroimidazo[1,5- a]pyrimidin-8-yl]phenyl methanesulfonate | 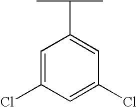 | CH₃SO₃ | C | 12 | h | 529 | |
| 117 | 3-[6-Amino-8-(3′- chlorobiphenyl-3-yl) 2,3,4,8- tetrahydroimidazo[1,5 a]pyrimidin-8-yl]phenyl methanesulfonate | 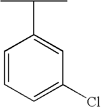 | CH₃SO₃ | C | 2 | days | 495 | |
| 118 | 3-[6-Amino-8-(3′- methoxybiphenyl-3-yl)- 2,3,4,8- tetrahydroimidazo[1,5- a]pyrimidin-8-yl]phenyl methanesulfonate | 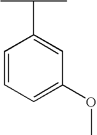 | CH₃SO₃ | C | 4 | days | 491 | |
| 119 | 3-[6-Amino-8-(3-pyridin- 3-ylphenyl)-2,3,4,8- tetrahydroimidazo[1,5- a]pyrimidin-8-yl]phenyl methanesulfonate | 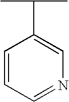 | CH₃SO₃ | C | 4 | days | 462 | |
| 120 | 3-[6-Amino-8-(3- pyrimidin-5-ylphenyl)- 2,3,4,8- tetrahydroimidazo[1,5 a]pyrimidin-8-yl]phenyl methanesulfonate | 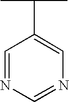 | CH₃SO₃ | C | 4 | days | 463 | |
| 121 | 3-{6-Amino-8-[3-(5- chloro-2-fluoropyridin-3- yl)phenyl]-2,3,4,8- tetrahydroimidazo[1,5- a]pyrimidin-8-yl}phenyl methanesulfonate | 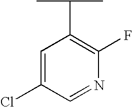 | CH₃SO₃ | C | 2 | days | 514 | |
| 122 | 3-[6-Amino-8-(3′, 5′- dimethylbiphenyl- 3-yl)- 2,3,4,8- tetrahydroimidazo [1,5- a]pyrimidin-8-yl}phenyl methanesulfonate | 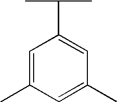 | CH₃SO₃ | C | 12 | h | 489 | 7.59-7.54 (m, 2 H), 7.52- 7.41 (m, 4 H), 7.39-7.31 (m, 2 H), 7.16 (m, 2 H), |
| # 6.98 (m, 1 H), 3.78 (m, 2 H), 3.58 (m, 2 H), 3.20 (s, 3 H), 2.33 (s, 6 H), 1.96- 1.90 (m, 2 H). | | | | | | | | |
| 123 | 3-[6-Amino-8-(3′,5′- dichlorobiphenyl-3-yl)- 2,3,4,8- tetrahydroimidazo[1,5- a]pyrimidin-8-yl]phenyl propane-1-sulfonate | 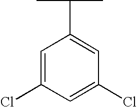 | CH₃CH₂CH₂SO₃ | C | 12 | h | 557 | |
| 124 | 3-[6-Amino-8-(3′- methoxybiphenyl-3-yl)- 2,3,4,8- tetrahydroimidazo[1,5- a]pyrimidin-8-yl]phenyl propane-1-sulfonate | 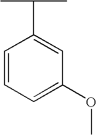 | CH₃CH₂CH₂SO₃ | C | 12 | h | 519 | |
| 125 | 3-[6-Amino-8-(3- pyrimidin-5-ylphenyl)- 2,3,4,8- tetrahydroimidazo[1,5- a]pyrimidin-8-yl]phenyl propane-1-sulfonate | 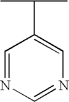 | CH₃CH₂CH₂SO₃ | C | 12 | h | 491 | |
| 126 | 3-[6-Amino-8-(3′,5′- dichlorobiphenyl-3-yl)- 2,3,4,8- tetrahydroimidazo[1,5- a]pyrimidin-8-yl]phenyl cyclopropanesulfonate | 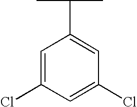 | cyclopropanSO₃ | C | 12 | h | 555 | |
| 127 | 3-[6-Amino-8-(3′- methoxybiphenyl-3-yl)- 2,3,4,8- tetrahydroimidazo[1,5- a]pyrimidin-8-yl]phenyl cyclopropanesulfonate | 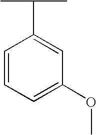 | cyclopropanSO₃ | C | 12 | h | 517 | |
| 128 | 3-[6-Amino-8-(3′, 5′- dichlorobiphenyl- 3-yl)- 2,3,4,8- tetrahydroimidazo [1,5- a]pyrimidin-8-yl]phenyl trifluoro methanesulfonate | 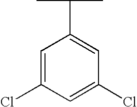 | CF₃SO₃ | D | 2 | h | 583 | |
| 129 | 3-[6-Amino-8-(3′- methoxybiphenyl-3-yl)- 2,3,4,8- tetrahydroimidazo[1,5- a]pyrimidin-8-yl]phenyl trifluoromethanesulfonate | 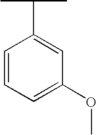 | CF₃SO₃ | D | 2 | h | 545 | |
| 130 | 3-[6-Amino-8-(3-pyridin- 3-ylphenyl)-2,3,4,8- tetrahydroimidazo[1,5- a]pyrimidin-8-yl]phenyl trifluoromethanesulfonate | 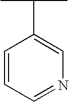 | CF₃SO₃ | D | 2 | h | 516 | |
| 131 | 3-{6-Amino-8-[3-(5- chloro-2-fluoropyridin-3- yl)phenyl]-2,3,4,8- tetrahydroimidazo[1,5- a]pyrimidin-8-yl}phenyl trifluoromethanesulfonate | 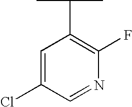 | CF₃SO₃ | D | 2 | h | 568 | |
| 132 | 3-[6-Amino-8-(3- pyrimidin-5-ylphenyl- 2,3,4,8- tetrahydroimidazo[1,5- a]pyrimidin-8-yl}phenyl trifluoromethanesulfonate | 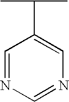 | CF₃SO₃ | D | 2 | h | 517 | 9.13 (s, 1 H), 9.01 (s, 2 H), 7.70 (m, 1 H), 7.62 (m, 1 H), 7.59-7.48 (m, 4 H), 7.43 (m, 1 H), 7.35 (m, 1 H), |
| # 3.75 (m, 2 H), 3.54 (m, 2 H), 1.91 (m, 2 H). | | | | | | | | |
| 133 | 3′-[6-Amino-8-(3- methoxyphenyl)-2,3,4,8- tetrahydroimidazo[1,5- a]pyrimidin-8-yl]-5- methoxybiphenyl-3-yl methanesulfonate | 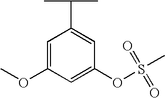 | CH₃O | C | 4 | days | 521 | |

### Example 134

### N-tert-Butanesulfinyl 3-bromophenyl-aldimine

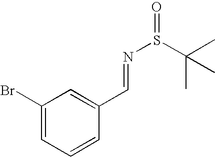

A mixture of 3-bromo-benzaldehyde (3.7 g, 20 mmol), N-tert-butanesulfinamide (2.4 g, 20 mmol) and titanium tetraethoxide (9.1 g, 40 mmol) in tetrahydrofuran (10 mL) was heated at 65° C. for 12 h. Evaporation of solvent onto silica gel and purification by chromatography using an eluent gradient of ethyl acetate in heptane (0-100%) gave 4.9 g (84%) of the title compound: MS m/z (ES) 290 [M+1]⁺.

### Example 135

### 3-[Amino(3-bromophenyl)methyl]-N,N-dimethylbenzamide

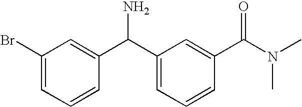

3-Iodo-N,N-dimethylbenzamide (1.0 g, 3.6 mmol) was dissolved in toluene (40 mL) and isopropyl magnesium chloride (1M in tetrahydrofuran, 4 mL, 4 mmol) was added at −40° C. The reaction was stirred at 40° C. for 1 h, then N-tert-butanesulfinyl 3-bromophenyl-aldimine (1.0 g, 3.6 mmol) in toluene (2 mL) was added and the reaction was allowed to warm to −10° C. and kept at that temperature for 3 h. The reaction was quenched by the addition of aqueous ammonium chloride. Aqueous workup and extraction with ethyl acetate, followed by purification by chromatography on silica using an eluent with methanol in dichloromethane (0-5%) gave the intermediate sulfinamide (1.0 g, 2.2 mmol). The intermediate was treated with hydrochloric acid (1M in diethyl ether, 3 equivalents) in methanol/diethyl ether (5 mL) for 30 minutes followed by concentration in vacuo. The crude was partitioned between ethyl acetate and aqueous potassium carbonate, dried over potassium carbonate and concentrated in vacuo to give 1.0 g (63%) of the title compound: MS m/z (APCI) 335 [M+1]⁺.

### Example 136

### 4-[Amino(3-bromophenyl)methyl]-N,N-dimethylbenzamide

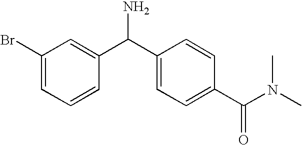

The title compound was synthesized in 36% yield as described in example 135 starting from 4-iodo-N,N-dimethylbenzamide: MS m/z (APCI) 335 [M+1]⁺.

### Example 137

### 3-[(3-Bromophenyl)(isothiocyanato)methyl]-N,N-dimethylbenzamide

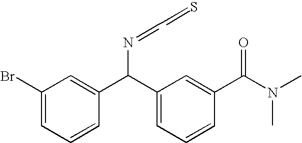

Thiocarbonyldiimidazole (0.39 g, 2.2 mmol) was added in portions to a stirred solution of 3-[amino(3-bromophenyl)methyl]-N,N-dimethylbenzamide (1.0 g, 2.2 mmol) in dichloromethane (20 mL) at 25° C. After stirring for 2 h the solution was washed with brine, dried over sodium sulfate and evaporated, to give 0.83 g of the title compound in quantitative yield: MS m/z (APCI) 377 [M+1]⁺.

### Example 138

### 4-[(3-Bromophenyl)(isothiocyanato)methyl]-N,N-dimethylbenzamide

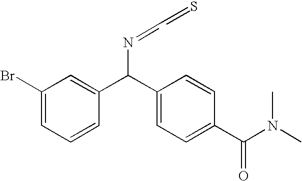

The title compound was synthesized in quantitative yield (0.056 g) as described in example 137 starting from 4-[amino(3-bromophenyl)methyl]-N,N-dimethylbenzamide: MS m/z (APCI) 377 [M+1]⁺.

### Example 139

### 3-[4-(3-Bromophenyl)-2,5-dithioxo-1,3-thiazolidin-4-yl]-N,N-dimethylbenzamide

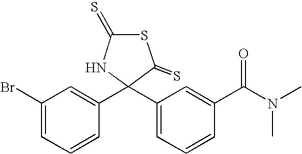

A solution of 3-[(3-bromophenyl)(isothiocyanato)methyl]-N,N-dimethylbenzamide (0.83 g, 2.2 mmol) and carbon disulfide (0.28 mL, 4.6 mmol) in dry tetrahydrofuran (5 mL) was added drop wise to a stirred solution of potassium tert-butoxide (0.34 g, 3.1 mmol) in dry tetrahydrofuran (30 mL) at −78° C. The mixture was allowed to reach room temperature over 30 minutes. Concentration in vacuo, extraction between ethyl acetate and brine, drying over sodium sulphate and evaporation in vacuo gave 0.99 g (quantitative yield) of the title compound: MS m/z (APCI) 453 [M+1]⁺.

### Example 140

### 4-[4-(3-Bromophenyl)-2,5-dithioxo-1,3-thiazolidin-4-yl]-N,N-dimethylbenzamide

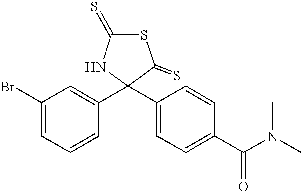

The title compound was synthesized in quantitative yield (0.055 g) as described in example 139 starting from 4-[(3-bromophenyl)(isothiocyanato)methyl]-N,N-dimethylbenzamide: MS m/z (APCI) 453 [M+1]⁺.

### Example 141

### 3-[8-(3-Bromophenyl)-3,3-difluoro-6-thioxo-2,3,4,6,7,8-hexahydroimidazo[1,5-a]pyrimidin-8-yl]-N,N-dimethylbenzamide

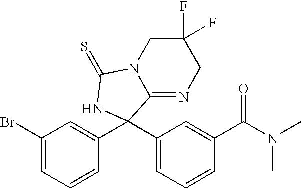

3-[4-(3-Bromophenyl)-2,5-dithioxo-1,3-thiazolidin-4-yl]-N,N-dimethylbenzamide (0.20 g, 0.44 mmol), 2,2′-difluoro-1,3-diaminopropane hydrochloride (0.080 g, 0.44 mmol) and triethylamine (0.18 mL, 1.3 mmol) were mixed in ethanol (5 mL) and heated to 70° C. for 12 h. The mixture was concentrated in vacuo and the residue was diluted with ethyl acetate and washed with first aqueous sodium carbonate, then with brine, dried over sodium sulfate and the solvent was evaporated. Purification by chromatography on silica using ethyl acetate in heptane (0-100%) gave 0.21 g (quantitative yield) of the title compound:

### Example 142

### 4-[8-(3-Bromophenyl)-3,3-difluoro-6-thioxo-2,3,4,6,7,8-hexahydroimidazo[1,5-a]pyrimidin-8-yl]-N,N-dimethylbenzamide

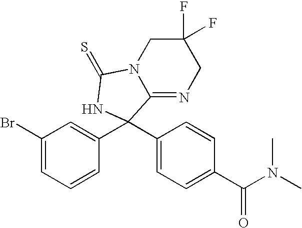

The title compound was synthesized in quantitative yield (0.060 g) as described in example 141 starting from 4-[4-(3-bromophenyl)-2,5-dithioxo-1,3-thiazolidin-4-yl]-NAN-dimethylbenzamide: MS m/z (APCI) 495 [M+1]⁺.

### Example 143

### 3-[6-Amino-8-(3-bromophenyl)-3,3-difluoro-2,3,4,8-tetrahydroimidazo[1,5-a]pyrimidin-8-yl]-N,N-dimethylbenzamide

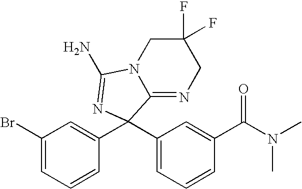

3-[8-(3-Bromophenyl)-3,3-difluoro-6-thioxo-2,3,4,6,7,8-hexahydroimidazo[1,5-a]pyrimidin-8-yl]-N,N-dimethylbenzamide (0.21 g, 0.44 mmol) was dissolved in methanol (8 mL) and ammonium hydroxide (30% in aqueous solution, 4 mL) and tert-butyl hydroperoxide (70% in aqueous solution, 1.2 mL, 8.8 mmol) was added. The reaction was heated at 40° C. for 12 h. Concentration in vacuo, extraction between ethyl acetate and water, drying over sodium sulphate and evaporation of the solvent in vacuo gave 0.21 g (quantitative yield) of the title compound: MS m/z (APCI) 478 [M+1]⁺.

### Example 144

### 4-≡6-Amino-8-(3-bromophenyl)-3,3-difluoro-2,3,4,8-tetrahydroimidazo[1,5-a]pyrimidin-8-yl]-N,N-dimethylbenzamide

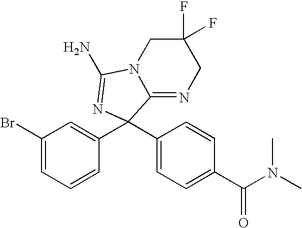

The title compound was synthesized in quantitative yield (0.060 g) as described in example 143 starting from 3-[8-(3-bromophenyl)-3,3-difluoro-6-thioxo-2,3,4,6,7,8-hexahydroimidazo-[1,5-a]pyrimidin-8-yl]-N,N-dimethylbenzamide: MS m/z (APCI) 478 [M+1]⁺.

### Example 145

### 3-{6-Amino-3,3-difluoro-8-[3-(2-fluoropyridin-3-yl)phenyl]-2,3,4,8-tetrahydroimidazo[1,5-a]pyrimidin-8-yl}-N,N-dimethylbenzamide

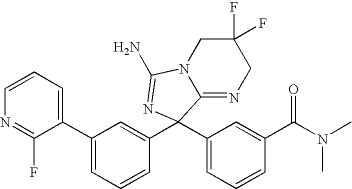

3-[6-Amino-8-(3-bromophenyl)-3,3-difluoro-2,3,4,8-tetrahydroimidazo[1,5-a]pyrimidin-8-yl]-N,N-dimethylbenzamide (0.11 g, 0.24 mmol) was dissolved in 1,2-dimethoxyethane:water:ethanol (6:3:1, 3 mL), and 2-fluoro-3-pyridylboronic acid (0.067 g, 0.48 mmol) and cesium carbonate (0.23 g, 0.71 mmol) was added. Nitrogen was bubbled through the solution for 5 minutes. [1,1′-Bis(diphenylphosphino)ferrocene]palladium(II) chloride (0.020 g, 0.02 mmol) was added and the reaction was heated at 130° C. under nitrogen atmosphere for 20 minutes in a microwave oven. Concentration in vacuo, aqueous workup with ethyl acetate and water and evaporation of the solvent in vacuo followed by purification by preparative HPLC, gave 0.030 g (25%) of the title compound: ¹H NMR (CD₃OD) δ 8.17 (m, 1H), 8.00 (m, 1H), 7.59 (m, 1H), 7.57-7.53 (m, 2H), 7.51-7.43 (m, 4H), 7.42-7.36 (m, 2H), 4.06 (m, 2H), 3.84 (m, 2H), 3.07-2.94 (m, 6H). MS m/z (APCI) 493 [M+1]⁺.

### Example 146

### 4-{6-Amino-3,3-difluoro-8-[3-(2-fluoropyridin-3-yl)phenyl]-2,3,4,8-tetrahydroimidazo[1,5-a]pyrimidin-8-yl}-N,N-dimethylbenzamide

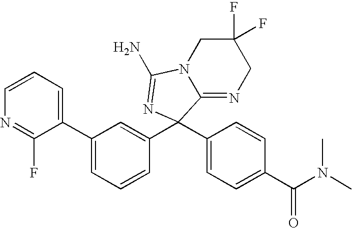

The title compound was synthesized in 11% yield as described in example 145 starting from 2-fluoro-3-pyridylboronic acid: ¹H NMR (CD₃OD) δ 8.17 (m, 1H), 8.00 (m, 1H), 7.59 (m, 1H), 7.56 (m, 1H), 7.54-7.41 (m, 6H), 7.39 (m, 1H), 4.06 (m, 2H), 3.84 (m, 2H), 3.09-2.99 (m, 6H). MS m/z (APCI) 493 [M+1]⁺.

Assays

Compounds were tested in at least one of the following assays:

β-Secretase Enzyme

The enzyme used in the IGEN Cleavage-, Fluorescent-, TR-FRET- and BiaCore assays is described as follows:

The soluble part of the human β-secretase (AA 1-AA 460) was cloned into the ASP2-F10-1-IRES-GFP-neoK mammalian expression vector. The gene was fused to the Fc domain of IgG1 (affinity tag) and stably cloned into HEK 293 cells. Purified sBACE-Fc is stored in Tris buffer, pH 9.2 and has a purity of 95%.

IGEN Cleavage Assay

The enzyme was diluted to 43 μg/ml in 40 mM MES pH 5.0. The IGEN substrate was diluted to 12 μM in 40 mM MES pH 5.0. Compounds were diluted to the desired concentration in dimethyl sulfoxide (final dimethyl sulfoxide concentration in assay is 5%). The assay was performed in a 96 well PCR plate from Greiner (#650201). Compound in dimethyl sulfoxide (3 μL) and enzyme (27 μL) were added to the plate, and pre-incubated for 10 min. The reaction was started with substrate (30 μL). The final dilution of enzyme was 20 μg/ml and the final concentration of substrate was 6 μM. After 20 minutes reaction at room temperature (RT), the reaction was stopped by removing 10 μL of the reaction mix and diluting it 1:25 in 0.2 M Trizma-HCl, pH 8.0. The product was quantified by adding 50 μL of a 1:5000 dilution of the neoepitope antibody to 50 μL of the 1:25 dilution of the reaction mix (all antibodies and the streptavidin coated beads were diluted in PBS containing 0.5% BSA and 0.5% Tween20). Then, 100 μL of 0.2 mg/mL streptavidin coated beads (Dynabeads M-280) and a 1:5000 dilution of ruthenylated goat anti-rabbit (Ru-GαR) antibody was added. The mixture was measured for electro-chemiluminescence in a BioVeris M8 Analyzer after 2 hours of incubation with shaking at RT. The dimethyl sulfoxide control defined 100% activity level and 0% activity was defined by exclusion of the enzyme (using 40 mM MES pH 5.0 buffer instead).

Fluorescent Assay

The enzyme was diluted to 52 μg/ml in 40 mM MES pH 5.0. The substrate (Dabcyl-Edans) was diluted to 30 μM in 40 mM MES pH 5.0. Compounds were diluted to the desired concentration in dimethyl sulfoxide (final dimethyl sulfoxide concentration in assay is 5%). The assay is done in a Corning 384 well round bottom, low volume, non-binding surface plate (Corning #3676). Enzyme (9 μL) together with 1 μL of compound in dimethyl sulfoxide were added to the plate and pre-incubated for 10 min. Substrate (10 μL) was added and the reaction proceeded in the dark at RT for 25 min. The final dilution of enzyme was 23 μg/ml, and the final concentration of substrate was 15 μM (Km of 25 μM). The fluorescence of the product was measured on a Victor II plate reader with an excitation wavelength of 360 nm and an emission wavelength of 485 nm using a protocol for labelled Edans peptide. The dimethyl sulfoxide control defined 100% activity level and 0% activity was defined by exclusion of the enzyme (using 40 mM MES pH 5.0 buffer instead).

TR-FRET Assay

Enzyme was diluted to 6 μg/mL and the substrate (Europium)CEVNLDAEFK(Qsy7) to 200 nM in reaction buffer (NaAcetate, chaps, triton x-100, EDTA pH 4.5). Compounds were diluted to the desired concentration in dimethyl sulfoxide (final dimethyl sulfoxide concentration in assay is 5%). The assay was done in a Costar 384 well round bottom, low volume, non-binding surface plate (Corning #3676). Enzyme (9 μL) and 1 μL of compound in dimethyl sulfoxide was added to the plate, mixed and pre-incubated for 10 min. Substrate (10 μL) was added and the reaction proceeded in the dark for 15 min at RT. The reaction was stopped with the addition of 7 μL NaAcetate, pH 9. The fluorescence of the product was measured on a Victor II plate reader with an excitation wavelength of 340 nm and an emission wavelength of 615 nm. The final concentration of the enzyme was 2.7 μg/ml and the final concentration of the substrate was 100 nM (Km of 290 nM). The dimethyl sulfoxide control defined the 100% activity level and 0% activity was defined by exclusion of the enzyme (using reaction buffer instead).

BACE Biacore Sensor Chip Preparation

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was coupled to channel 2 of the same chip. The two peptides were dissolved at 0.2 mg/mL in 20 mM sodium acetate pH 4.5, and then the solutions were centrifuged at 14K rpm to remove any particulates. Carboxyl groups on the dextran layer were activated by injecting a one to one mixture of 0.5 M N-ethyl-N′ (3-dimethylaminopropyl)-carbodiimide and 0.5 M N-hydroxysuccinimide at 5 μL/min for 7 min. Then the stock solution of the control peptide was injected in channel 1 for 7 min at 5 μL/min., and then the remaining activated carboxyl groups were blocked by injecting 1 M ethanolamine for 7 min at 5 μL/min.

BACE Biacore Assay Protocol

The BACE Biacore assay was done by diluting BACE to 0.5 μM in sodium acetate buffer at pH 4.5 (running buffer minus dimethyl sulfoxide). The diluted BACE was mixed with dimethyl sulfoxide or compound diluted in dimethyl sulfoxide at a final concentration of 5% dimethyl sulfoxide. The BACE/inhibitor mixture was incubated for 30 minutes at RT before being injected over channel 1 and 2 of the CM5 Biacore chip at a rate of 20 μL/min.

As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The dimethyl sulfoxide control was defined as 100% and the effect of the compound was reported as percent inhibition of the dimethyl sulfoxide control.

Beta-Secretase Whole Cell Assays

Generation of HEK293-APP695

The pcDNA3.1 plasmid encoding the cDNA of human full-length APP695 was stably transfected into HEK-293 cells using the Lipofectamine transfection reagent according to manufacture's protocol (Invitrogen). Colonies were selected with 0.1-0.5 mg/mL of zeocin. Limited dilution cloning was performed to generate homogeneous cell lines. Clones were characterized by levels of APP expression and Aβ secreted in the conditioned media using an ELISA assay developed in-house.

Cell Culture for HEK293-APP695

HEK293 cells stably expressing human wild-type APP (HEK293-APP695) were grown at 37° C., 5% CO₂ in DMEM containing 4500 g/L glucose, GlutaMAX and sodium pyruvate supplemented with 10% FBS, 1% non-essential amino acids and 0.1 mg/mL of the selection antibiotic zeocin.

Aβ40 Release Assay

HEK293-APP695 cells were harvested at 80-90% confluence and seeded at a concentration of 0.2×10⁶ cells/mL, 100 mL cell suspension/well, onto a black clear bottom 96-well poly-D-lysine coated plate. After over night incubation at 37° C., 5% CO₂, the cell medium was replaced with cell culture medium with penicillin and streptomycin (100 U/mL, 100 μg/mL, respectively) containing test compounds in a final dimethyl sulfoxide concentration of 1%. Cells were exposed to the test compounds for 24 h at 37° C., 5% CO₂. To quantify the amount of released Aβ, 100 μL cell medium was transferred to a round bottom polypropylene 96-well plate (assay plate). The cell plate was saved for the ATP assay, as described below. To the assay plate, 50 μL of primary detection solution containing 0.5 μg/mL of the rabbit anti-Aβ40 antibody and 0.5 μg/mL of the biotinylated monoclonal mouse 6E10 antibody in DPBS with 0.5% BSA and 0.5% Tween-20 was added per well and incubated over night at 4° C. Then, 50 μL of secondary detection solution containing 0.5 μg/mL of a ruthenylated goat anti-rabbit antibody and 0.2 mg/mL of streptavidin coated beads (Dynabeads M-280) was added per well. The plate was vigorously shaken at RT for 1-2 hours. The plate was then measured for electro-chemiluminescence in a BioVeris M8 Analyzer.

Cell Culture for SH-SY5Y

SH-SY5Y cells were grown 37° C. with 5% CO₂ in DMEM/F-12 1:1 containing GlutaMAX supplemented with 1 mM HEPES, 10% FBS and 1% non-essential amino acids.

sAPPβ Release Assay

SH-SY5Y cells were harvested at 80-90% confluence and seeded at a concentration of 1.5×10⁶ cells/mL, 100 mL cell suspension/well, onto a black clear flat bottom 96-well tissue culture plate. After 7 hours of incubation at 37° C., 5% CO₂, the cell medium was replaced with 90 μl cell culture medium with penicillin and streptomycin (100 U/mL, 100 μg/mL, respectively) containing test compounds in a final dimethyl sulfoxide concentration of 1%. Cells were exposed to the test compounds for 18 h at 37° C., 5% CO₂. To measure sAPPβ released into the cell medium, sAPPβ microplates from Meso Scale Discovery (MSD) were used and the assay was performed according to the manufacture's protocol. Briefly, 25 μL cell medium was transferred to a previously blocked MSD sAPPβ microplate. The cell plate was saved for the ATP assay, as described below. The sAPPβ was captured during shaking at RT for 1 hour, by antibodies spotted in the wells of the microplate. After multiple washes, SULFO-TAG labeled detection antibody was added (25 μL/well, final concentration 1 nM) to the assay plate and the plate was incubated with shaking at RT for 1 hour. Following multiple washes, 150 μl/well of Read Buffer T was added to the plate. After 10 minutes at RT the plate was read in the SECTOR™ Imager for electro-chemiluminescence.

ATP Assay

As indicated above, after transferring medium for analysis of Aβ40 or sAPPβ from the cell plate, the plate was used to analyze cytotoxicity using the ViaLight™ Plus cell proliferation/cytotoxicity kit from Cambrex BioScience that measures total cellular ATP. The assay was performed according to the manufacture's protocol. Briefly, 50 μL cell lysis reagent was added per well. The plates were incubated at RT for 10 min. Two min after addition of 100 μL reconstituted ViaLight™ Plus ATP reagent, the luminescence was measured in a Wallac Victor² 1420 multilabel counter.

hERG Assay

Cell Culture

The hERG-expressing Chinese hamster ovary K1 (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-hERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 μg/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 μl of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound hERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound hERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset was adjusted by determining the hERG tail current reversal potential in IonWorks™ HT, comparing it with that found in conventional electrophysiology (−82 mV) and then making the necessary offset adjustment in the IonWorks™ HT software. The current signal was sampled at 2.5 kHz. Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan hERG tail current amplitude >150 pA; post-scan seal resistance >60 MΩ. The degree of inhibition of the hERG current was assessed by dividing the post-scan hERG current by the respective pre-scan hERG current for each well.

Results

Typical IC50 values for the compounds of the present invention are in the range of about 1 to about 10,000 nM. Biological data on exemplified final compounds is given below in Table 1.

TABLE 1

| | IC50 in |
|---|---|
| Example | TR-FRET assay |
| No. | (nM) |
| 31 | 156 |
| 32 | 501 |
| 33 | 397 |
| 34 | 161 |
| 35 | 463 |
| 36 | 92 |
| 37 | 1116 |
| 38 | 1155 |
| 39 | 1163 |
| 40 | 436 |
| 41 | 981 |
| 42 | 284 |
| 43 | 142 |
| 44 | 471 |
| 45 | 529 |
| 46 | 826 |
| 47 | 658 |
| 48 | 757 |
| 49 | 819 |
| 50 | 45 |
| 51 | 143 |
| 52 | 43 |
| 53 | 55 |
| 54 | 347 |
| 55 | 139 |
| 56 | 38 |
| 57 | 71 |
| 58 | 339 |
| 59 | 221 |
| 60 | 493 |
| 61 | 671 |
| 62 | 254 |
| 63 | 912 |
| 64 | 96 |
| 65 | 248 |
| 66 | 66 |
| 67 | 61 |
| 68 | 422 |
| 69 | 313 |
| 70 | 620 |
| 71 | 390 |
| 72 | 644 |
| 73 | 190 |
| 74 | 449 |
| 75 | 146 |
| 76 | 226 |
| 77 | 1530 |
| 78 | 844 |
| 79 | 436 |
| 80 | 1375 |
| 81 | 417 |
| 82 | 484 |
| 83 | 2512 |
| 84 | 2507 |
| 85 | 1031 |
| 86 | 107 |
| 87 | 57 |
| 90 | 113 |
| 98 | 727 |
| 113 | 1204 |
| 114 | 246 |
| 115 | 106 |
| 116 | 285 |
| 117 | 252 |
| 118 | 645 |
| 119 | 754 |
| 120 | 272 |
| 121 | 893 |
| 122 | 69 |
| 123 | 106 |
| 124 | 528 |
| 125 | 51 |
| 126 | 156 |
| 127 | 177 |
| 128 | 214 |
| 129 | 464 |
| 130 | 137 |
| 131 | 538 |
| 132 | 375 |
| 133 | 375 |
| 145 | 224 |
| 146 | 214 |

1. A compound of formula J: 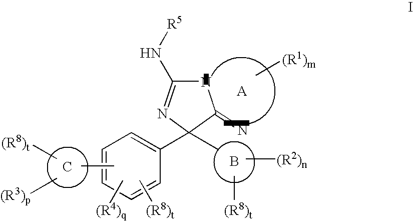
wherein
A is independently selected from a 5, 6 or 7 membered heterocyclic ring optionally substituted with one or more R¹;
B is independently selected from phenyl or from a 5 or 6 membered heteroaromatic ring optionally substituted with one or more R²;
C is independently selected from phenyl or a 5 or 6 membered heteroaromatic ring optionally substituted with one or more R³;
R¹ is independently selected from halogen, cyano, nitro, OR⁶, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl, NR⁶R₇, CONR⁶R⁷, NR⁶(CO)R⁷, (CO)R⁶, CO₂R⁶, COR⁶, (SO₂)NR⁶R⁷, NR⁶(SO₂)R⁷, SOR⁶, SO₂R⁶, OSO₂R⁶ and SO₃R⁶ wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl and C₀₋₆alkylC₃₋₆heterocyclyl is optionally substituted with one or more E; or two R¹ substituents together with the atom to which they are attached, form a cyclic or heterocyclic ring optionally substituted with one or more E;
R², R³ or R⁴ is selected from aryl, heteroaryl, C₃₋₆cycloalkenyl, C₃₋₆cycloalkynyl, C₃₋₆heterocyclyl, CONR⁶R⁷, NR⁶(CO)R⁷, O(CO)R⁶, CO₂R⁶, COR⁶, (SO₂)NR⁶R⁷, NR⁶(SO₂)R⁷, SOR⁶, SO₂R⁶, OSO₂R⁶ and SO₃R⁶ wherein said aryl, heteroaryl, C₃₋₆cycloalkenyl, C₃₋₆cycloalkynyl and C₃₋₆heterocyclyl may be optionally substituted with one or more E;
R⁵ is independently selected from hydrogen, cyano, OR⁶, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl, CONR⁶R⁷, CO₂R⁶, COR⁶, SO₂R⁶ and SO₃R⁶ wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl may be optionally substituted with one or more E;
E is independently selected from halogen, nitro, CN, OR⁶, C₁₋₆alkyl, C₂₋₆alkenyl, is C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl, fluoromethyl, difluoromethyl, trifluoromethyl, fluoromethoxy, difluoromethoxy, trifluoromethoxy, NR⁶R⁷, CONR⁶R⁷, NR⁶(CO)R⁷, O(CO)R⁶, CO₂R⁶, COR⁶, (SO₂)NR⁶R⁷, NR⁶SO₂R⁷, SO₂R⁶, SOR⁶, OSO₂R⁶ and SO₃R⁶, wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl or C₀₋₆alkylheterocyclyl may be optionally substituted with one or more substituents independently selected from halo, nitro, cyano, OR⁶, C₁₋₆alkyl, fluoromethyl, difluoromethyl, trifluoromethyl, fluoromethoxy, difluoromethoxy and trifluoromethoxy;
R⁶ and R⁷ are independently selected from hydrogen, C₁₋₆alkyl, fluoromethyl, difluoromethyl, trifluoromethyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl, or
R⁶ and R⁷ may together form a 5 or 6 membered heterocyclic ring containing one or more heteroatoms selected from N, O or S;
R⁸ is independently selected from halogen, cyano, nitro, OR⁹, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylC₃₋₆heterocyclyl, NR⁹R¹⁰, CONR⁹R¹⁰, NR⁹(CO)R¹⁰, O(CO)R⁹, CO₂R⁹, COR⁹, (SO₂)NR⁹R¹⁰, NR⁹(SO₂)R¹⁰, SO₂R⁹, SOR⁹, OSO₂R⁹ and SO₃R⁹ wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl and C₀₋₆alkylC₃₋₆heterocyclyl may be optionally substituted with one or more F; or
two R⁸ may together with the atoms to which they are attached form a cyclic or heterocyclic ring optionally substituted with one or more F;
R⁹ and R¹⁰ are independently selected from hydrogen, C₁₋₆alkyl, fluoromethyl, difluoromethyl, trifluoromethyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl; or
R⁹ and R¹⁰ may together form a 5 or 6 membered heterocyclic ring containing one or more heteroatoms selected from N, O or S;
m=0, 1 or 2;
n=0, 1, 2 or 3;
p=0, 1, 2 or 3;
q=0, 1, 2 or 3;
t=0, 1, 2 or 3;
wherein one of n, p or q is at least 1;
as a free base or a pharmaceutically acceptable salt, solvate or solvate of a salt thereof.

2. A compound according to claim 1, wherein
A is independently selected from a 5 or 6 membered heterocyclic ring;
B is independently selected from phenyl or from a 5 or 6 membered hetero aromatic ring optionally substituted with one or more R²;
C is independently selected from phenyl or a 5 or 6 membered heteroaromatic ring optionally substituted with one or more R³;
R², R³ or R⁴ is independently selected from aryl, heteroaryl, C₃₋₆cycloalkenyl, C₃₋₆cycloalkynyl, C₃₋₆heterocyclyl, CONR⁶R⁷, NR⁶(CO)R⁷, O(CO)R⁶, CO₂R⁶, COR⁶, (SO₂)NR⁶R⁷, NR⁶(SO₂)R⁷, SOR⁶, SO₂R⁶, OSO₂R⁶ and SO₃R⁶ wherein said aryl, heteroaryl, C₃₋₆cycloalkenyl, C₃₋₆cycloalkynyl and C₃₋₆heterocyclyl may be optionally substituted with one or more E;
R⁵ is hydrogen;
E is independently selected from halogen, nitro, CN, OR⁶, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl, fluoromethyl, difluoromethyl, trifluoromethyl, fluoromethoxy, difluoromethoxy, trifluoromethoxy, NR⁶R₇, CONR⁶R⁷, NR⁶(CO)R⁷, O(CO)R⁶, CO₂R⁶, COR⁶, (SO₂)NR⁶R⁷, NR⁶SO₂R⁷, SO₂R⁶, SOR⁶, OSO₂R⁶ and SO₃R⁶, wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl or C₀₋₆alkylheterocyclyl may be optionally substituted with one or more substituents independently selected from halo, nitro, cyano, OR⁶, C₁₋₆alkyl, fluoromethyl, difluoromethyl, trifluoromethyl, fluoromethoxy, difluoromethoxy and trifluoromethoxy;
R⁶ and R⁷ are independently selected from hydrogen, C₁₋₆alkyl, fluoromethyl, difluoromethyl, trifluoromethyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl, or
R⁶ and R₇ may together form a 5 or 6 membered heterocyclic ring containing one or more heteroatoms selected from N, O or S;
R⁸ is independently selected from halogen, cyano, nitro, OR⁹, C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆-cycloalkenyl, C₀₋₆alkylC₃₋₆cycloalkynyl, C₀₋₆alkylC₃₋₆heterocyclyl, NR⁹R¹⁰, CONR⁹R¹⁰, NR⁹(CO)R¹⁰, O(CO)R⁹, CO₂R⁹, COR⁹, (SO₂)NR⁹R¹⁰, NR⁹(SO₂)R¹⁰, SO₂R⁹, SOR⁹, OSO₂R⁹ and SO₃R⁹ wherein said C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl and C₀₋₆alkylC₃₋₆heterocyclyl may be optionally substituted with one or more E; or
two R⁸ may together with the atoms to which they are attached form a cyclic or heterocyclic ring optionally substituted with one or more E;
R⁹ and R¹⁰ are independently selected from hydrogen, C₁₋₆alkyl, fluoromethyl, difluoromethyl, trifluoromethyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylC₃₋₆cycloalkenyl, C₀₋₆alkylC₃₋₆cycloalkynyl, C₀₋₆alkylheterocyclyl; or
R⁹ and R¹⁰ may together form a 5 or 6 membered heterocyclic ring containing one or more heteroatoms selected from N, O or S;
m=0
n=0 or 1;
p=0 or 1;
q=0, 1, 2 or 3;
t=0, 1, 2 or 3;
wherein one of n, p or q is at least 1.

3. A compound according to claim 1 or 2, wherein B is independently selected from phenyl or a 6 membered heteroaromatic ring optionally substituted with one R².

4. A compound according to claim 1 or 2, wherein B is independently selected from phenyl and pyridyl optionally substituted with one R².

5. A compound according to claim 1 or 2, wherein C is independently selected from phenyl or a 6 membered heteroaromatic ring optionally substituted with one R³.

6. A compound according to claim 1, wherein n is 1 and R² is OSO₂R⁶.

7. A compound according to claim 1, wherein B is independently selected from phenyl and pyridyl; n is 1 and R² is OSO₂R⁶.

8. A compound according to claim 1, wherein R³ is OSO₂R⁶.

9. A compound according to claim 1, wherein C is independently selected from phenyl or a 6 membered heteroaromatic ring; p is 1 and R³ is OSO₂R⁶.

10. A compound according to claim 1, wherein R⁶ is C₁₋₆alkyl.

11. A compound according to claim 1, wherein R⁶ is trifluoromethyl.

12. A compound according to claim 1, wherein m is 0; n is 0; p is 0; and q is 1.

13. A compound according to claim 1, wherein m is 0; n is 1; p is 0; and q is 0.

14. A compound according to claim 1, wherein m is 0; n is 0; p is 1; and q is 0.

15. A compound according to claim 1 or 2, wherein t is 1 or 2.

16. A compound according to claim 1, wherein R⁸ is located on C, and is independently selected from halogen, cyano, nitro and OR⁹.

17. A compound according to claim 1, wherein R⁹ is selected from C₁₋₆alkyl and trifluoromethyl.

18. A compound according to claim 1, wherein R⁸ is located on C, and is C₁₋₆alkyl optionally substituted with one or more E.

19. A compound according to claim 1, wherein E is halogen.

20. A compound according to claim 1, wherein
A is selected from a 5 or 6 membered heterocyclic ring;
B is selected from phenyl or a 6 membered heteroaromatic ring optionally substituted with one R²;
C is selected from phenyl or a 6 membered heteroaromatic ring optionally substituted with one or more R³;
R² or R³ is OSO₂R⁶;
R⁸ is hydrogen;
R⁶ is C₁₋₆alkyl;
R⁸ is selected from halogen, cyano, nitro, OR⁹;
R⁹ is selected from C₁₋₆alkyl and trifluoromethyl;
m=0;
n=0 or 1;
p=0;
q=0, 1 or 2;
t=0 or 1;
wherein one of n or q is at least 1.

21. A compound according to claim 1, wherein
A is independently selected from a 5 or 6 membered heterocyclic ring;
B is phenyl, optionally substituted with one R²;
C is independently selected from phenyl or a 6 membered heteroaromatic ring optionally substituted with one or more R³;
R² or R³ is OSO₂R⁶;
R⁵ is hydrogen;
E is halogen;
R⁶ is independently selected from C₁₋₆alkyl and trifluoromethyl;
R³ is independently selected from halogen, OR⁹ and C₁₋₆alkyl, wherein said C₁₋₆alkyl is optionally substituted with one or more E;
R⁹ is independently selected from hydrogen and C₁₋₆alkyl;
m=0;
n=0 or 1;
p=0 or 1;
q=0;
t=0, 1 or 2;
wherein one of n or p is at least 1.

22. A compound according to claim 1, wherein
A is a 6 membered heterocyclic ring substituted with two R¹;
B is phenyl substituted with one R²;
C is a 6 membered heteroaromatic ring;
R¹ is halogen;
R² is CONR⁶R⁷;
R⁵ is hydrogen;
R⁶ and R⁷ are C₁₋₆alkyl;
is R⁸ is halogen;
m=2;
n=1;
p=0;
q=0; and
t=0 or 1.

23. A compound, selected from:
4-[6-Amino-8-(3′-methoxybiphenyl-3-yl)-2,3,4,8-tetrahydroimidazo[1,5-a]pyrimidin-8-yl]phenyl methanesulfonate acetate;
4-[6-Amino-8-(3-pyrazin-2-ylphenyl)-2,3,4,8-tetrahydroimidazo[1,5-a]pyrimidin-8-yl]phenyl methanesulfonate acetate;
4-{6-Amino-8-[3-(5-fluoropyridin-3-yl)phenyl]-2,3,4,8-tetrahydroimidazo[1,5-a]pyrimidin-8-yl}phenyl methanesulfonate acetate;
4-{6-Amino-8-[3-(5-methoxypyridin-3-yl)phenyl]-2,3,4,8-tetrahydroimidazo[1,5-a]pyrimidin-8-yl}phenyl methanesulfonate acetate;
4-[6-Amino-8-(3′-cyanobiphenyl-3-yl)-2,3,4,8-tetrahydroimidazo[1,5-a]pyrimidin-8-yl]phenyl methanesulfonate acetate;
4-[6-Amino-8-(3′-chlorobiphenyl-3-yl)-2,3,4,8-tetrahydroimidazo[1,5-a]pyrimidin-8-yl]phenyl methanesulfonate 0.25 acetate;
4-{6-Amino-8-[3-(6-fluoropyridin-3-yl)phenyl]-2,3,4,8-tetrahydroimidazo[1,5-a]pyrimidin-8-yl}phenyl methanesulfonate 0.25 acteate;
4-{6-Amino-8-[3-(2,6-difluoropyridin-3-yl)phenyl]-2,3,4,8-tetrahydroimidazo[1,5-a]pyrimidin-8-yl}phenyl methanesulfonate 0.25 acetate;
4-[6-Amino-8-(3-pyridin-3-ylphenyl)-2,3,4,8-tetrahydroimidazo[1,5-a]pyrimidin-8-yl]phenyl methanesulfonate 0.75 acetate;
4-{6-Amino-8-[3-(2-fluoropyridin-3-yl)phenyl]-2,3,4,8-tetrahydroimidazo[1,5-a]pyrimidin-8-yl}phenyl methanesulfonate 0.25 acetate;
4-{6-Amino-8-[3′-(trifluoromethoxy)biphenyl-3-yl]-2,3,4,8-tetrahydroimidazo[1,5-a]pyrimidin-8-yl}phenyl methanesulfonate 0.5 acetate;
4-[6-Amino-8-(2′-fluoro-3′-methoxybiphenyl-3-yl)-2,3,4,8-tetrahydroimidazo[1,5-a]pyrimidin-8-yl]phenyl methanesulfonate 0.5 acetate;
4-[6-Amino-8-(2′-fluoro-5′-methoxybiphenyl-3-yl)-2,3,4,8-tetrahydroimidazo[1,5-a]pyrimidin-8-yl]phenyl methanesulfonate 0.25 acetate;
4-[6-Amino-8-(3′-ethoxybiphenyl-3-yl)-2,3,4,8-tetrahydroimidazo[1,5-a]pyrimidin-8-yl]phenyl methanesulfonate 0.5 acetate;
4-[6-Amino-8-(3′-nitrobiphenyl-3-yl)-2,3,4,8-tetrahydroimidazo[1,5-a]pyrimidin-8-yl]phenyl methanesulfonate 0.5 acetate;
4-[6-Amino-8-(2′,5′-dimethoxybiphenyl-3-yl)-2,3,4,8-tetrahydroimidazo[1,5-a]pyrimidin-8-yl]phenyl methanesulfonate 0.5 acetate;
4-[6-Amino-8-(3′-cyano-4′-fluorobiphenyl-3-yl)-2,3,4,8-tetrahydroimidazo[1,5-a]pyrimidin-8-yl]phenyl methanesulfonate 0.5 acetate;
4-[6-Amino-8-(5′-cyano-2′-fluorobiphenyl-3-yl)-2,3,4,8-tetrahydroimidazo[1,5-a]pyrimidin-8-yl]phenyl methanesulfonate 0.75 acetate;
4-[6-Amino-8-(3-pyrimidin-5-ylphenyl)-2,3,4,8-tetrahydroimidazo[1,5-a]pyrimidin-8-yl]phenyl methanesulfonate 0.5 acetate;
4-[6-Amino-8-(3′,5′-dichlorobiphenyl-3-yl)-2,3,4,8-tetrahydroimidazo[1,5-a]pyrimidin-8-yl]phenyl methanesulfonate acetate;
3-{6-Amino-8-[3-(5-chloro-2-fluoropyridin-3-yl)phenyl]-2,3,4,8-tetrahydroimidazo[1,5-a]pyrimidin-8-yl}phenyl methanesulfonate
3′-[6-Amino-8-(4-methoxyphenyl)-2,3,4,8-tetrahydroimidazo[1,5-a]pyrimidin-8-yl]-5-methoxybiphenyl-3-yl methanesulfonate acetate;
3′-[6-Amino-8-(4-methoxyphenyl)-2,3,4,8-tetrahydroimidazo[1,5-a]pyrimidin-8-yl]-5-chlorobiphenyl-3-yl methanesulfonate acetate;
4-[6-Amino-8-(3-pyrazin-2-ylphenyl)-2,3,4,8-tetrahydroimidazo[1,5-a]pyrimidin-8-yl]phenyl propane-1-sulfonate acetate;
4-[6-Amino-8-(3′-methoxybiphenyl-3-yl)-2,3,4,8-tetrahydroimidazo[1,5-a]pyrimidin-8-yl]phenyl propane-1-sulfonate 0.75 acetate;
4-[6-Amino-8-(3′,5′-dichlorobiphenyl-3-yl)-2,3,4,8-tetrahydroimidazo[1,5-a]pyrimidin-8-yl]phenyl propane-1-sulfonate 0.5 acetate;
4-[6-Amino-8-(3′-chlorobiphenyl-3-yl)-2,3,4,8-tetrahydroimidazo[1,5-a]pyrimidin-8-yl]phenyl propane-1-sulfonate 0.75 acetate;
4-[6-Amino-8-(3-pyridin-3-ylphenyl)-2,3,4,8-tetrahydroimidazo[1,5-a]pyrimidin-8-yl]phenyl propane-1-sulfonate 0.5 acetate;
4-{6-Amino-8-[3-(2-fluoropyridin-3-yl)phenyl]-2,3,4,8-tetrahydroimidazo[1,5-a]pyrimidin-8-yl}phenyl propane-1-sulfonate acetate;
4-{6-Amino-8-[3′-(trifluoromethyl)biphenyl-3-yl]-2,3,4,8-tetrahydroimidazo[1,5-a]pyrimidin-8-yl}phenyl propane-1-sulfonate 0.5 acetate;
4-[6-Amino-8-(4′-fluoro-3′-methoxybiphenyl-3-yl)-2,3,4,8-tetrahydroimidazo[1,5-a]pyrimidin-8-yl]phenyl propane-1-sulfonate 0.75 acetate;
4-[6-Amino-8-(3′-chloro-2′-fluorobiphenyl-3-yl)-2,3,4,8-tetrahydroimidazo[1,5-a]pyrimidin-8-yl]phenyl propane-1-sulfonate 0.75 acetate;
4-[6-Amino-8-(2′,5′-dichlorobiphenyl-3-yl)-2,3,4,8-tetrahydroimidazo[1,5-a]pyrimidin-8-yl]phenyl propane-1-sulfonate 0.75 acetate;
4-{6-Amino-8-[3-(5-methoxypyridin-3-yl)phenyl]-2,3,4,8-tetrahydroimidazo[1,5-a]pyrimidin-8-yl}phenyl propane-1-sulfonate 0.75 acetate;
4-[6-Amino-8-(3′-methoxybiphenyl-3-yl)-2,3,4,8-tetrahydroimidazo[1,5-a]pyrimidin-8-yl]phenyl cyclopropane sulfonate 0.75 acetate;
4-[6-Amino-8-(3′,5′-dichlorobiphenyl-3-yl)-2,3,4,8-tetrahydroimidazo[1,5-a]pyrimidin-8-yl]phenyl cyclopropanesulfonate 0.75 acetate;
4-[6-Amino-8-(3′-chlorobiphenyl-3-yl)-2,3,4,8-tetrahydroimidazo[1,5-a]pyrimidin-8-yl]phenyl cyclopropane sulfonate 0.75 acetate;
4-[6-Amino-8-(3-pyridin-3-ylphenyl)-2,3,4,8-tetrahydroimidazo[1,5-a]pyrimidin-8-yl]phenyl cyclopropane sulfonate 0.75 acetate;
4-{6-Amino-8-[3-(2-fluoropyridin-3-yl)phenyl]-2,3,4,8-tetrahydroimidazo[1,5-a]pyrimidin-8-yl}phenyl cyclopropane sulfonate 0.75 acetate;
4-{6-Amino-8-[3′-(trifluoromethyl)biphenyl-3-yl]-2,3,4,8-tetrahydroimidazo[1,5-a]pyrimidin-8-yl}phenyl cyclopropane sulfonate 0.75 acetate;
4-[6-Amino-8-(3′-chloro-2′-fluorobiphenyl-3-yl)-2,3,4,8-tetrahydroimidazo[1,5-a]pyrimidin-8-yl]phenyl cyclopropane sulfonate 0.75 acetate;
4-[6-Amino-8-(2′,5′-dichlorobiphenyl-3-yl)-2,3,4,8-tetrahydroimidazo[1,5-a]pyrimidin-8-yl]phenyl cyclopropane sulfonate 0.5 acetate;
3′-[5-Amino-7-(4-methoxyphenyl)-2,7-dihydro-3H-imidazo[1,5-a]imidazol-7-yl]-5-methoxybiphenyl-3-yl methanesulfonate acetate;
4-[5-Amino-7-(3′-methoxybiphenyl-3-yl)-2,7-dihydro-3H-imidazo[1,5-a]imidazol-7-yl]phenyl methanesulfonate 0.25 acetate;
4-[5-Amino-7-(3′,5′-dichlorobiphenyl-3-yl)-2,7-dihydro-3H-imidazo[1,5-a]imidazol-7-yl]phenyl methanesulfonate 0.25 acetate;
4-[5-Amino-7-(3′-chlorobiphenyl-3-yl)-2,7-dihydro-3H-imidazo[1,5-a]imidazol-7-yl]phenyl methanesulfonate 0.5 acetate;
4-[5-Amino-7-(3-pyridin-3-ylphenyl)-2,7-dihydro-3H-imidazo[1,5-a]imidazol-7-yl]phenyl methanesulfonate 0.5 acetate;
4-{5-Amino-7-[3-(2-fluoropyridin-3-yl)phenyl]-2,7-dihydro-3H-imidazo[1,5-a]imidazol-7-yl}phenyl methanesulfonate 0.5 acetate;
4-{5-Amino-7-[3-(5-chloro-2-fluoropyridin-3-yl)phenyl]-2,7-dihydro-3H-imidazo[1,5-a]imidazol-7-yl}phenyl methanesulfonate 0.5 acetate;
4-[5-Amino-7-(3′-methoxybiphenyl-3-yl)-2,7-dihydro-3H-imidazo[1,5-a]imidazol-7-yl]phenyl propane-2-sulfonate 0.5 acetate;
4-[5-Amino-7-(3′,5′-dichlorobiphenyl-3-yl)-2,7-dihydro-3H-imidazo[1,5-a]imidazol-7-yl]phenyl propane-2-sulfonate 0.5 acetate;
4-[5-Amino-7-(3′-chlorobiphenyl-3-yl)-2,7-dihydro-3H-imidazo[1,5-a]imidazol-7-yl]phenyl propane-2-sulfonate 0.5 acetate;
4-[5-Amino-7-(3-pyridin-3-ylphenyl)-2,7-dihydro-3H-imidazo[1,5-a]imidazol-7-yl]phenyl propane-2-sulfonate 0.75 acetate;
4-{5-Amino-7-[3-(2-fluoropyridin-3-yl)phenyl]-2,7-dihydro-3H-imidazo[1,5-a]imidazol-7-yl}phenyl propane-2-sulfonate 0.75 acetate;
4-{5-Amino-7-[3-(5-methoxypyridin-3-yl)phenyl]-2,7-dihydro-3H-imidazo[1,5-a]imidazol-7-yl}phenyl propane-2-sulfonate 0.5 acetate;
3′-(6-Amino-8-pyridin-4-yl-2,3,4,8-tetrahydroimidazo[1,5-a]pyrimidin-8-yl)-5-chlorobiphenyl-3-yl methanesulfonate 0.5 acetate; and
3′-(6-Amino-8-pyridin-4-yl-2,3,4,8-tetrahydroimidazo[1,5-a]pyrimidin-8-yl)-5-methoxybiphenyl-3-yl methanesulfonate 0.25 acetate.

24. A compound, selected from:
4-[5-Amino-7-(3′-chlorobiphenyl-3-yl)-2,7-dihydro-3H-imidazo[1,5-a]imidazol-7-yl]phenyl trifluoromethanesulfonate 0.75 acetate;
4-[6-Amino-8-(3′-chlorobiphenyl-3-yl)-2,3,4,8-tetrahydroimidazo[1,5-a]pyrimidin-8-yl]phenyl trifluoromethanesulfonate acetate;
3′-(6-Amino-8-phenyl-2,3,4,8-tetrahydroimidazo[1,5-a]pyrimidin-8-yl)-5-methoxybiphenyl-3-yl methanesulfonate hydrochloride;
3-{6-Amino-8-[3′,5′-bis(trifluoromethyl)biphenyl-3-yl]-2,3,4,8-tetrahydroimidazo[1,5-a]pyrimidin-8-yl}phenyl methanesulfonate;
3-[6-Amino-8-(3′-chlorobiphenyl-3-yl)-2,3,4,8-tetrahydroimidazo[1,5-a]pyrimidin-8-yl]phenyl trifluoromethanesulfonate;
3-[6-Amino-8-(3′,5′-dichlorobiphenyl-3-yl)-2,3,4,8-tetrahydroimidazo[1,5-a]pyrimidin-8-yl]phenyl methanesulfonate;
3-[6-Amino-8-(3′-chlorobiphenyl-3-yl)-2,3,4,8-tetrahydroimidazo[1,5-a]pyrimidin-8-yl]phenyl methanesulfonate;
3-[6-Amino-8-(3′-methoxybiphenyl-3-yl)-2,3,4,8-tetrahydroimidazo[1,5-a]pyrimidin-8-yl]phenyl methanesulfonate;
3-[6-Amino-8-(3-pyridin-3-ylphenyl)-2,3,4,8-tetrahydroimidazo[1,5-a]pyrimidin-8-yl]phenyl methanesulfonate;
3-[6-Amino-8-(3-pyrimidin-5-ylphenyl)-2,3,4,8-tetrahydroimidazo[1,5-a]pyrimidin-8-yl]phenyl methanesulfonate;
3-{6-Amino-8-[3-(5-chloro-2-fluoropyridin-3-yl)phenyl]-2,3,4,8-tetrahydroimidazo[1,5-a]pyrimidin-8-yl}phenyl methanesulfonate;
3-[6-Amino-8-(3′,5′-dimethylbiphenyl-3-yl)-2,3,4,8-tetrahydroimidazo[1,5-a]pyrimidin-8-yl]phenyl methanesulfonate;
3-[6-Amino-8-(3′,5′-dichlorobiphenyl-3-yl)-2,3,4,8-tetrahydroimidazo[1,5-a]pyrimidin-8-yl]phenyl propane-1-sulfonate;
3-[6-Amino-8-(3′-methoxybiphenyl-3-yl)-2,3,4,8-tetrahydroimidazo[1,5-a]pyrimidin-8-yl]phenyl propane-1-sulfonate;
3-[6-Amino-8-(3-pyrimidin-5-ylphenyl)-2,3,4,8-tetrahydroimidazo[1,5-a]pyrimidin-8-yl]phenyl propane-1-sulfonate;
3-[6-Amino-8-(3′,5′-dichlorobiphenyl-3-yl)-2,3,4,8-tetrahydroimidazo[1,5-a]pyrimidin-8-yl]phenyl cyclopropanesulfonate;
3-[6-Amino-8-(3′-methoxybiphenyl-3-yl)-2,3,4,8-tetrahydroimidazo[1,5-a]pyrimidin-8-yl]phenyl cyclopropanesulfonate;
3-[6-Amino-8-(3′,5′-dichlorobiphenyl-3-yl)-2,3,4,8-tetrahydroimidazo[1,5-a]pyrimidin-8-yl]phenyl trifluoromethanesulfonate;
3-[6-Amino-8-(3′-methoxybiphenyl-3-yl)-2,3,4,8-tetrahydroimidazo[1,5-a]pyrimidin-8-yl]phenyl trifluoromethanesulfonate;
3-[6-Amino-8-(3-pyridin-3-ylphenyl)-2,3,4,8-tetrahydroimidazo[1,5-a]pyrimidin-8-yl]phenyl trifluoromethanesulfonate;
3-{6-Amino-8-[3-(5-chloro-2-fluoropyridin-3-yl)phenyl]-2,3,4,8-tetrahydroimidazo[1,5-a]pyrimidin-8-yl}phenyl trifluoromethanesulfonate;
3-[6-Amino-8-(3-pyrimidin-5-ylphenyl)-2,3,4,8-tetrahydroimidazo[1,5-a]pyrimidin-8-yl]phenyl trifluoromethanesulfonate; and
3′-[6-Amino-8-(3-methoxyphenyl)-2,3,4,8-tetrahydroimidazo[1,5-a]pyrimidin-8-yl]-5-methoxybiphenyl-3-yl methanesulfonate.

25. A compound, selected from:
3-{6-Amino-3,3-difluoro-8-[3-(2-fluoropyridin-3-yl)phenyl]-2,3,4,8-tetrahydroimidazo[1,5-a]pyrimidin-8-yl}-N,N-dimethylbenzamide; and
4-{6-Amino-3,3-difluoro-8-[3-(2-fluoropyridin-3-yl)phenyl]-2,3,4,8-tetrahydroimidazo[1,5-a]pyrimidin-8-yl}-N,N-dimethylbenzamide.

26. A pharmaceutical composition comprising as active ingredient a therapeutically effective amount of a compound according to claim 1 in association with pharmaceutically acceptable excipients, carriers or diluents.

27. A compound according to claim 1, or a pharmaceutically acceptable salt thereof, for use as a medicament.

28. A method of inhibiting activity of BACE comprising contacting said BACE with a compound according to claim 1.

29. A method of treating or preventing an Aβ-related pathology in a mammal, comprising administering to said patient a therapeutically effective amount of a compound according to claim 1.

30. The method of claim 29, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

31. The method of claim 29, wherein said mammal is a human.

32. A method of treating or preventing an Aβ-related pathology in a mammal, comprising administering to said patient a therapeutically effective amount of a compound according to claim 1, and at least one cognitive enhancing agent, memory enhancing agent, or choline esterase inhibitor.

33. The method of claim 32, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

34. The method of claim 32, wherein said mammal is a human.
